SEQUENCE LISTING (635 sequences)

SEQ ID NO: 1 - RNA, 19 nt, Artificial Sequence
  Synthetic
gggcgaucuuaacauaaua

SEQ ID NO: 2 - DNA, 19 bp, Artificial Sequence
  Synthetic
gggcgatcttaacataata

SEQ ID NO: 3 - RNA, 19 nt, Artificial Sequence
  Synthetic
cccgcuagaauuguauuau

SEQ ID NO: 4 - RNA, 19 nt, Artificial Sequence
  Synthetic
ggcgaucuuaacauaauaa

SEQ ID NO: 5 - DNA, 19 bp, Artificial Sequence
  Synthetic
ggcgatcttaacataataa

SEQ ID NO: 6 - RNA, 19 nt, Artificial Sequence
  Synthetic
ccgcuagaauuguauuauu

SEQ ID NO: 7 - RNA, 19 nt, Artificial Sequence
  Synthetic
ggccugcuaaaggauucaa

SEQ ID NO: 8 - DNA, 19 bp, Artificial Sequence
  Synthetic
ggcctgctaaaggattcaa

SEQ ID NO: 9 - RNA, 19 nt, Artificial Sequence
  Synthetic
ccggacgauuuccuaaguu

SEQ ID NO: 10 - RNA, 19 nt, Artificial Sequence
  Synthetic
gugcagagaaaguaaauaa

SEQ ID NO: 11 - DNA, 19 bp, Artificial Sequence
  Synthetic
gtgcagagaaagtaaataa

SEQ ID NO: 12 - RNA, 19 nt, Artificial Sequence
  Synthetic
cacgucucuuucauuuauu

SEQ ID NO: 13 - RNA, 19 nt, Artificial Sequence
  Synthetic
cacccugucaugaacauau

SEQ ID NO: 14 - DNA, 19 bp, Artificial Sequence
  Synthetic
caccctgtcatgaacatat

SEQ ID NO: 15 - RNA, 19 nt, Artificial Sequence
  Synthetic
gugggacaguacuuguaua

SEQ ID NO: 16 - RNA, 19 nt, Artificial Sequence
  Synthetic
gagccuuuaaaugauuuca

SEQ ID NO: 17 - DNA, 19 bp, Artificial Sequence
  Synthetic
gagcctttaaatgatttca

SEQ ID NO: 18 - RNA, 19 nt, Artificial Sequence
  Synthetic
cucggaaauuuacuaaagu

SEQ ID NO: 19 - RNA, 19 nt, Artificial Sequence
  Synthetic
ccugucaugaacauauuua

SEQ ID NO: 20 - DNA, 19 bp, Artificial Sequence
  Synthetic
cctgtcatgaacatattta

SEQ ID NO: 21 - RNA, 19 nt, Artificial Sequence
  Synthetic
ggacaguacuuguauaaau

SEQ ID NO: 22 - RNA, 19 nt, Artificial Sequence
  Synthetic
gggagugauuauuguuuca

SEQ ID NO: 23 - DNA, 19 bp, Artificial Sequence
  Synthetic
gggagtgattattgtttca

SEQ ID NO: 24 - RNA, 19 nt, Artificial Sequence
  Synthetic
cccucacuaauaacaaagu

SEQ ID NO: 25 - RNA, 19 nt, Artificial Sequence
  Synthetic
gaagcagaaucaucauuua

SEQ ID NO: 26 - DNA, 19 bp, Artificial Sequence
  Synthetic
gaagcagaatcatcattta

SEQ ID NO: 27 - RNA, 19 nt, Artificial Sequence
  Synthetic
cuucgucuuaguaguaaau

SEQ ID NO: 28 - RNA, 19 nt, Artificial Sequence
  Synthetic
gcaacucugagauuauaaa

SEQ ID NO: 29 - DNA, 19 bp, Artificial Sequence
  Synthetic
gcaactctgagattataaa

SEQ ID NO: 30 - RNA, 19 nt, Artificial Sequence
  Synthetic
cguugagacucuaauauuu

SEQ ID NO: 31 - RNA, 19 nt, Artificial Sequence
  Synthetic
guggugcuauagauguaaa

SEQ ID NO: 32 - DNA, 19 bp, Artificial Sequence
  Synthetic
gtggtgctatagatgtaaa

SEQ ID NO: 33 - RNA, 19 nt, Artificial Sequence
  Synthetic
caccacgauaucuacauuu

SEQ ID NO: 34 - RNA, 19 nt, Artificial Sequence
  Synthetic
gcagugcagagaaaguaaa

SEQ ID NO: 35 - DNA, 19 bp, Artificial Sequence
  Synthetic
gcagtgcagagaaagtaaa

SEQ ID NO: 36 - RNA, 19 nt, Artificial Sequence
  Synthetic
cgucacgucucuuucauuu

SEQ ID NO: 37 - RNA, 19 nt, Artificial Sequence
  Synthetic
cugcuuucaucuaugaaau

SEQ ID NO: 38 - DNA, 19 bp, Artificial Sequence
  Synthetic
ctgctttcatctatgaaat

SEQ ID NO: 39 - RNA, 19 nt, Artificial Sequence
  Synthetic
gacgaaaguagauacuuua

SEQ ID NO: 40 - RNA, 19 nt, Artificial Sequence
  Synthetic
gcugucaugaaggcuuucu

SEQ ID NO: 41 - DNA, 19 bp, Artificial Sequence
  Synthetic
gctgtcatgaaggctttct

SEQ ID NO: 42 - RNA, 19 nt, Artificial Sequence
  Synthetic
cgacaguacuuccgaaaga

SEQ ID NO: 43 - RNA, 19 nt, Artificial Sequence
  Synthetic
ggagugauuauuguuucau

SEQ ID NO: 44 - DNA, 19 bp, Artificial Sequence
  Synthetic
ggagtgattattgtttcat

SEQ ID NO: 45 - RNA, 19 nt, Artificial Sequence
  Synthetic
ccucacuaauaacaaagua

SEQ ID NO: 46 - RNA, 19 nt, Artificial Sequence
  Synthetic
gucuguagaaaugucuaau

SEQ ID NO: 47 - DNA, 19 bp, Artificial Sequence
  Synthetic
gtctgtagaaatgtctaat

SEQ ID NO: 48 - RNA, 19 nt, Artificial Sequence
  Synthetic
cagacaucuuuacagauua

SEQ ID NO: 49 - RNA, 19 nt, Artificial Sequence
  Synthetic
cuccggagcauuuggauaa

SEQ ID NO: 50 - DNA, 19 bp, Artificial Sequence
  Synthetic
ctccggagcatttggataa

SEQ ID NO: 51 - RNA, 19 nt, Artificial Sequence
  Synthetic
gaggccucguaaaccuauu

SEQ ID NO: 52 - RNA, 19 nt, Artificial Sequence
  Synthetic
gggauucagucuguagaaa

SEQ ID NO: 53 - DNA, 19 bp, Artificial Sequence
  Synthetic
gggattcagtctgtagaaa

SEQ ID NO: 54 - RNA, 19 nt, Artificial Sequence
  Synthetic
cccuaagucagacaucuuu

SEQ ID NO: 55 - RNA, 19 nt, Artificial Sequence
  Synthetic
gcugaaaccuggcuuaucu

SEQ ID NO: 56 - DNA, 19 bp, Artificial Sequence
  Synthetic
gctgaaacctggcttatct

SEQ ID NO: 57 - RNA, 19 nt, Artificial Sequence
  Synthetic
cgacuuuggaccgaauaga

SEQ ID NO: 58 - RNA, 19 nt, Artificial Sequence
  Synthetic
caguguuccugaagaaaua

SEQ ID NO: 59 - DNA, 19 bp, Artificial Sequence
  Synthetic
cagtgttcctgaagaaata

SEQ ID NO: 60 - RNA, 19 nt, Artificial Sequence
  Synthetic
gucacaaggacuucuuuau

SEQ ID NO: 61 - RNA, 19 nt, Artificial Sequence
  Synthetic
gccuauuccaucacaauca

SEQ ID NO: 62 - DNA, 19 bp, Artificial Sequence
  Synthetic
gcctattccatcacaatca

SEQ ID NO: 63 - RNA, 19 nt, Artificial Sequence
  Synthetic
cggauaagguaguguuagu

SEQ ID NO: 64 - RNA, 19 nt, Artificial Sequence
  Synthetic
cggagcauuuggauaaugu

SEQ ID NO: 65 - DNA, 19 bp, Artificial Sequence
  Synthetic
cggagcatttggataatgt

SEQ ID NO: 66 - RNA, 19 nt, Artificial Sequence
  Synthetic
gccucguaaaccuauuaca

SEQ ID NO: 67 - RNA, 19 nt, Artificial Sequence
  Synthetic
ggagaagugauuccuguaa

SEQ ID NO: 68 - DNA, 19 bp, Artificial Sequence
  Synthetic
ggagaagtgattcctgtaa

SEQ ID NO: 69 - RNA, 19 nt, Artificial Sequence
  Synthetic
ccucuucacuaaggacauu

SEQ ID NO: 70 - RNA, 19 nt, Artificial Sequence
  Synthetic
cgcagcacauauggacuau

SEQ ID NO: 71 - DNA, 19 bp, Artificial Sequence
  Synthetic
cgcagcacatatggactat

SEQ ID NO: 72 - RNA, 19 nt, Artificial Sequence
  Synthetic
gcgucguguauaccugaua

SEQ ID NO: 73 - RNA, 19 nt, Artificial Sequence
  Synthetic
cuggaucauacuccagaau

SEQ ID NO: 74 - DNA, 19 bp, Artificial Sequence
  Synthetic
ctggatcatactccagaat

SEQ ID NO: 75 - RNA, 19 nt, Artificial Sequence
  Synthetic
gaccuaguaugaggucuua

SEQ ID NO: 76 - RNA, 19 nt, Artificial Sequence
  Synthetic
ccugaagauagaccuugau

SEQ ID NO: 77 - DNA, 19 bp, Artificial Sequence
  Synthetic
cctgaagatagaccttgat

SEQ ID NO: 78 - RNA, 19 nt, Artificial Sequence
  Synthetic
ggacuucuaucuggaacua

SEQ ID NO: 79 - RNA, 19 nt, Artificial Sequence
  Synthetic
gccuuagagaauauacuaa

SEQ ID NO: 80 - DNA, 19 bp, Artificial Sequence
  Synthetic
gccttagagaatatactaa

SEQ ID NO: 81 - RNA, 19 nt, Artificial Sequence
  Synthetic
cggaaucucuuauaugauu

SEQ ID NO: 82 - RNA, 19 nt, Artificial Sequence
  Synthetic
cuguagcucagucauuuaa

SEQ ID NO: 83 - DNA, 19 bp, Artificial Sequence
  Synthetic
ctgtagctcagtcatttaa

SEQ ID NO: 84 - RNA, 19 nt, Artificial Sequence
  Synthetic
gacaucgagucaguaaauu

SEQ ID NO: 85 - RNA, 19 nt, Artificial Sequence
  Synthetic
ccaccacacacauagaugu

SEQ ID NO: 86 - DNA, 19 bp, Artificial Sequence
  Synthetic
ccaccacacacatagatgt

SEQ ID NO: 87 - RNA, 19 nt, Artificial Sequence
  Synthetic
ggugguguguguaucuaca

SEQ ID NO: 88 - RNA, 19 nt, Artificial Sequence
  Synthetic
ccaccaaauuuacacaaca

SEQ ID NO: 89 - DNA, 19 bp, Artificial Sequence
  Synthetic
ccaccaaatttacacaaca

SEQ ID NO: 90 - RNA, 19 nt, Artificial Sequence
  Synthetic
ggugguuuaaauguguugu

SEQ ID NO: 91 - RNA, 19 nt, Artificial Sequence
  Synthetic
gcucaggauacgaucaucu

SEQ ID NO: 92 - DNA, 19 bp, Artificial Sequence
  Synthetic
gctcaggatacgatcatct

SEQ ID NO: 93 - RNA, 19 nt, Artificial Sequence
  Synthetic
cgaguccuaugcuaguaga

SEQ ID NO: 94 - RNA, 19 nt, Artificial Sequence
  Synthetic
caccaaauuuacacaacaa

SEQ ID NO: 95 - DNA, 19 bp, Artificial Sequence
  Synthetic
caccaaatttacacaacaa

SEQ ID NO: 96 - RNA, 19 nt, Artificial Sequence
  Synthetic
gugguuuaaauguguuguu

SEQ ID NO: 97 - RNA, 19 nt, Artificial Sequence
  Synthetic
gcagauguuuaauuggaau

SEQ ID NO: 98 - DNA, 19 bp, Artificial Sequence
  Synthetic
gcagatgtttaattggaat

SEQ ID NO: 99 - RNA, 19 nt, Artificial Sequence
  Synthetic
cgucuacaaauuaaccuua

SEQ ID NO: 100 - RNA, 19 nt, Artificial Sequence
  Synthetic
guccuagaguaaggcacau

SEQ ID NO: 101 - DNA, 19 bp, Artificial Sequence
  Synthetic
gtcctagagtaaggcacat

SEQ ID NO: 102 - RNA, 19 nt, Artificial Sequence
  Synthetic
caggaucucauuccgugua

SEQ ID NO: 103 - RNA, 19 nt, Artificial Sequence
  Synthetic
gacagagauugcuuuaagu

SEQ ID NO: 104 - DNA, 19 bp, Artificial Sequence
  Synthetic
gacagagattgctttaagt

SEQ ID NO: 105 - RNA, 19 nt, Artificial Sequence
  Synthetic
cugucucuaacgaaauuca

SEQ ID NO: 106 - RNA, 19 nt, Artificial Sequence
  Synthetic
ggcugguuuauuguacugu

SEQ ID NO: 107 - DNA, 19 bp, Artificial Sequence
  Synthetic
ggctggtttattgtactgt

SEQ ID NO: 108 - RNA, 19 nt, Artificial Sequence
  Synthetic
ccgaccaaauaacaugaca

SEQ ID NO: 109 - RNA, 19 nt, Artificial Sequence
  Synthetic
gagagccacuucagaagaa

SEQ ID NO: 110 - DNA, 19 bp, Artificial Sequence
  Synthetic
gagagccacttcagaagaa

SEQ ID NO: 111 - RNA, 19 nt, Artificial Sequence
  Synthetic
cucucggugaagucuucuu

SEQ ID NO: 112 - RNA, 19 nt, Artificial Sequence
  Synthetic
ccuggcuuaucucucagaa

SEQ ID NO: 113 - DNA, 19 bp, Artificial Sequence
  Synthetic
cctggcttatctctcagaa

SEQ ID NO: 114 - RNA, 19 nt, Artificial Sequence
  Synthetic
ggaccgaauagagagucuu

SEQ ID NO: 115 - RNA, 19 nt, Artificial Sequence
  Synthetic
gagaagugauuccuguaau

SEQ ID NO: 116 - DNA, 19 bp, Artificial Sequence
  Synthetic
gagaagtgattcctgtaat

SEQ ID NO: 117 - RNA, 19 nt, Artificial Sequence
  Synthetic
cucuucacuaaggacauua

SEQ ID NO: 118 - RNA, 19 nt, Artificial Sequence
  Synthetic
guaccugcuuuggcaauca

SEQ ID NO: 119 - DNA, 19 bp, Artificial Sequence
  Synthetic
gtacctgctttggcaatca

SEQ ID NO: 120 - RNA, 19 nt, Artificial Sequence
  Synthetic
cauggacgaaaccguuagu

SEQ ID NO: 121 - RNA, 19 nt, Artificial Sequence
  Synthetic
ccuggaucauacuccagaa

SEQ ID NO: 122 - DNA, 19 bp, Artificial Sequence
  Synthetic
cctggatcatactccagaa

SEQ ID NO: 123 - RNA, 19 nt, Artificial Sequence
  Synthetic
ggaccuaguaugaggucuu

SEQ ID NO: 124 - RNA, 19 nt, Artificial Sequence
  Synthetic
gggucagaguauuauucca

SEQ ID NO: 125 - DNA, 19 bp, Artificial Sequence
  Synthetic
gggtcagagtattattcca

SEQ ID NO: 126 - RNA, 19 nt, Artificial Sequence
  Synthetic
cccagucucauaauaaggu

SEQ ID NO: 127 - RNA, 19 nt, Artificial Sequence
  Synthetic
ggaacaaguucagauuuca

SEQ ID NO: 128 - DNA, 19 bp, Artificial Sequence
  Synthetic
ggaacaagttcagatttca

SEQ ID NO: 129 - RNA, 19 nt, Artificial Sequence
  Synthetic
ccuuguucaagucuaaagu

SEQ ID NO: 130 - RNA, 19 nt, Artificial Sequence
  Synthetic
cugguuuauuguacuguua

SEQ ID NO: 131 - DNA, 19 bp, Artificial Sequence
  Synthetic
ctggtttattgtactgtta

SEQ ID NO: 132 - RNA, 19 nt, Artificial Sequence
  Synthetic
gaccaaauaacaugacaau

SEQ ID NO: 133 - RNA, 19 nt, Artificial Sequence
  Synthetic
ggaugagcuuuagaaagaa

SEQ ID NO: 134 - DNA, 19 bp, Artificial Sequence
  Synthetic
ggatgagctttagaaagaa

SEQ ID NO: 135 - RNA, 19 nt, Artificial Sequence
  Synthetic
ccuacucgaaaucuuucuu

SEQ ID NO: 136 - RNA, 19 nt, Artificial Sequence
  Synthetic
ccagaagauuaucuuagaa

SEQ ID NO: 137 - DNA, 19 bp, Artificial Sequence
  Synthetic
ccagaagattatcttagaa

SEQ ID NO: 138 - RNA, 19 nt, Artificial Sequence
  Synthetic
ggucuucuaauagaaucuu

SEQ ID NO: 139 - RNA, 19 nt, Artificial Sequence
  Synthetic
gucaggugcaucauuacau

SEQ ID NO: 140 - DNA, 19 bp, Artificial Sequence
  Synthetic
gtcaggtgcatcattacat

SEQ ID NO: 141 - RNA, 19 nt, Artificial Sequence
  Synthetic
caguccacguaguaaugua

SEQ ID NO: 142 - RNA, 19 nt, Artificial Sequence
  Synthetic
gaaugugaaaggucauaau

SEQ ID NO: 143 - DNA, 19 bp, Artificial Sequence
  Synthetic
gaatgtgaaaggtcataat

SEQ ID NO: 144 - RNA, 19 nt, Artificial Sequence
  Synthetic
cuuacacuuuccaguauua

SEQ ID NO: 145 - RNA, 19 nt, Artificial Sequence
  Synthetic
gaacauaggaugagcuuua

SEQ ID NO: 146 - DNA, 19 bp, Artificial Sequence
  Synthetic
gaacataggatgagcttta

SEQ ID NO: 147 - RNA, 19 nt, Artificial Sequence
  Synthetic
cuuguauccuacucgaaau

SEQ ID NO: 148 - RNA, 19 nt, Artificial Sequence
  Synthetic
gaugucaggugcaucauua

SEQ ID NO: 149 - DNA, 19 bp, Artificial Sequence
  Synthetic
gatgtcaggtgcatcatta

SEQ ID NO: 150 - RNA, 19 nt, Artificial Sequence
  Synthetic
cuacaguccacguaguaau

SEQ ID NO: 151 - RNA, 19 nt, Artificial Sequence
  Synthetic
cagcacauauggacuauca

SEQ ID NO: 152 - DNA, 19 bp, Artificial Sequence
  Synthetic
cagcacatatggactatca

SEQ ID NO: 153 - RNA, 19 nt, Artificial Sequence
  Synthetic
gucguguauaccugauagu

SEQ ID NO: 154 - RNA, 19 nt, Artificial Sequence
  Synthetic
ccuacuuuguggauuuagu

SEQ ID NO: 155 - DNA, 19 bp, Artificial Sequence
  Synthetic
cctactttgtggatttagt

SEQ ID NO: 156 - RNA, 19 nt, Artificial Sequence
  Synthetic
ggaugaaacaccuaaauca

SEQ ID NO: 157 - RNA, 19 nt, Artificial Sequence
  Synthetic
gcauguguaaacauuguua

SEQ ID NO: 158 - DNA, 19 bp, Artificial Sequence
  Synthetic
gcatgtgtaaacattgtta

SEQ ID NO: 159 - RNA, 19 nt, Artificial Sequence
  Synthetic
cguacacauuuguaacaau

SEQ ID NO: 160 - RNA, 19 nt, Artificial Sequence
  Synthetic
guguguugauagauuaaca

SEQ ID NO: 161 - DNA, 19 bp, Artificial Sequence
  Synthetic
gtgtgttgatagattaaca

SEQ ID NO: 162 - RNA, 19 nt, Artificial Sequence
  Synthetic
cacacaacuaucuaauugu

SEQ ID NO: 163 - RNA, 19 nt, Artificial Sequence
  Synthetic
cagagggcgaucuuaacau

SEQ ID NO: 164 - DNA, 19 bp, Artificial Sequence
  Synthetic
cagagggcgatcttaacat

SEQ ID NO: 165 - RNA, 19 nt, Artificial Sequence
  Synthetic
gucucccgcuagaauugua

SEQ ID NO: 166 - RNA, 19 nt, Artificial Sequence
  Synthetic
guguguagaauuacuguaa

SEQ ID NO: 167 - DNA, 19 bp, Artificial Sequence
  Synthetic
gtgtgtagaattactgtaa

SEQ ID NO: 168 - RNA, 19 nt, Artificial Sequence
  Synthetic
cacacaucuuaaugacauu

SEQ ID NO: 169 - RNA, 19 nt, Artificial Sequence
  Synthetic
cugggacaauauucuuggu

SEQ ID NO: 170 - DNA, 19 bp, Artificial Sequence
  Synthetic
ctgggacaatattcttggt

SEQ ID NO: 171 - RNA, 19 nt, Artificial Sequence
  Synthetic
gacccuguuauaagaacca

SEQ ID NO: 172 - RNA, 19 nt, Artificial Sequence
  Synthetic
cccacuucauagagugugu

SEQ ID NO: 173 - DNA, 19 bp, Artificial Sequence
  Synthetic
cccacttcatagagtgtgt

SEQ ID NO: 174 - RNA, 19 nt, Artificial Sequence
  Synthetic
gggugaaguaucucacaca

SEQ ID NO: 175 - RNA, 19 nt, Artificial Sequence
  Synthetic
cugggauucagucuguaga

SEQ ID NO: 176 - DNA, 19 bp, Artificial Sequence
  Synthetic
ctgggattcagtctgtaga

SEQ ID NO: 177 - RNA, 19 nt, Artificial Sequence
  Synthetic
gacccuaagucagacaucu

SEQ ID NO: 178 - RNA, 19 nt, Artificial Sequence
  Synthetic
gguguguagaauuacugua

SEQ ID NO: 179 - DNA, 19 bp, Artificial Sequence
  Synthetic
ggtgtgtagaattactgta

SEQ ID NO: 180 - RNA, 19 nt, Artificial Sequence
  Synthetic
ccacacaucuuaaugacau

SEQ ID NO: 181 - RNA, 19 nt, Artificial Sequence
  Synthetic
gaguggugcuauagaugua

SEQ ID NO: 182 - DNA, 19 bp, Artificial Sequence
  Synthetic
gagtggtgctatagatgta

SEQ ID NO: 183 - RNA, 19 nt, Artificial Sequence
  Synthetic
cucaccacgauaucuacau

SEQ ID NO: 184 - RNA, 19 nt, Artificial Sequence
  Synthetic
guacuguuauacagaaugu

SEQ ID NO: 185 - DNA, 19 bp, Artificial Sequence
  Synthetic
gtactgttatacagaatgt

SEQ ID NO: 186 - RNA, 19 nt, Artificial Sequence
  Synthetic
caugacaauaugucuuaca

SEQ ID NO: 187 - RNA, 19 nt, Artificial Sequence
  Synthetic
cagcguagauacaugagau

SEQ ID NO: 188 - DNA, 19 bp, Artificial Sequence
  Synthetic
cagcgtagatacatgagat

SEQ ID NO: 189 - RNA, 19 nt, Artificial Sequence
  Synthetic
gucgcaucuauguacucua

SEQ ID NO: 190 - RNA, 19 nt, Artificial Sequence
  Synthetic
gucagaguauuauuccaau

SEQ ID NO: 191 - DNA, 19 bp, Artificial Sequence
  Synthetic
gtcagagtattattccaat

SEQ ID NO: 192 - RNA, 19 nt, Artificial Sequence
  Synthetic
cagucucauaauaagguua

SEQ ID NO: 193 - RNA, 19 nt, Artificial Sequence
  Synthetic
cacagagagaauggaagau

SEQ ID NO: 194 - DNA, 19 bp, Artificial Sequence
  Synthetic
cacagagagaatggaagat

SEQ ID NO: 195 - RNA, 19 nt, Artificial Sequence
  Synthetic
gugucucucuuaccuucua

SEQ ID NO: 196 - RNA, 19 nt, Artificial Sequence
  Synthetic
ccaucaugaaucagaaaga

SEQ ID NO: 197 - DNA, 19 bp, Artificial Sequence
  Synthetic
ccatcatgaatcagaaaga

SEQ ID NO: 198 - RNA, 19 nt, Artificial Sequence
  Synthetic
gguaguacuuagucuuucu

SEQ ID NO: 199 - RNA, 19 nt, Artificial Sequence
  Synthetic
cacacucuaaauggagaaa

SEQ ID NO: 200 - DNA, 19 bp, Artificial Sequence
  Synthetic
cacactctaaatggagaaa

SEQ ID NO: 201 - RNA, 19 nt, Artificial Sequence
  Synthetic
gugugagauuuaccucuuu

SEQ ID NO: 202 - RNA, 19 nt, Artificial Sequence
  Synthetic
guaaguuguacagugaaau

SEQ ID NO: 203 - DNA, 19 bp, Artificial Sequence
  Synthetic
gtaagttgtacagtgaaat

SEQ ID NO: 204 - RNA, 19 nt, Artificial Sequence
  Synthetic
cauucaacaugucacuuua

SEQ ID NO: 205 - RNA, 19 nt, Artificial Sequence
  Synthetic
gcaagaacgagauguucua

SEQ ID NO: 206 - DNA, 19 bp, Artificial Sequence
  Synthetic
gcaagaacgagatgttcta

SEQ ID NO: 207 - RNA, 19 nt, Artificial Sequence
  Synthetic
cguucuugcucuacaagau

SEQ ID NO: 208 - RNA, 19 nt, Artificial Sequence
  Synthetic
ccacuucagaagaagacau

SEQ ID NO: 209 - DNA, 19 bp, Artificial Sequence
  Synthetic
ccacttcagaagaagacat

SEQ ID NO: 210 - RNA, 19 nt, Artificial Sequence
  Synthetic
ggugaagucuucuucugua

SEQ ID NO: 211 - RNA, 19 nt, Artificial Sequence
  Synthetic
cagugauggagaaauaacu

SEQ ID NO: 212 - DNA, 19 bp, Artificial Sequence
  Synthetic
cagtgatggagaaataact

SEQ ID NO: 213 - RNA, 19 nt, Artificial Sequence
  Synthetic
gucacuaccucuuuauuga

SEQ ID NO: 214 - RNA, 19 nt, Artificial Sequence
  Synthetic
cacugacgaaagcuuuacu

SEQ ID NO: 215 - DNA, 19 bp, Artificial Sequence
  Synthetic
cactgacgaaagctttact

SEQ ID NO: 216 - RNA, 19 nt, Artificial Sequence
  Synthetic
gugacugcuuucgaaauga

SEQ ID NO: 217 - RNA, 19 nt, Artificial Sequence
  Synthetic
gaccuuucuacacuagugu

SEQ ID NO: 218 - DNA, 19 bp, Artificial Sequence
  Synthetic
gacctttctacactagtgt

SEQ ID NO: 219 - RNA, 19 nt, Artificial Sequence
  Synthetic
cuggaaagaugugaucaca

SEQ ID NO: 220 - RNA, 19 nt, Artificial Sequence
  Synthetic
cagagacacucuagugaaa

SEQ ID NO: 221 - DNA, 19 bp, Artificial Sequence
  Synthetic
cagagacactctagtgaaa

SEQ ID NO: 222 - RNA, 19 nt, Artificial Sequence
  Synthetic
gucucugugagaucacuuu

SEQ ID NO: 223 - RNA, 19 nt, Artificial Sequence
  Synthetic
cucucagcaauugcaguua

SEQ ID NO: 224 - DNA, 19 bp, Artificial Sequence
  Synthetic
ctctcagcaattgcagtta

SEQ ID NO: 225 - RNA, 19 nt, Artificial Sequence
  Synthetic
gagagucguuaacgucaau

SEQ ID NO: 226 - RNA, 19 nt, Artificial Sequence
  Synthetic
gcauaaggaaagacaagaa

SEQ ID NO: 227 - DNA, 19 bp, Artificial Sequence
  Synthetic
gcataaggaaagacaagaa

SEQ ID NO: 228 - RNA, 19 nt, Artificial Sequence
  Synthetic
cguauuccuuucuguucuu

SEQ ID NO: 229 - RNA, 19 nt, Artificial Sequence
  Synthetic
guugcuguuugccugcaau

SEQ ID NO: 230 - DNA, 19 bp, Artificial Sequence
  Synthetic
gttgctgtttgcctgcaat

SEQ ID NO: 231 - RNA, 19 nt, Artificial Sequence
  Synthetic
caacgacaaacggacguua

SEQ ID NO: 232 - RNA, 19 nt, Artificial Sequence
  Synthetic
cagguuaugugaagcagaa

SEQ ID NO: 233 - DNA, 19 bp, Artificial Sequence
  Synthetic
caggttatgtgaagcagaa

SEQ ID NO: 234 - RNA, 19 nt, Artificial Sequence
  Synthetic
guccaauacacuucgucuu

SEQ ID NO: 235 - RNA, 19 nt, Artificial Sequence
  Synthetic
gaugagcuuuagaaagaaa

SEQ ID NO: 236 - DNA, 19 bp, Artificial Sequence
  Synthetic
gatgagctttagaaagaaa

SEQ ID NO: 237 - RNA, 19 nt, Artificial Sequence
  Synthetic
cuacucgaaaucuuucuuu

SEQ ID NO: 238 - RNA, 19 nt, Artificial Sequence
  Synthetic
cacccaccaaauuuacaca

SEQ ID NO: 239 - DNA, 19 bp, Artificial Sequence
  Synthetic
cacccaccaaatttacaca

SEQ ID NO: 240 - RNA, 19 nt, Artificial Sequence
  Synthetic
gugggugguuuaaaugugu

SEQ ID NO: 241 - RNA, 19 nt, Artificial Sequence
  Synthetic
gaaagggauaaagguaaua

SEQ ID NO: 242 - DNA, 19 bp, Artificial Sequence
  Synthetic
gaaagggataaaggtaata

SEQ ID NO: 243 - RNA, 19 nt, Artificial Sequence
  Synthetic
cuuucccuauuuccauuau

SEQ ID NO: 244 - RNA, 19 nt, Artificial Sequence
  Synthetic
cuuccacagacagaacuua

SEQ ID NO: 245 - DNA, 19 bp, Artificial Sequence
  Synthetic
cttccacagacagaactta

SEQ ID NO: 246 - RNA, 19 nt, Artificial Sequence
  Synthetic
gaaggugucugucuugaau

SEQ ID NO: 247 - RNA, 19 nt, Artificial Sequence
  Synthetic
guuguacagugaaauaagu

SEQ ID NO: 248 - DNA, 19 bp, Artificial Sequence
  Synthetic
gttgtacagtgaaataagt

SEQ ID NO: 249 - RNA, 19 nt, Artificial Sequence
  Synthetic
caacaugucacuuuauuca

SEQ ID NO: 250 - RNA, 19 nt, Artificial Sequence
  Synthetic
ccacacucuaaauggagaa

SEQ ID NO: 251 - DNA, 19 bp, Artificial Sequence
  Synthetic
ccacactctaaatggagaa

SEQ ID NO: 252 - RNA, 19 nt, Artificial Sequence
  Synthetic
ggugugagauuuaccucuu

SEQ ID NO: 253 - RNA, 19 nt, Artificial Sequence
  Synthetic
caggauacgaucaucuaca

SEQ ID NO: 254 - DNA, 19 bp, Artificial Sequence
  Synthetic
caggatacgatcatctaca

SEQ ID NO: 255 - RNA, 19 nt, Artificial Sequence
  Synthetic
guccuaugcuaguagaugu

SEQ ID NO: 256 - RNA, 19 nt, Artificial Sequence
  Synthetic
cuaccucccacuucauaga

SEQ ID NO: 257 - DNA, 19 bp, Artificial Sequence
  Synthetic
ctacctcccacttcataga

SEQ ID NO: 258 - RNA, 19 nt, Artificial Sequence
  Synthetic
gauggagggugaaguaucu

SEQ ID NO: 259 - RNA, 19 nt, Artificial Sequence
  Synthetic
ggucagaguauuauuccaa

SEQ ID NO: 260 - DNA, 19 bp, Artificial Sequence
  Synthetic
ggtcagagtattattccaa

SEQ ID NO: 261 - RNA, 19 nt, Artificial Sequence
  Synthetic
ccagucucauaauaagguu

SEQ ID NO: 262 - RNA, 19 nt, Artificial Sequence
  Synthetic
gugugcaagaacgagaugu

SEQ ID NO: 263 - DNA, 19 bp, Artificial Sequence
  Synthetic
gtgtgcaagaacgagatgt

SEQ ID NO: 264 - RNA, 19 nt, Artificial Sequence
  Synthetic
cacacguucuugcucuaca

SEQ ID NO: 265 - RNA, 19 nt, Artificial Sequence
  Synthetic
gacaaccacugaacuagau

SEQ ID NO: 266 - DNA, 19 bp, Artificial Sequence
  Synthetic
gacaaccactgaactagat

SEQ ID NO: 267 - RNA, 19 nt, Artificial Sequence
  Synthetic
cuguuggugacuugaucua

SEQ ID NO: 268 - RNA, 19 nt, Artificial Sequence
  Synthetic
guggaugucaauacuguga

SEQ ID NO: 269 - DNA, 19 bp, Artificial Sequence
  Synthetic
gtggatgtcaatactgtga

SEQ ID NO: 270 - RNA, 19 nt, Artificial Sequence
  Synthetic
caccuacaguuaugacacu

SEQ ID NO: 271 - RNA, 19 nt, Artificial Sequence
  Synthetic
caucaugaaucagaaagau

SEQ ID NO: 272 - DNA, 19 bp, Artificial Sequence
  Synthetic
catcatgaatcagaaagat

SEQ ID NO: 273 - RNA, 19 nt, Artificial Sequence
  Synthetic
guaguacuuagucuuucua

SEQ ID NO: 274 - RNA, 19 nt, Artificial Sequence
  Synthetic
ccuauuccaucacaaucau

SEQ ID NO: 275 - DNA, 19 bp, Artificial Sequence
  Synthetic
cctattccatcacaatcat

SEQ ID NO: 276 - RNA, 19 nt, Artificial Sequence
  Synthetic
ggauaagguaguguuagua

SEQ ID NO: 277 - RNA, 19 nt, Artificial Sequence
  Synthetic
cuacacuagugugcaagaa

SEQ ID NO: 278 - DNA, 19 bp, Artificial Sequence
  Synthetic
ctacactagtgtgcaagaa

SEQ ID NO: 279 - RNA, 19 nt, Artificial Sequence
  Synthetic
gaugugaucacacguucuu

SEQ ID NO: 280 - RNA, 19 nt, Artificial Sequence
  Synthetic
gcuuucaucuaugaaauca

SEQ ID NO: 281 - DNA, 19 bp, Artificial Sequence
  Synthetic
gctttcatctatgaaatca

SEQ ID NO: 282 - RNA, 19 nt, Artificial Sequence
  Synthetic
cgaaaguagauacuuuagu

SEQ ID NO: 283 - RNA, 19 nt, Artificial Sequence
  Synthetic
gcuuucccaucaugaauca

SEQ ID NO: 284 - DNA, 19 bp, Artificial Sequence
  Synthetic
gctttcccatcatgaatca

SEQ ID NO: 285 - RNA, 19 nt, Artificial Sequence
  Synthetic
cgaaaggguaguacuuagu

SEQ ID NO: 286 - RNA, 19 nt, Artificial Sequence
  Synthetic
caugauucaugguuuacau

SEQ ID NO: 287 - DNA, 19 bp, Artificial Sequence
  Synthetic
catgattcatggtttacat

SEQ ID NO: 288 - RNA, 19 nt, Artificial Sequence
  Synthetic
guacuaaguaccaaaugua

SEQ ID NO: 289 - RNA, 19 nt, Artificial Sequence
  Synthetic
ggaagaccuuucuacacua

SEQ ID NO: 290 - DNA, 19 bp, Artificial Sequence
  Synthetic
ggaagacctttctacacta

SEQ ID NO: 291 - RNA, 19 nt, Artificial Sequence
  Synthetic
ccuucuggaaagaugugau

SEQ ID NO: 292 - RNA, 19 nt, Artificial Sequence
  Synthetic
cucuucggaaccugaagau

SEQ ID NO: 293 - DNA, 19 bp, Artificial Sequence
  Synthetic
ctcttcggaacctgaagat

SEQ ID NO: 294 - RNA, 19 nt, Artificial Sequence
  Synthetic
gagaagccuuggacuucua

SEQ ID NO: 295 - RNA, 19 nt, Artificial Sequence
  Synthetic
cacacauagauguggaugu

SEQ ID NO: 296 - DNA, 19 bp, Artificial Sequence
  Synthetic
cacacatagatgtggatgt

SEQ ID NO: 297 - RNA, 19 nt, Artificial Sequence
  Synthetic
guguguaucuacaccuaca

SEQ ID NO: 298 - RNA, 19 nt, Artificial Sequence
  Synthetic
cuccagguuaugugaagca

SEQ ID NO: 299 - DNA, 19 bp, Artificial Sequence
  Synthetic
ctccaggttatgtgaagca

SEQ ID NO: 300 - RNA, 19 nt, Artificial Sequence
  Synthetic
gagguccaauacacuucgu

SEQ ID NO: 301 - RNA, 19 nt, Artificial Sequence
  Synthetic
guucuugcuauuguugaua

SEQ ID NO: 302 - DNA, 19 bp, Artificial Sequence
  Synthetic
gttcttgctattgttgata

SEQ ID NO: 303 - RNA, 19 nt, Artificial Sequence
  Synthetic
caagaacgauaacaacuau

SEQ ID NO: 304 - RNA, 19 nt, Artificial Sequence
  Synthetic
gaacugcuuucaucuauga

SEQ ID NO: 305 - DNA, 19 bp, Artificial Sequence
  Synthetic
gaactgctttcatctatga

SEQ ID NO: 306 - RNA, 19 nt, Artificial Sequence
  Synthetic
cuugacgaaaguagauacu

SEQ ID NO: 307 - RNA, 19 nt, Artificial Sequence
  Synthetic
ggaagaauauggaugcaua

SEQ ID NO: 308 - DNA, 19 bp, Artificial Sequence
  Synthetic
ggaagaatatggatgcata

SEQ ID NO: 309 - RNA, 19 nt, Artificial Sequence
  Synthetic
ccuucuuauaccuacguau

SEQ ID NO: 310 - RNA, 19 nt, Artificial Sequence
  Synthetic
ggacaauauucuugguccu

SEQ ID NO: 311 - DNA, 19 bp, Artificial Sequence
  Synthetic
ggacaatattcttggtcct

SEQ ID NO: 312 - RNA, 19 nt, Artificial Sequence
  Synthetic
ccuguuauaagaaccagga

SEQ ID NO: 313 - RNA, 19 nt, Artificial Sequence
  Synthetic
guguuccugaagaaauaga

SEQ ID NO: 314 - DNA, 19 bp, Artificial Sequence
  Synthetic
gtgttcctgaagaaataga

SEQ ID NO: 315 - RNA, 19 nt, Artificial Sequence
  Synthetic
cacaaggacuucuuuaucu

SEQ ID NO: 316 - RNA, 19 nt, Artificial Sequence
  Synthetic
cgaaugugaaaggucauaa

SEQ ID NO: 317 - DNA, 19 bp, Artificial Sequence
  Synthetic
cgaatgtgaaaggtcataa

SEQ ID NO: 318 - RNA, 19 nt, Artificial Sequence
  Synthetic
gcuuacacuuuccaguauu

SEQ ID NO: 319 - RNA, 19 nt, Artificial Sequence
  Synthetic
gugagcuugaacauaggau

SEQ ID NO: 320 - DNA, 19 bp, Artificial Sequence
  Synthetic
gtgagcttgaacataggat

SEQ ID NO: 321 - RNA, 19 nt, Artificial Sequence
  Synthetic
cacucgaacuuguauccua

SEQ ID NO: 322 - RNA, 19 nt, Artificial Sequence
  Synthetic
cugauacaguacucaauga

SEQ ID NO: 323 - DNA, 19 bp, Artificial Sequence
  Synthetic
ctgatacagtactcaatga

SEQ ID NO: 324 - RNA, 19 nt, Artificial Sequence
  Synthetic
gacuaugucaugaguuacu

SEQ ID NO: 325 - RNA, 19 nt, Artificial Sequence
  Synthetic
gcaauaggcuauaaggaau

SEQ ID NO: 326 - DNA, 19 bp, Artificial Sequence
  Synthetic
gcaataggctataaggaat

SEQ ID NO: 327 - RNA, 19 nt, Artificial Sequence
  Synthetic
cguuauccgauauuccuua

SEQ ID NO: 328 - RNA, 19 nt, Artificial Sequence
  Synthetic
cuucugcaaucaacugaaa

SEQ ID NO: 329 - DNA, 19 bp, Artificial Sequence
  Synthetic
cttctgcaatcaactgaaa

SEQ ID NO: 330 - RNA, 19 nt, Artificial Sequence
  Synthetic
gaagacguuaguugacuuu

SEQ ID NO: 331 - RNA, 19 nt, Artificial Sequence
  Synthetic
caugaucgcugguaaagua

SEQ ID NO: 332 - DNA, 19 bp, Artificial Sequence
  Synthetic
catgatcgctggtaaagta

SEQ ID NO: 333 - RNA, 19 nt, Artificial Sequence
  Synthetic
guacuagcgaccauuucau

SEQ ID NO: 334 - RNA, 19 nt, Artificial Sequence
  Synthetic
gauguggacagcuugaugu

SEQ ID NO: 335 - DNA, 19 bp, Artificial Sequence
  Synthetic
gatgtggacagcttgatgt

SEQ ID NO: 336 - RNA, 19 nt, Artificial Sequence
  Synthetic
cuacaccugucgaacuaca

SEQ ID NO: 337 - RNA, 19 nt, Artificial Sequence
  Synthetic
cacacaguguuccugaaga

SEQ ID NO: 338 - DNA, 19 bp, Artificial Sequence
  Synthetic
cacacagtgttcctgaaga

SEQ ID NO: 339 - RNA, 19 nt, Artificial Sequence
  Synthetic
gugugucacaaggacuucu

SEQ ID NO: 340 - RNA, 19 nt, Artificial Sequence
  Synthetic
gauguaaacuugaccacaa

SEQ ID NO: 341 - DNA, 19 bp, Artificial Sequence
  Synthetic
gatgtaaacttgaccacaa

SEQ ID NO: 342 - RNA, 19 nt, Artificial Sequence
  Synthetic
cuacauuugaacugguguu

SEQ ID NO: 343 - RNA, 19 nt, Artificial Sequence
  Synthetic
cuccagaauucugcucuca

SEQ ID NO: 344 - DNA, 19 bp, Artificial Sequence
  Synthetic
ctccagaattctgctctca

SEQ ID NO: 345 - RNA, 19 nt, Artificial Sequence
  Synthetic
gaggucuuaagacgagagu

SEQ ID NO: 346 - RNA, 19 nt, Artificial Sequence
  Synthetic
gauagaccuugauuuaaca

SEQ ID NO: 347 - DNA, 19 bp, Artificial Sequence
  Synthetic
gatagaccttgatttaaca

SEQ ID NO: 348 - RNA, 19 nt, Artificial Sequence
  Synthetic
cuaucuggaacuaaauugu

SEQ ID NO: 349 - RNA, 19 nt, Artificial Sequence
  Synthetic
cagagagaauggaagauca

SEQ ID NO: 350 - DNA, 19 bp, Artificial Sequence
  Synthetic
cagagagaatggaagatca

SEQ ID NO: 351 - RNA, 19 nt, Artificial Sequence
  Synthetic
gucucucuuaccuucuagu

SEQ ID NO: 352 - RNA, 19 nt, Artificial Sequence
  Synthetic
cuuacugggacaauauucu

SEQ ID NO: 353 - DNA, 19 bp, Artificial Sequence
  Synthetic
cttactgggacaatattct

SEQ ID NO: 354 - RNA, 19 nt, Artificial Sequence
  Synthetic
gaaugacccuguuauaaga

SEQ ID NO: 355 - RNA, 19 nt, Artificial Sequence
  Synthetic
caaccacugaacuagauga

SEQ ID NO: 356 - DNA, 19 bp, Artificial Sequence
  Synthetic
caaccactgaactagatga

SEQ ID NO: 357 - RNA, 19 nt, Artificial Sequence
  Synthetic
guuggugacuugaucuacu

SEQ ID NO: 358 - RNA, 19 nt, Artificial Sequence
  Synthetic
gugucaaggugaaaucuga

SEQ ID NO: 359 - DNA, 19 bp, Artificial Sequence
  Synthetic
gtgtcaaggtgaaatctga

SEQ ID NO: 360 - RNA, 19 nt, Artificial Sequence
  Synthetic
cacaguuccacuuuagacu

SEQ ID NO: 361 - RNA, 19 nt, Artificial Sequence
  Synthetic
gaaagaaagugagcuugaa

SEQ ID NO: 362 - DNA, 19 bp, Artificial Sequence
  Synthetic
gaaagaaagtgagcttgaa

SEQ ID NO: 363 - RNA, 19 nt, Artificial Sequence
  Synthetic
cuuucuuucacucgaacuu

SEQ ID NO: 364 - RNA, 19 nt, Artificial Sequence
  Synthetic
cuggagaagugauuccugu

SEQ ID NO: 365 - DNA, 19 bp, Artificial Sequence
  Synthetic
ctggagaagtgattcctgt

SEQ ID NO: 366 - RNA, 19 nt, Artificial Sequence
  Synthetic
gaccucuucacuaaggaca

SEQ ID NO: 367 - RNA, 19 nt, Artificial Sequence
  Synthetic
gacaguuggaaugcaguga

SEQ ID NO: 368 - DNA, 19 bp, Artificial Sequence
  Synthetic
gacagttggaatgcagtga

SEQ ID NO: 369 - RNA, 19 nt, Artificial Sequence
  Synthetic
cugucaaccuuacgucacu

SEQ ID NO: 370 - RNA, 19 nt, Artificial Sequence
  Synthetic
ggaugcauaaggaaagaca

SEQ ID NO: 371 - DNA, 19 bp, Artificial Sequence
  Synthetic
ggatgcataaggaaagaca

SEQ ID NO: 372 - RNA, 19 nt, Artificial Sequence
  Synthetic
ccuacguauuccuuucugu

SEQ ID NO: 373 - RNA, 19 nt, Artificial Sequence
  Synthetic
cauugcaacucugagauua

SEQ ID NO: 374 - DNA, 19 bp, Artificial Sequence
  Synthetic
cattgcaactctgagatta

SEQ ID NO: 375 - RNA, 19 nt, Artificial Sequence
  Synthetic
guaacguugagacucuaau

SEQ ID NO: 376 - RNA, 19 nt, Artificial Sequence
  Synthetic
gaucgcugguaaaguagcu

SEQ ID NO: 377 - DNA, 19 bp, Artificial Sequence
  Synthetic
gatcgctggtaaagtagct

SEQ ID NO: 378 - RNA, 19 nt, Artificial Sequence
  Synthetic
cuagcgaccauuucaucga

SEQ ID NO: 379 - RNA, 19 nt, Artificial Sequence
  Synthetic
cacacacauagauguggau

SEQ ID NO: 380 - DNA, 19 bp, Artificial Sequence
  Synthetic
cacacacatagatgtggat

SEQ ID NO: 381 - RNA, 19 nt, Artificial Sequence
  Synthetic
guguguguaucuacaccua

SEQ ID NO: 382 - RNA, 19 nt, Artificial Sequence
  Synthetic
ccuucgaaaugcagagagu

SEQ ID NO: 383 - DNA, 19 bp, Artificial Sequence
  Synthetic
ccttcgaaatgcagagagt

SEQ ID NO: 384 - RNA, 19 nt, Artificial Sequence
  Synthetic
ggaagcuuuacgucucuca

SEQ ID NO: 385 - RNA, 19 nt, Artificial Sequence
  Synthetic
cacuacaguucucacaaga

SEQ ID NO: 386 - DNA, 19 bp, Artificial Sequence
  Synthetic
cactacagttctcacaaga

SEQ ID NO: 387 - RNA, 19 nt, Artificial Sequence
  Synthetic
gugaugucaagaguguucu

SEQ ID NO: 388 - RNA, 19 nt, Artificial Sequence
  Synthetic
cacugaacuagaugacugu

SEQ ID NO: 389 - DNA, 19 bp, Artificial Sequence
  Synthetic
cactgaactagatgactgt

SEQ ID NO: 390 - RNA, 19 nt, Artificial Sequence
  Synthetic
gugacuugaucuacugaca

SEQ ID NO: 391 - RNA, 19 nt, Artificial Sequence
  Synthetic
ggugaaaucugaguuggcu

SEQ ID NO: 392 - DNA, 19 bp, Artificial Sequence
  Synthetic
ggtgaaatctgagttggct

SEQ ID NO: 393 - RNA, 19 nt, Artificial Sequence
  Synthetic
ccacuuuagacucaaccga

SEQ ID NO: 394 - RNA, 19 nt, Artificial Sequence
  Synthetic
cggaaaggaagaauaugga

SEQ ID NO: 395 - DNA, 19 bp, Artificial Sequence
  Synthetic
cggaaaggaagaatatgga

SEQ ID NO: 396 - RNA, 19 nt, Artificial Sequence
  Synthetic
gccuuuccuucuuauaccu

SEQ ID NO: 397 - RNA, 19 nt, Artificial Sequence
  Synthetic
gugcaucauuacauugggu

SEQ ID NO: 398 - DNA, 19 bp, Artificial Sequence
  Synthetic
gtgcatcattacattgggt

SEQ ID NO: 399 - RNA, 19 nt, Artificial Sequence
  Synthetic
cacguaguaauguaaccca

SEQ ID NO: 400 - RNA, 19 nt, Artificial Sequence
  Synthetic
cuguugccaagacagagau

SEQ ID NO: 401 - DNA, 19 bp, Artificial Sequence
  Synthetic
ctgttgccaagacagagat

SEQ ID NO: 402 - RNA, 19 nt, Artificial Sequence
  Synthetic
gacaacgguucugucucua

SEQ ID NO: 403 - RNA, 19 nt, Artificial Sequence
  Synthetic
cauuguaguuacacaaaca

SEQ ID NO: 404 - DNA, 19 bp, Artificial Sequence
  Synthetic
cattgtagttacacaaaca

SEQ ID NO: 405 - RNA, 19 nt, Artificial Sequence
  Synthetic
guaacaucaauguguuugu

SEQ ID NO: 406 - RNA, 19 nt, Artificial Sequence
  Synthetic
ggaaccugaagauagaccu

SEQ ID NO: 407 - DNA, 19 bp, Artificial Sequence
  Synthetic
ggaacctgaagatagacct

SEQ ID NO: 408 - RNA, 19 nt, Artificial Sequence
  Synthetic
ccuuggacuucuaucugga

SEQ ID NO: 409 - RNA, 19 nt, Artificial Sequence
  Synthetic
gucaaggugaaaucugagu

SEQ ID NO: 410 - DNA, 19 bp, Artificial Sequence
  Synthetic
gtcaaggtgaaatctgagt

SEQ ID NO: 411 - RNA, 19 nt, Artificial Sequence
  Synthetic
caguuccacuuuagacuca

SEQ ID NO: 412 - RNA, 19 nt, Artificial Sequence
  Synthetic
cauaggaugagcuuuagaa

SEQ ID NO: 413 - DNA, 19 bp, Artificial Sequence
  Synthetic
cataggatgagctttagaa

SEQ ID NO: 414 - RNA, 19 nt, Artificial Sequence
  Synthetic
guauccuacucgaaaucuu

SEQ ID NO: 415 - RNA, 19 nt, Artificial Sequence
  Synthetic
caguacucaaugaugauga

SEQ ID NO: 416 - DNA, 19 bp, Artificial Sequence
  Synthetic
cagtactcaatgatgatga

SEQ ID NO: 417 - RNA, 19 nt, Artificial Sequence
  Synthetic
gucaugaguuacuacuacu

SEQ ID NO: 418 - RNA, 19 nt, Artificial Sequence
  Synthetic
caauaggcuauaaggaaua

SEQ ID NO: 419 - DNA, 19 bp, Artificial Sequence
  Synthetic
caataggctataaggaata

SEQ ID NO: 420 - RNA, 19 nt, Artificial Sequence
  Synthetic
guuauccgauauuccuuau

SEQ ID NO: 421 - RNA, 19 nt, Artificial Sequence
  Synthetic
ccacacacauagaugugga

SEQ ID NO: 422 - DNA, 19 bp, Artificial Sequence
  Synthetic
ccacacacatagatgtgga

SEQ ID NO: 423 - RNA, 19 nt, Artificial Sequence
  Synthetic
gguguguguaucuacaccu

SEQ ID NO: 424 - RNA, 19 nt, Artificial Sequence
  Synthetic
caaccacacucuaaaugga

SEQ ID NO: 425 - DNA, 19 bp, Artificial Sequence
  Synthetic
caaccacactctaaatgga

SEQ ID NO: 426 - RNA, 19 nt, Artificial Sequence
  Synthetic
guuggugugagauuuaccu

SEQ ID NO: 427 - RNA, 19 nt, Artificial Sequence
  Synthetic
gauugcuuuaaguggcaaa

SEQ ID NO: 428 - DNA, 19 bp, Artificial Sequence
  Synthetic
gattgctttaagtggcaaa

SEQ ID NO: 429 - RNA, 19 nt, Artificial Sequence
  Synthetic
cuaacgaaauucaccguuu

SEQ ID NO: 430 - RNA, 19 nt, Artificial Sequence
  Synthetic
gaugaugauauuggugaca

SEQ ID NO: 431 - DNA, 19 bp, Artificial Sequence
  Synthetic
gatgatgatattggtgaca

SEQ ID NO: 432 - RNA, 19 nt, Artificial Sequence
  Synthetic
cuacuacuauaaccacugu

SEQ ID NO: 433 - RNA, 19 nt, Artificial Sequence
  Synthetic
cauagaguguguguugaua

SEQ ID NO: 434 - DNA, 19 bp, Artificial Sequence
  Synthetic
catagagtgtgtgttgata

SEQ ID NO: 435 - RNA, 19 nt, Artificial Sequence
  Synthetic
guaucucacacacaacuau

SEQ ID NO: 436 - RNA, 19 nt, Artificial Sequence
  Synthetic
cucuagugaaagccuuccu

SEQ ID NO: 437 - DNA, 19 bp, Artificial Sequence
  Synthetic
ctctagtgaaagccttcct

SEQ ID NO: 438 - RNA, 19 nt, Artificial Sequence
  Synthetic
gagaucacuuucggaagga

SEQ ID NO: 439 - RNA, 19 nt, Artificial Sequence
  Synthetic
cacagagacacucuaguga

SEQ ID NO: 440 - DNA, 19 bp, Artificial Sequence
  Synthetic
cacagagacactctagtga

SEQ ID NO: 441 - RNA, 19 nt, Artificial Sequence
  Synthetic
gugucucugugagaucacu

SEQ ID NO: 442 - RNA, 19 nt, Artificial Sequence
  Synthetic
guuaugugaagcagaauca

SEQ ID NO: 443 - DNA, 19 bp, Artificial Sequence
  Synthetic
gttatgtgaagcagaatca

SEQ ID NO: 444 - RNA, 19 nt, Artificial Sequence
  Synthetic
caauacacuucgucuuagu

SEQ ID NO: 445 - RNA, 19 nt, Artificial Sequence
  Synthetic
cuaccuuguagugucccau

SEQ ID NO: 446 - DNA, 19 bp, Artificial Sequence
  Synthetic
ctaccttgtagtgtcccat

SEQ ID NO: 447 - RNA, 19 nt, Artificial Sequence
  Synthetic
gauggaacaucacagggua

SEQ ID NO: 448 - RNA, 19 nt, Artificial Sequence
  Synthetic
gauuucaauuccacagaaa

SEQ ID NO: 449 - DNA, 19 bp, Artificial Sequence
  Synthetic
gatttcaattccacagaaa

SEQ ID NO: 450 - RNA, 19 nt, Artificial Sequence
  Synthetic
cuaaaguuaaggugucuuu

SEQ ID NO: 451 - RNA, 19 nt, Artificial Sequence
  Synthetic
guuucuaccucccacuuca

SEQ ID NO: 452 - DNA, 19 bp, Artificial Sequence
  Synthetic
gtttctacctcccacttca

SEQ ID NO: 453 - RNA, 19 nt, Artificial Sequence
  Synthetic
caaagauggagggugaagu

SEQ ID NO: 454 - RNA, 19 nt, Artificial Sequence
  Synthetic
ggucauaauagcuuuccca

SEQ ID NO: 455 - DNA, 19 bp, Artificial Sequence
  Synthetic
ggtcataatagctttccca

SEQ ID NO: 456 - RNA, 19 nt, Artificial Sequence
  Synthetic
ccaguauuaucgaaagggu

SEQ ID NO: 457 - RNA, 19 nt, Artificial Sequence
  Synthetic
cuuuccggcaagucaugua

SEQ ID NO: 458 - DNA, 19 bp, Artificial Sequence
  Synthetic
ctttccggcaagtcatgta

SEQ ID NO: 459 - RNA, 19 nt, Artificial Sequence
  Synthetic
gaaaggccguucaguacau

SEQ ID NO: 460 - RNA, 19 nt, Artificial Sequence
  Synthetic
cgaaagcuuuacuccugau

SEQ ID NO: 461 - DNA, 19 bp, Artificial Sequence
  Synthetic
cgaaagctttactcctgat

SEQ ID NO: 462 - RNA, 19 nt, Artificial Sequence
  Synthetic
gcuuucgaaaugaggacua

SEQ ID NO: 463 - RNA, 19 nt, Artificial Sequence
  Synthetic
cuuuggcagagcuaaguua

SEQ ID NO: 464 - DNA, 19 bp, Artificial Sequence
  Synthetic
ctttggcagagctaagtta

SEQ ID NO: 465 - RNA, 19 nt, Artificial Sequence
  Synthetic
gaaaccgucucgauucaau

SEQ ID NO: 466 - RNA, 19 nt, Artificial Sequence
  Synthetic
gcuuuggcaaucauugcaa

SEQ ID NO: 467 - DNA, 19 bp, Artificial Sequence
  Synthetic
gctttggcaatcattgcaa

SEQ ID NO: 468 - RNA, 19 nt, Artificial Sequence
  Synthetic
cgaaaccguuaguaacguu

SEQ ID NO: 469 - RNA, 19 nt, Artificial Sequence
  Synthetic
gcauuuggauaaugugaca

SEQ ID NO: 470 - DNA, 19 bp, Artificial Sequence
  Synthetic
gcatttggataatgtgaca

SEQ ID NO: 471 - RNA, 19 nt, Artificial Sequence
  Synthetic
cguaaaccuauuacacugu

SEQ ID NO: 472 - RNA, 19 nt, Artificial Sequence
  Synthetic
guaaacuugaccacaacua

SEQ ID NO: 473 - DNA, 19 bp, Artificial Sequence
  Synthetic
gtaaacttgaccacaacta

SEQ ID NO: 474 - RNA, 19 nt, Artificial Sequence
  Synthetic
cauuugaacugguguugau

SEQ ID NO: 475 - RNA, 19 nt, Artificial Sequence
  Synthetic
cuuuacuccugauuugaau

SEQ ID NO: 476 - DNA, 19 bp, Artificial Sequence
  Synthetic
ctttactcctgatttgaat

SEQ ID NO: 477 - RNA, 19 nt, Artificial Sequence
  Synthetic
gaaaugaggacuaaacuua

SEQ ID NO: 478 - RNA, 19 nt, Artificial Sequence
  Synthetic
cccuuuaaaucucuucgga

SEQ ID NO: 479 - DNA, 19 bp, Artificial Sequence
  Synthetic
ccctttaaatctcttcgga

SEQ ID NO: 480 - RNA, 19 nt, Artificial Sequence
  Synthetic
gggaaauuuagagaagccu

SEQ ID NO: 481 - RNA, 19 nt, Artificial Sequence
  Synthetic
caguuaaguaaguuacacu

SEQ ID NO: 482 - DNA, 19 bp, Artificial Sequence
  Synthetic
cagttaagtaagttacact

SEQ ID NO: 483 - RNA, 19 nt, Artificial Sequence
  Synthetic
gucaauucauucaauguga

SEQ ID NO: 484 - RNA, 19 nt, Artificial Sequence
  Synthetic
cauaaggaaagacaagaaa

SEQ ID NO: 485 - DNA, 19 bp, Artificial Sequence
  Synthetic
cataaggaaagacaagaaa

SEQ ID NO: 486 - RNA, 19 nt, Artificial Sequence
  Synthetic
guauuccuuucuguucuuu

SEQ ID NO: 487 - RNA, 19 nt, Artificial Sequence
  Synthetic
caagucauguaugcuccau

SEQ ID NO: 488 - DNA, 19 bp, Artificial Sequence
  Synthetic
caagtcatgtatgctccat

SEQ ID NO: 489 - RNA, 19 nt, Artificial Sequence
  Synthetic
guucaguacauacgaggua

SEQ ID NO: 490 - RNA, 19 nt, Artificial Sequence
  Synthetic
cacaguuucuacuuguccu

SEQ ID NO: 491 - DNA, 19 bp, Artificial Sequence
  Synthetic
cacagtttctacttgtcct

SEQ ID NO: 492 - RNA, 19 nt, Artificial Sequence
  Synthetic
gugucaaagaugaacagga

SEQ ID NO: 493 - RNA, 19 nt, Artificial Sequence
  Synthetic
caucagcucacacuugcaa

SEQ ID NO: 494 - DNA, 19 bp, Artificial Sequence
  Synthetic
catcagctcacacttgcaa

SEQ ID NO: 495 - RNA, 19 nt, Artificial Sequence
  Synthetic
guagucgagugugaacguu

SEQ ID NO: 496 - RNA, 19 nt, Artificial Sequence
  Synthetic
cacagaaagaaagugagcu

SEQ ID NO: 497 - DNA, 19 bp, Artificial Sequence
  Synthetic
cacagaaagaaagtgagct

SEQ ID NO: 498 - RNA, 19 nt, Artificial Sequence
  Synthetic
gugucuuucuuucacucga

SEQ ID NO: 499 - RNA, 19 nt, Artificial Sequence
  Synthetic
gucuuaccauguaccugcu

SEQ ID NO: 500 - DNA, 19 bp, Artificial Sequence
  Synthetic
gtcttaccatgtacctgct

SEQ ID NO: 501 - RNA, 19 nt, Artificial Sequence
  Synthetic
cagaaugguacauggacga

SEQ ID NO: 502 - RNA, 19 nt, Artificial Sequence
  Synthetic
caauugcaguuaaguaagu

SEQ ID NO: 503 - DNA, 19 bp, Artificial Sequence
  Synthetic
caattgcagttaagtaagt

SEQ ID NO: 504 - RNA, 19 nt, Artificial Sequence
  Synthetic
guuaacgucaauucauuca

SEQ ID NO: 505 - RNA, 19 nt, Artificial Sequence
  Synthetic
cuguuccguuguaguaggu

SEQ ID NO: 506 - DNA, 19 bp, Artificial Sequence
  Synthetic
ctgttccgttgtagtaggt

SEQ ID NO: 507 - RNA, 19 nt, Artificial Sequence
  Synthetic
gacaaggcaacaucaucca

SEQ ID NO: 508 - RNA, 19 nt, Artificial Sequence
  Synthetic
cuuugauggaaacuggaau

SEQ ID NO: 509 - DNA, 19 bp, Artificial Sequence
  Synthetic
ctttgatggaaactggaat

SEQ ID NO: 510 - RNA, 19 nt, Artificial Sequence
  Synthetic
gaaacuaccuuugaccuua

SEQ ID NO: 511 - RNA, 19 nt, Artificial Sequence
  Synthetic
cagaaguacuuuccuugca

SEQ ID NO: 512 - DNA, 19 bp, Artificial Sequence
  Synthetic
cagaagtactttccttgca

SEQ ID NO: 513 - RNA, 19 nt, Artificial Sequence
  Synthetic
gucuucaugaaaggaacgu

SEQ ID NO: 514 - RNA, 19 nt, Artificial Sequence
  Synthetic
cgaucaucuacacugacga

SEQ ID NO: 515 - DNA, 19 bp, Artificial Sequence
  Synthetic
cgatcatctacactgacga

SEQ ID NO: 516 - RNA, 19 nt, Artificial Sequence
  Synthetic
gcuaguagaugugacugcu

SEQ ID NO: 517 - RNA, 19 nt, Artificial Sequence
  Synthetic
caugaaggcuuucuucuca

SEQ ID NO: 518 - DNA, 19 bp, Artificial Sequence
  Synthetic
catgaaggctttcttctca

SEQ ID NO: 519 - RNA, 19 nt, Artificial Sequence
  Synthetic
guacuuccgaaagaagagu

SEQ ID NO: 520 - RNA, 19 nt, Artificial Sequence
  Synthetic
gaauucugcucucagcaau

SEQ ID NO: 521 - DNA, 19 bp, Artificial Sequence
  Synthetic
gaattctgctctcagcaat

SEQ ID NO: 522 - RNA, 19 nt, Artificial Sequence
  Synthetic
cuuaagacgagagucguua

SEQ ID NO: 523 - RNA, 19 nt, Artificial Sequence
  Synthetic
cuuacacagagacacucua

SEQ ID NO: 524 - DNA, 19 bp, Artificial Sequence
  Synthetic
cttacacagagacactcta

SEQ ID NO: 525 - RNA, 19 nt, Artificial Sequence
  Synthetic
gaaugugucucugugagau

SEQ ID NO: 526 - RNA, 19 nt, Artificial Sequence
  Synthetic
caaucaugaucgcugguaa

SEQ ID NO: 527 - DNA, 19 bp, Artificial Sequence
  Synthetic
caatcatgatcgctggtaa

SEQ ID NO: 528 - RNA, 19 nt, Artificial Sequence
  Synthetic
guuaguacuagcgaccauu

SEQ ID NO: 529 - RNA, 19 nt, Artificial Sequence
  Synthetic
gcuauaaggaauagcagga

SEQ ID NO: 530 - DNA, 19 bp, Artificial Sequence
  Synthetic
gctataaggaatagcagga

SEQ ID NO: 531 - RNA, 19 nt, Artificial Sequence
  Synthetic
cgauauuccuuaucguccu

SEQ ID NO: 532 - RNA, 19 nt, Artificial Sequence
  Synthetic
caaagacagaacagguacu

SEQ ID NO: 533 - DNA, 19 bp, Artificial Sequence
  Synthetic
caaagacagaacaggtact

SEQ ID NO: 534 - RNA, 19 nt, Artificial Sequence
  Synthetic
guuucugucuuguccauga

SEQ ID NO: 535 - RNA, 19 nt, Artificial Sequence
  Synthetic
gcuuucuucucaaugccau

SEQ ID NO: 536 - DNA, 19 bp, Artificial Sequence
  Synthetic
gctttcttctcaatgccat

SEQ ID NO: 537 - RNA, 19 nt, Artificial Sequence
  Synthetic
cgaaagaagaguuacggua

SEQ ID NO: 538 - RNA, 19 nt, Artificial Sequence
  Synthetic
gcuaaaggauucaacugga

SEQ ID NO: 539 - DNA, 19 bp, Artificial Sequence
  Synthetic
gctaaaggattcaactgga

SEQ ID NO: 540 - RNA, 19 nt, Artificial Sequence
  Synthetic
cgauuuccuaaguugaccu

SEQ ID NO: 541 - RNA, 19 nt, Artificial Sequence
  Synthetic
cuagugugcaagaacgaga

SEQ ID NO: 542 - DNA, 19 bp, Artificial Sequence
  Synthetic
ctagtgtgcaagaacgaga

SEQ ID NO: 543 - RNA, 19 nt, Artificial Sequence
  Synthetic
gaucacacguucuugcucu

SEQ ID NO: 544 - RNA, 19 nt, Artificial Sequence
  Synthetic
gaucaggucuuucagcuga

SEQ ID NO: 545 - DNA, 19 bp, Artificial Sequence
  Synthetic
gatcaggtctttcagctga

SEQ ID NO: 546 - RNA, 19 nt, Artificial Sequence
  Synthetic
cuaguccagaaagucgacu

SEQ ID NO: 547 - RNA, 19 nt, Artificial Sequence
  Synthetic
cuaauuacuuggaacaagu

SEQ ID NO: 548 - DNA, 19 bp, Artificial Sequence
  Synthetic
ctaattacttggaacaagt

SEQ ID NO: 549 - RNA, 19 nt, Artificial Sequence
  Synthetic
gauuaaugaaccuuguuca

SEQ ID NO: 550 - RNA, 19 nt, Artificial Sequence
  Synthetic
guucgaaugugaaagguca

SEQ ID NO: 551 - DNA, 19 bp, Artificial Sequence
  Synthetic
gttcgaatgtgaaaggtca

SEQ ID NO: 552 - RNA, 19 nt, Artificial Sequence
  Synthetic
caagcuuacacuuuccagu

SEQ ID NO: 553 - RNA, 19 nt, Artificial Sequence
  Synthetic
guguaacuuaauaagccua

SEQ ID NO: 554 - DNA, 19 bp, Artificial Sequence
  Synthetic
gtgtaacttaataagccta

SEQ ID NO: 555 - RNA, 19 nt, Artificial Sequence
  Synthetic
cacauugaauuauucggau

SEQ ID NO: 556 - RNA, 19 nt, Artificial Sequence
  Synthetic
guagauacaugagauccga

SEQ ID NO: 557 - DNA, 19 bp, Artificial Sequence
  Synthetic
gtagatacatgagatccga

SEQ ID NO: 558 - RNA, 19 nt, Artificial Sequence
  Synthetic
caucuauguacucuaggcu

SEQ ID NO: 559 - RNA, 19 nt, Artificial Sequence
  Synthetic
gaaguacuuuccuugcaca

SEQ ID NO: 560 - DNA, 19 bp, Artificial Sequence
  Synthetic
gaagtactttccttgcaca

SEQ ID NO: 561 - RNA, 19 nt, Artificial Sequence
  Synthetic
cuucaugaaaggaacgugu

SEQ ID NO: 562 - RNA, 19 nt, Artificial Sequence
  Synthetic
cuauguaguugagcucugu

SEQ ID NO: 563 - DNA, 19 bp, Artificial Sequence
  Synthetic
ctatgtagttgagctctgt

SEQ ID NO: 564 - RNA, 19 nt, Artificial Sequence
  Synthetic
gauacaucaacucgagaca

SEQ ID NO: 565 - RNA, 19 nt, Artificial Sequence
  Synthetic
cguuguaguagguagcagu

SEQ ID NO: 566 - DNA, 19 bp, Artificial Sequence
  Synthetic
cgttgtagtaggtagcagt

SEQ ID NO: 567 - RNA, 19 nt, Artificial Sequence
  Synthetic
gcaacaucauccaucguca

SEQ ID NO: 568 - RNA, 19 nt, Artificial Sequence
  Synthetic
ccuucuugcauuucugccu

SEQ ID NO: 569 - DNA, 19 bp, Artificial Sequence
  Synthetic
ccttcttgcatttctgcct

SEQ ID NO: 570 - RNA, 19 nt, Artificial Sequence
  Synthetic
ggaagaacguaaagacgga

SEQ ID NO: 571 - RNA, 19 nt, Artificial Sequence
  Synthetic
guucagauuucacugguca

SEQ ID NO: 572 - DNA, 19 bp, Artificial Sequence
  Synthetic
gttcagatttcactggtca

SEQ ID NO: 573 - RNA, 19 nt, Artificial Sequence
  Synthetic
caagucuaaagugaccagu

SEQ ID NO: 574 - RNA, 19 nt, Artificial Sequence
  Synthetic
caaugaugaugauauuggu

SEQ ID NO: 575 - DNA, 19 bp, Artificial Sequence
  Synthetic
caatgatgatgatattggt

SEQ ID NO: 576 - RNA, 19 nt, Artificial Sequence
  Synthetic
guuacuacuacuauaacca

SEQ ID NO: 577 - RNA, 19 nt, Artificial Sequence
  Synthetic
cuugaacauaggaugagcu

SEQ ID NO: 578 - DNA, 19 bp, Artificial Sequence
  Synthetic
cttgaacataggatgagct

SEQ ID NO: 579 - RNA, 19 nt, Artificial Sequence
  Synthetic
gaacuuguauccuacucga

SEQ ID NO: 580 - RNA, 19 nt, Artificial Sequence
  Synthetic
gagaauggaagaucagggu

SEQ ID NO: 581 - DNA, 19 bp, Artificial Sequence
  Synthetic
gagaatggaagatcagggt

SEQ ID NO: 582 - RNA, 19 nt, Artificial Sequence
  Synthetic
cucuuaccuucuaguccca

SEQ ID NO: 583 - RNA, 19 nt, Artificial Sequence
  Synthetic
cauaauagcuuucccauca

SEQ ID NO: 584 - DNA, 19 bp, Artificial Sequence
  Synthetic
cataatagctttcccatca

SEQ ID NO: 585 - RNA, 19 nt, Artificial Sequence
  Synthetic
guauuaucgaaaggguagu

SEQ ID NO: 586 - RNA, 19 nt, Artificial Sequence
  Synthetic
gauaaugugacaguuggaa

SEQ ID NO: 587 - DNA, 19 bp, Artificial Sequence
  Synthetic
gataatgtgacagttggaa

SEQ ID NO: 588 - RNA, 19 nt, Artificial Sequence
  Synthetic
cuauuacacugucaaccuu

SEQ ID NO: 589 - RNA, 19 nt, Artificial Sequence
  Synthetic
gaauauggaugcauaagga

SEQ ID NO: 590 - DNA, 19 bp, Artificial Sequence
  Synthetic
gaatatggatgcataagga

SEQ ID NO: 591 - RNA, 19 nt, Artificial Sequence
  Synthetic
cuuauaccuacguauuccu

SEQ ID NO: 592 - RNA, 19 nt, Artificial Sequence
  Synthetic
caauauucuugguccuaga

SEQ ID NO: 593 - DNA, 19 bp, Artificial Sequence
  Synthetic
caatattcttggtcctaga

SEQ ID NO: 594 - RNA, 19 nt, Artificial Sequence
  Synthetic
guuauaagaaccaggaucu

SEQ ID NO: 595 - RNA, 19 nt, Artificial Sequence
  Synthetic
gagauugcuuuaaguggca

SEQ ID NO: 596 - DNA, 19 bp, Artificial Sequence
  Synthetic
gagattgctttaagtggca

SEQ ID NO: 597 - RNA, 19 nt, Artificial Sequence
  Synthetic
cucuaacgaaauucaccgu

SEQ ID NO: 598 - RNA, 19 nt, Artificial Sequence
  Synthetic
cagaagaagacauggcuca

SEQ ID NO: 599 - DNA, 19 bp, Artificial Sequence
  Synthetic
cagaagaagacatggctca

SEQ ID NO: 600 - RNA, 19 nt, Artificial Sequence
  Synthetic
gucuucuucuguaccgagu

SEQ ID NO: 601 - RNA, 19 nt, Artificial Sequence
  Synthetic
cuucuugcauuucugccua

SEQ ID NO: 602 - DNA, 19 bp, Artificial Sequence
  Synthetic
cttcttgcatttctgccta

SEQ ID NO: 603 - RNA, 19 nt, Artificial Sequence
  Synthetic
gaagaacguaaagacggau

SEQ ID NO: 604 - RNA, 19 nt, Artificial Sequence
  Synthetic
cuuuguacaaggccugcua

SEQ ID NO: 605 - DNA, 19 bp, Artificial Sequence
  Synthetic
ctttgtacaaggcctgcta

SEQ ID NO: 606 - RNA, 19 nt, Artificial Sequence
  Synthetic
gaaacauguuccggacgau

SEQ ID NO: 607 - RNA, 19 nt, Artificial Sequence
  Synthetic
gaauggaagaucaggguca

SEQ ID NO: 608 - DNA, 19 bp, Artificial Sequence
  Synthetic
gaatggaagatcagggtca

SEQ ID NO: 609 - RNA, 19 nt, Artificial Sequence
  Synthetic
cuuaccuucuagucccagu

SEQ ID NO: 610 - RNA, 19 nt, Artificial Sequence
  Synthetic
cuuaaugcguuuggaccau

SEQ ID NO: 611 - DNA, 19 bp, Artificial Sequence
  Synthetic
cttaatgcgtttggaccat

SEQ ID NO: 612 - RNA, 19 nt, Artificial Sequence
  Synthetic
gaauuacgcaaaccuggua

SEQ ID NO: 613 - RNA, 19 nt, Artificial Sequence
  Synthetic
cuaaaggauucaacuggaa

SEQ ID NO: 614 - DNA, 19 bp, Artificial Sequence
  Synthetic
ctaaaggattcaactggaa

SEQ ID NO: 615 - RNA, 19 nt, Artificial Sequence
  Synthetic
gauuuccuaaguugaccuu

SEQ ID NO: 616 - RNA, 19 nt, Artificial Sequence
  Synthetic
gauuaucuuagaaggcaca

SEQ ID NO: 617 - DNA, 19 bp, Artificial Sequence
  Synthetic
gattatcttagaaggcaca

SEQ ID NO: 618 - RNA, 19 nt, Artificial Sequence
  Synthetic
cuaauagaaucuuccgugu

SEQ ID NO: 619 - RNA, 19 nt, Artificial Sequence
  Synthetic
cauuugggcuccaaagaca

SEQ ID NO: 620 - DNA, 19 bp, Artificial Sequence
  Synthetic
catttgggctccaaagaca

SEQ ID NO: 621 - RNA, 19 nt, Artificial Sequence
  Synthetic
guaaacccgagguuucugu

SEQ ID NO: 622 - RNA, 19 nt, Artificial Sequence
  Synthetic
caaaucaccuuuauuagca

SEQ ID NO: 623 - DNA, 19 bp, Artificial Sequence
  Synthetic
caaatcacctttattagca

SEQ ID NO: 624 - RNA, 19 nt, Artificial Sequence
  Synthetic
guuuaguggaaauaaucgu

SEQ ID NO: 625 - RNA, 19 nt, Artificial Sequence
  Synthetic
caaauugaaaugugcaccu

SEQ ID NO: 626 - DNA, 19 bp, Artificial Sequence
  Synthetic
caaattgaaatgtgcacct

SEQ ID NO: 627 - RNA, 19 nt, Artificial Sequence
  Synthetic
guuuaacuuuacacgugga

SEQ ID NO: 628 - RNA, 19 nt, Artificial Sequence
  Synthetic
cuuuguggauuuagucccu

SEQ ID NO: 629 - DNA, 19 bp, Artificial Sequence
  Synthetic
ctttgtggatttagtccct

SEQ ID NO: 630 - RNA, 19 nt, Artificial Sequence
  Synthetic
gaaacaccuaaaucaggga

SEQ ID NO: 631 - RNA, 21 nt, Artificial Sequence
  Synthetic
uuaaugaaacaauaaucacuc

SEQ ID NO: 632 - RNA, 21 nt, Artificial Sequence
  Synthetic
gugauuauuguuucauuaauc

SEQ ID NO: 633 - DNA, 21 bp, Artificial Sequence
  Synthetic siRNA
gcacauauggacuaucaautt

SEQ ID NO: 634 - DNA, 21 bp, Artificial Sequence
  Synthetic siRNA
auugauaguccauaugugctg

SEQ ID NO: 635 - DNA, 6 bp, Artificial Sequence
  Synthetic
ggggcc

(19) United States
(12) Patent Application Publication
(10) Pub. No.: US 2021/0108199 A1
(43) Pub. Date: Apr. 15, 2021

(54) Treatment for aggressive cancers by targeting C9ORF72

(71) Applicant: The Trustees of Columbia University in the City of New York, New York, NY (US)

(72) Inventors: Vitalay Fomin, New York, NY (US); James Manley, New York, NY (US); Carol Prives, New York, NY (US)

(21) Appl. No.: 16/644,844

(22) PCT Filed: Sep. 6, 2018
(86) PCT No.: PCT/US18/49662

§ 371 Date: Mar. 5, 2020

Related U.S. Application Data
(60) Provisional application No. 62/554,749, filed on Sep. 6, 2017.

(51) Int. Cl. C12N 15/113; C07K 16/18; A61K 39/395; A61K 45/06; A61K 33/243; A61K 31/4745; A61K 38/21; A61P 35/00

(52) U.S. Cl. CPC C12N 15/113; C07K 16/18; A61K 39/3955; A61K 45/06; A61K 2039/505; A61K 31/4745; A61K 38/21; A61P 35/00; C07K 2317/76; A61K 33/243

(57) ABSTRACT

Knock down or other inhibition of C9ORF72 expression provides a method of treating cancer, in particular cancers susceptible to PARP inhibition such as glioblastoma. Thus, agents that target C9orf72, such as inhibitory oligonucleotides or antibodies can be used to treat cancers. A specific embodiment includes methods for treating cancers by administrating such agents, such as an inhibitory oligonucleotide that targets C9orf72. Also disclosed are methods that involve the co-administration of a PARP inhibitor and an agent that targets C9ORF72.

### CROSS-REFERENCE TO RELATED APPLICATIONS

This application claims the benefit of U.S. provisional application Ser. No. 62/554,749, filed 6 Sep. 2017. The entire contents of this application is hereby incorporated by reference as if fully set forth herein.

### GOVERNMENT FUNDING SUPPORT

This invention was made with government support under grant no. GM008798 awarded by The National Institutes of Health. The government has certain rights in the invention.

### BACKGROUND

### 1. Field of the Invention

The invention relates to the field of medicine, and in particular to oncology and treatment for cancer, in particular PARP1 sensitive cancers and cancers susceptible to methuosis. Preferred embodiments of the invention relate to treatments for glioblastoma. These methods involve inhibition or knock down of C9 expression.

### 2. Background of the Invention

Glioblastoma (GBM) is one of the most invasive and severe cancers. It almost always relapses and no effective treatments currently exist. The relapse is thought to occur because of cancer stem cells that rarely divide and thus do not take up the chemotherapeutic drugs. These cells thus survive the treatment and remain as a depot of cancer. One approach suggested to treat this type of cancer involves inducing proliferation of the glioblastoma cells, which theoretically increases the susceptibility of the previously dormant stem cells to the chemotherapeutic drugs and radiation. However, inducing higher proliferation of the cancer cells brings with it the significant risk of increasing the severity of the disease without leading to a beneficial therapeutic outcome. There is a great need in the art for effective treatments for glioblastoma.

### SUMMARY OF THE INVENTION

Therefore, embodiments of the invention include a method of treating a cancer susceptible to methuosis in a subject in need, comprising administering a therapeutically effective amount of an agent selected from the group consisting of an inhibitory oligonucleotide (IO) that reduces C9 expression, an anti-C9 antibody, and a combination thereof. In certain embodiments, the cancer is colon adenocarcinoma, esophagus adenocarcinoma, liver hepatocellular carcinoma, squamous cell carcinoma, pancreas adenocarcinoma, islet cell tumor, rectum adenocarcinoma, gastrointestinal stromal tumor, stomach adenocarcinoma, adrenal cortical carcinoma, follicular carcinoma, papillary carcinoma, breast cancer, ductal carcinoma, lobular carcinoma, melanoma, intraductal carcinoma, mucinous carcinoma, phyllodes tumor, ovarian cancer including ovarian adenocarcinoma, endometrium adenocarcinoma, granulose cell tumor, mucinous cystadenocarcinoma, cervix adenocarcinoma, vulva squamous cell carcinoma, basal cell carcinoma, prostate cancer, giant cell tumor of bone, bone osteosarcoma, larynx carcinoma, lung adenocarcinoma, kidney carcinoma, urinary bladder carcinoma, Wilm's tumor, uterine cancer, endometrial cancer and lymphoma. Preferably, the cancer is selected from the group consisting of BRCA 1- or 2-associated ovarian or breast cancer, glioblastoma, and melanoma.

In some embodiments the IO is an siRNA, an shRNA, an antisense molecule, an miRNA or a ribozyme, preferably an siRNA, for example an siRNA comprising SEQ ID NO. 631 or a biologically active fragment or variant thereof. In general, the siRNA is selected from the group consisting of SEQ ID NOs:1-634, or from the group consisting of SEQ ID NOs:8, 22, 23, 24, 26, 76, 77, 78, 112, 131, 167, 334, 335, 336, 391, 392, 393, 499, 500, 501, 631, and 633. In other embodiments, the anti-C9 antibody is a monoclonal antibody.

In other embodiments, the method further comprises co-administering a therapeutically effective amount of an agent selected from the group consisting of a PARP inhibitor, an adjunct cancer therapeutic agent, and both. The adjunct cancer therapeutic agent can comprise an antitumor alkylating agent, antitumor antimetabolite, antitumor antibiotics, plant-derived antitumor agent, antitumor platinum complex, antitumor campthotecin derivative, antitumor tyrosine kinase inhibitor, monoclonal or polyclonal antibody, interferon, biological response modifier, hormonal anti-tumor agent, anti-tumor viral agent, angiogenesis inhibitor, differentiating agent, PI3K/mTOR/AKT inhibitor, cell cycle inhibitor, apoptosis inhibitor, hsp 90 inhibitor, tubulin inhibitor, DNA repair inhibitor, anti-angiogenic agent, receptor tyrosine kinase inhibitor, topoisomerase inhibitor, taxane, agent targeting Her2, hormone antagonist, agent targeting a growth factor receptor, or a pharmaceutically acceptable salt thereof. In additional embodiments, the method further comprises treating PARP inhibition-susceptible cancer with at least one adjunct cancer therapy protocol selected from the group consisting of surgery, radiation therapy, chemotherapy, gene therapy, DNA therapy, adjuvant therapy, neoadjuvant therapy, viral therapy, RNA therapy, immunotherapy, and nanotherapy.

In some embodiments, the invention relates to an isolated siRNA molecule comprising the sequence of SEQ ID NO:631, or an isolated siRNA molecule selected from the group consisting of SEQ ID NOs:8, 22, 23, 24, 26, 76, 77, 78, 112, 131, 167, 334, 335, 336, 391, 392, 393, 499, 500, 501, 631, and 633. Additionally, the invention relates to pharmaceutical compositions comprising a pharmaceutically acceptable carrier and an siRNA that reduces C9 expression, for example wherein the siRNA is SEQ ID NO:631 or an siRNA selected from SEQ ID NOs:8, 22, 23, 24, 26, 76, 77, 78, 112, 131, 167, 334, 335, 336, 391, 392, 393, 499, 500, 501, 631, and 633. The pharmaceutical compositions optionally further comprise an agent selected from the group consisting of a PARP inhibitor, an anti-C9 antibody, and both.

### BRIEF DESCRIPTION OF THE DRAWINGS

FIG. 1A is a pair of phase contrast images (40×) of live U87 cells treated with control (siCtrl) and C9 siRNA as indicated, after 3 days.

FIG. 1B is an image of a western blot that shows that the siRNA targeting C9 reduced expression compared to control.

FIG. 2A is a graph showing vacuoles per cell for control and siC9-treated cells.

FIG. 2B is a graph showing the area of vacuoles for the same cells.

FIG. 3A is a set of phase contrast images of U2OS cells treated with the indicated siRNA.

FIG. 3B is a western blot to show C9 levels in these cells.

FIG. 3C is a graph indicating the number of vacuoles per cell in this study.

FIG. 4A is a set of immunofluorescent images showing staining of U87 cells as treated for FIG. 1A, transfected with ptfLC3 on day 2 as indicated.

FIG. 4B is a western blot indicating LAMP1 levels after control or siC9 treatment.

FIG. 5A and FIG. 5B are immunofluorescent images of U87 cells as treated for FIG. 1A, stained with MitoTracker™ for mitochondria (red) and DAPI for nuclei (blue).

FIG. 6A and FIG. 6B presents quantitation of data from FIG. 5, including mitochondrial networks (FIG. 6A) and mitochondrial count (FIG. 6B).

FIG. 7 is a set of images of U87 cells treated as indicated, in phase contrast (left), Alexa™ 488 channel (center), and a merged image (right).

FIG. 8 is a set of images (upper, fluorescent; lower, phase contrast) of U87 cells treated as indicated.

FIG. 9A, FIG. 9B, FIG. 9C, and FIG. 9D are phase contrast images of U87 cells treated as indicated.

FIG. 9E is a western blot showing the efficiency of C9 KD in EIPA-treated cells.

FIG. 10 is a graph showing vacuoles per cell in U87 cells as indicated.

FIG. 11A, FIG. 11B, FIG. 11C, and FIG. 11D are phase contrast images of U87 cells treated as indicated.

FIG. 11E is a western blot showing the efficiency of C9 KD in NSC-treated cells.

FIG. 12 is a graph showing vacuoles per cell in U87 cells as indicated.

FIG. 13A, FIG. 13B, FIG. 13C, and FIG. 13D are representative transmission electron micrographs of U87 cells treated with control or C9 siRNA.

FIG. 14 is a set of western blots of U87 cells treated with control or C9 siRNA, to show RAS, RAC1, and p-ERK levels. Actin was used as a loading control.

FIG. 15 is a graph showing normalized viability for U87 cells.

FIG. 16 is a graph presenting FACS data for U87 cells.

FIG. 17 is a western blot showing the levels of cleaved and un-cleaved caspace-3 and PARP1 as indicated, with an Actin loading control.

FIG. 18 is a graph showing the fold change in PARP1 mRNA levels as indicated.

FIG. 19A is a graph of normalized viability for the indicated treated U87 cells.

FIG. 19B is a graph of viability for the indicated treated U87 cells.

FIG. 20 is a set of phase contrast images of U87 cells treated with control or C9 siRNA and stained with β-galactosidase staining.

FIG. 21A is a graph showing fluorescence intensity (DCFDA), representing ROS levels.

FIG. 21B is a set of fluorescent images of the control and C9 KD cells.

FIG. 21C is a graph showing the results of dihydroethidium experiments.

FIG. 22 is a graph showing fluorescence intensity, representing ROS levels measured by DCFDA.

FIG. 23A presents results of a TUNEL assay of U87 cells for DNA damage.

FIG. 23B is a graph presents quantitated data from the panels of FIG. 23A.

FIG. 24A, FIG. 24B, FIG. 24C, FIG. 24D, and FIG. 24E are data as indicated for a TUNEL study for cells treated as indicated for FIG. 23.

FIG. 25A is a western blot showing γ-H2AX and ubiquitinated γ-H2AX levels in U87 cells as indicated.

FIG. 25B is a set of immunofluorescence images of U87 cells stained for γ-H2AX (green).

FIG. 25C is a graph presenting quantitated data from three replicates for γ-H2AX foci per cell.

FIG. 26 is a western blot showing phospho-p53, p53, and p21 levels with the indicated treatment with siRNA.

FIGS. 27A-27G are a set of TR-qPCR analyses of C9ORF72 (FIG. 27A), CDKN1A (p21; FIG. 27B), NOXA (FIG. 27C), PUMA (FIG. 27D), GLUT1 (FIG. 27E), GLUT3 (FIG. 27F), and MDM2 (FIG. 27G) mRNA levels.

FIG. 28 is a western blot of U2OS cells treated with control or C9 siRNA and probed as indicated on left side of the blot. Actin was a loading control.

FIG. 29A is a set of phase contrast images of U87 cells treated as indicated.

FIG. 29B is a western blot showing p53 and C9 levels under the indicated conditions.

FIG. 30A and FIG. 30B are graphs showing the vacuoles per cell and area of vacuoles under the indicated conditions, respectively.

FIG. 31 is a western blot showing p53 levels in the indicated U87 cells.

FIGS. 32A-32E are a set of TR-qPCR analyses of C9ORF72 (FIG. 32A), CDKN1A (p21; FIG. 32B), PUMA (FIG. 32C), MDM2 (FIG. 32D), and PARP1 (FIG. 32E) mRNA levels.

FIG. 33A is a set of phase contrast images of two C9 KO clones (KO #9 and KO #4) as indicated, of null U87 cells.

FIG. 33B is a western blot showing C9 levels after C9 KD in KO #4 cells.

FIG. 34 is a graph presenting quantitation of data measuring the number of vacuoles per cell.

FIG. 35 is a western blot showing PARP1, phospho-ERK, and γ-H2AX levels after C9 KD in the U87 KO #4 cell line.

FIG. 36A is a set of phase contrast images of U87 null KO #4 cells transfected with HA tagged wtp53 and treated as indicated.

FIG. 36B is a western blot showing levels of HA-p53 under the indicated conditions.

FIG. 36C and FIG. 36D are graphs presenting data on the number of vacuoles per cells and normalized viability, respectively.

FIG. 37 is a western blot of p53 null U87 cells (KO #4), treated with control or C9 siRNA and probed as indicated to the left of the blot.

FIG. 38 is a set of confocal microscopy images of two p53 null U87 clones (KO #4 and KO #9) treated with control or C9 siRNA and stained with Mitotracker™ red.

FIG. 39A and FIG. 39B are graphs showing data on fold changes in C9ORF72 levels and fold changes in EGF, respectively.

FIG. 40 is a bar graph showing cell viability of HCC cells treated with control siRNA and C9 targeted siRNA (S1 and S2).

### DETAILED DESCRIPTION

### 1. Definitions

Unless otherwise defined, all technical and scientific terms used herein are intended to have the same meaning as commonly understood in the art to which this invention pertains and at the time of its filing. Although various methods and materials similar or equivalent to those described herein can be used in the practice or testing of the present invention, suitable methods and materials are described below. However, the skilled should understand that the methods and materials used and described are examples and may not be the only ones suitable for use in the invention. Moreover, it should be understood that as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary. Hence, where appropriate to the invention and as understood by those of skill in the art, it is proper to describe the various aspects of the invention using approximate or relative terms and terms of degree commonly employed in patent applications, such as: so dimensioned, about, approximately, substantially, essentially, consisting essentially of, comprising, and effective amount.

Generally, nomenclature used in connection with, and techniques of, cell and tissue culture, molecular biology, immunology, microbiology, genetics, protein, and nucleic acid chemistry and hybridization described herein are those well-known and commonly used in the art. The methods and techniques of the present invention generally are performed according to conventional methods well known in the art and as described in various general and more specific references, unless otherwise indicated. See, e.g., Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lan, Antibodies: A Laboratory Manual, Cold Spring Harbor Laboratory Press, Cold Spring Harbor, N.Y. (1990); Principles of Neural Science, 4th ed., Eric R. Kandel, James H. Schwartz, Thomas M. Jessell editors. McGraw-Hill/Appleton & Lange: New York, N.Y. (2000). Unless defined otherwise, all technical and scientific terms used herein have the same meaning as commonly understood by one of ordinary skill in the art.

As used herein and in the appended claims, the singular forms “a,” “an,” and “the” include plural references unless specifically stated otherwise.

As used herein, the term “about” means approximately, roughly, around, or in the region of. When the term “about” is used in conjunction with a numerical range, it modifies that range by extending the boundaries above and below the numerical values set forth. In general, the term “about” is used herein to modify a numerical value above and below the stated value to refer to a range plus or minus 20 percent of the recited value, so that, for example, “about 0.125” means 0.125±0.025, and “about 1.0” means 1.0±0.2.

As used herein, an “adjunct cancer therapeutic agent” pertains to an agent that possesses selectively cytotoxic or cytostatic effects to cancer cells over normal cells. Adjunct cancer therapeutic agents may be co-administered with a C9 KD agent. In an alternative embodiment, an adjunct cancer therapeutic agent is co-administered with a C9 KD agent and a poly ADP ribose polymerase (PARP) inhibitor.

As used herein, the term “adjunct cancer therapy protocol” refers to a therapy, such as surgery, radiation therapy, chemotherapy, gene therapy, DNA therapy, adjuvant therapy, neoadjuvant therapy, RNA therapy, DNA therapy, viral therapy, immunotherapy, laser therapy, nanotherapy or a combination thereof, and may provide a beneficial effect when administered in conjunction with administration of a C9 KD agent. Such beneficial effects include reducing tumor size, slowing rate of tumor growth, inhibiting metastasis, or otherwise improving overall clinical condition, without necessarily eradicating the cancer. Cytostatic and cytotoxic agents that target the cancer cells are specifically contemplated for combination therapy. Likewise, agents that target angiogenesis or lymphangiogenesis are specifically contemplated for combination therapy.

As used herein, the terms “administering,” “administer,” and “administration,” when used with respect to an agent, means providing the agent to a subject using any of the various methods or delivery systems for administering agents or pharmaceutical compositions known to those skilled in the art.

As used herein, the term “biologically active fragment or variant thereof” refers to any siRNA or any compound that reduces the expression of C9ORF72 protein or mRNA.

As used herein, the term “C9” refers to C9ORF72 (chromosome 9 open reading frame 72) or to the gene C9orf72 in humans, which encodes this protein.

As used herein, the term “C9 KD agent” refers to an agent that reduces expression of a targeted C9ORF72 gene or protein (including by reducing transcription or translation of the gene or mRNA, respectively) and/or biological activity of C9ORF72. Examples of C9 KD agents include small molecules, polypeptides, antibodies, and C9 inhibitory oligonucleotides that reduce the expression and/or biological activity of C9ORF72.

As used herein, the term “cancer” and “tumor,” or any of their cognates, includes any neoplastic growth in a patient, including an initial tumor and any metastases. The cancer can be of the liquid or solid tumor type. Liquid tumors include tumors of hematological origin (hematological cancer), including, e.g., myelomas, leukemias, and lymphomas. Solid tumors can originate in organs, and include cancers such as lung, breast, prostate, ovary, colon, kidney, and liver. In a specific embodiment, cancer pertains to a cancer susceptible to PARP inhibition, such as glioblastoma. The terms “cancerous cell” and “cancer cell,” or any of their cognates, refer to a cell that shows aberrant cell growth, such as increased cell growth or abnormally high proliferation. A cancerous cell may be a hyperplastic cell, a cell that shows a lack of contact inhibition of growth in vitro, a tumor cell that is incapable of metastasis in vivo, or a metastatic cell that is capable of metastasis in vivo.

As used herein, the term “cancer susceptible to PARP inhibition” refers to a cancer wherein the aggressiveness, malignancy and/or viability is reduced as a result of PARP inhibition compared to non-cancer cells. This term includes glioblastoma.

As used herein, the terms “co-administration” and “co-administering” and “co-administer,” and all of their cognates refer to the administration of a first active agent before, concurrently, or after the administration of a second active agent such that the biological effects of the two (or more) agents overlap.

As used herein, the term “complementary” refers to a nucleotide sequence, usually an oligomer of at least 8-10 nucleotides, that aligns in an antiparallel manner with Watson-Crick base pairing at all positions with a target sequence for a contiguous length of nucleotides of at least 8 base pairs. Such a complementary sequence binds to the target sequence under stringent conditions. The term “substantially complementary” refers to a nucleotide sequence, usually an oligomer, that aligns in an antiparallel manner with Watson-Crick base pairing at sufficient positions with a target sequence to bind the target sequence under stringent conditions. A substantially complementary sequence is 80% homologous to a fully complementary sequence, preferably 85% c complementary, more preferably 90% complementary or 95% complementary, and most preferably 98% complementary or 99% complementary. A substantially complementary sequence can have one, two, or three non-complementary bases in the sequence, for example.

As used herein, the term “inhibitory oligonucleotide (IO)” refers to an antisense, siRNA, shRNA, ribozyme, miRNA or other oligonucleotide that reduces the expression of a targeted gene or protein (including by reducing transcription or translation of the gene or mRNA, respectively). In particular, therefore, a “C9 IO,” as used herein, refers to an antisense, siRNA, shRNA, ribozyme, miRNA or other oligonucleotide that reduces the expression of a targeted C9ORF72 gene or protein (including by reducing transcription or translation of the gene or mRNA, respectively).

As used herein, the term “isolated” as used herein with respect to nucleic acids includes any nucleic acid not in its natural form or location, such as a purified or semi-purified nucleic acid, and also includes any non-naturally-occurring synthetic nucleic acid sequence.

As used herein, the term “isolated nucleic acid” refers primarily to a gene or mRNA that is separated from other nucleic acid molecules that are present in a mammalian genome, including nucleic acids that normally flank one or both sides of the nucleic acid in a mammalian genome (e.g., nucleic acids that flank an C9 gene).

As used herein, the term “knock down (KD)” refers to a significant reduction of C9 mRNA or C9 protein (30% or more reduction). Thus, the term “C9 KD” refers to knock down of C9. The term “C9 KD agent” refers to any agent that achieves knock down of C9.

As used herein, the term “Methuosis” refers to nonaptotic cell death associated with vacuolization of macropinosome and endosome compartments.

As used herein, the term “nucleic acid” refers to both RNA and DNA, including cDNA, genomic DNA, and synthetic (e.g., chemically synthesized) DNA. The nucleic acid can be double-stranded or single-stranded (i.e., a sense or an antisense single strand).

As used herein, the term “PARP inhibitor” as used herein refers to an agent that reduces expression of a targeted poly ADP ribose polymerase (PARP) 1 or PARP 2 gene or protein, or both (including by reducing transcription or translation of the gene or the mRNA, respectively), and/or an agent that reduces the biological activity of PARP 1 or 2, that is not a C9 KD agent. The term “PARP inhibition-susceptible cancer” refers to any cancer or hyperproliferative disorder that can be reduced in severity, arrested, treated, and the like by a PARP inhibitor. Examples of such cancers include, but are not limited to glioblastoma, colon adenocarcinoma, esophagus adenocarcinoma, liver hepatocellular carcinoma, squamous cell carcinoma, pancreas adenocarcinoma, islet cell tumor, rectum adenocarcinoma, gastrointestinal stromal tumor, stomach adenocarcinoma, adrenal cortical carcinoma, follicular carcinoma, papillary carcinoma, breast cancer, ductal carcinoma, lobular carcinoma, melanoma, intraductal carcinoma, mucinous carcinoma, phyllodes tumor, ovarian cancer including ovarian adenocarcinoma, endometrium adenocarcinoma, granulose cell tumor, mucinous cystadenocarcinoma, cervix adenocarcinoma, vulva squamous cell carcinoma, basal cell carcinoma, prostate cancer, giant cell tumor of bone, bone osteosarcoma, larynx carcinoma, lung adenocarcinoma, kidney carcinoma, urinary bladder carcinoma, Wilm's tumor, uterine cancer, endometrial cancer, and lymphoma.

As used herein, the term “subject” refers to an animal being treated with one or more enumerated agents as taught herein. The term includes any animal, preferably a mammal, including, but not limited to, farm animals, zoo animals, companion animals, service animals, laboratory or experimental model animals, sport animals. More specific examples include simians, avians, felines, canines, equines, rodents, bovines, porcines, ovines, and caprines. The term specifically includes humans and human patients, particularly human cancer patients or humans in need of treatment for cancer, including glioblastoma. A suitable subject for the invention can be any animal, preferably a human, that is suspected of having, has been diagnosed as having, or is at risk of developing a disease that can be ameliorated, treated or prevented by administration of one or more enumerated agents. Therefore, a “subject in need” refers to a subject as defined herein that is suspected of having, has been diagnosed as having, or is at risk of developing a disease that can be ameliorated, treated or prevented by administration of one or more enumerated agents.

As used herein, the terms “treating” and “treatment of,” and all of their cognates, refer to providing any type of medical management to a subject. Treating therefore includes, but is not limited to, administering a composition comprising one or more active agents to a subject using any known method for purposes such as curing, reversing, alleviating, reducing the severity of, inhibiting the progression of, or reducing the likelihood of a disease, disorder, or condition or any of its symptoms, and also includes reduction or elimination of one or more symptoms or manifestations of a disease, disorder or condition.

A “therapeutically effective amount” refers to an amount which, when administered in a proper dosing regimen, is sufficient to reduce or ameliorate the severity, duration, or progression of the disorder being treated (e.g., cancer), prevent the advancement of the disorder being treated (e.g., cancer), cause the regression or remission of the disorder being treated (e.g., cancer), or enhance or improve the prophylactic or therapeutic effects(s) of another therapy. The full therapeutic effect does not necessarily occur by administration of one dose and may occur only after administration of a series of doses. Thus, a therapeutically effective amount may be administered in one or more administrations per day for successive days over a treatment regimen or course of treatment.

### 2. Overview

Overexpressing active RAS in glioblastoma cell lines leads to non-apoptotic death coined methuosis (means to drink to intoxication), which is a macropinocytosis related cell death. Inhibition or knock-down of C9orf72 (C9) causes an initial increase in proliferation for about 7 days, and then massive vacuole formation and cell death by methuosis in U87 glioblastoma (GBM) cells. Some data suggest that C9 KD leads to RAS activation which can be toxic to some cancers, for example glioblastoma. Additionally, p53 levels are elevated upon C9 KD, which further suggests that knock down of C9 may increase tumor suppressor activity. Furthermore, human bone osteosarcoma also is very sensitive to C9orf72 KD, which suggests that C9 KD can be used to treat various types of cancers.

In addition, C9 knockdown reduces PARP1 expression in GBM cells, which supports C9 knockdown therapy to treat PARP inhibition-susceptible cancers, including breast cancer, ovarian cancer and prostate cancer. Based on this discovery, one preferred embodiment of the invention pertains to a method of treating glioblastoma or PARP inhibition-susceptible cancer in a subject, comprising administering a therapeutically effective amount of an inhibitory oligonucleotide (IO) that significantly reduces C9 expression or an anti-C9 monoclonal or polyclonal antibody or a combination thereof. In a specific embodiment, the inhibitory oligonucleotide is an siRNA, shRNA, antisense molecule, miRNA or ribozyme. More specifically, embodiments of the invention include an siRNA comprising SEQ ID NO. 631, SEQ ID NO:633, or a biologically active fragment or variant thereof.

Knock down of C9orf72 causes cell death in several types of cancers, therefore, either by inhibiting PARP1 or by induction of cell death (methuosis).

### 3. Summary of Results

1. Treatment of U87 cells (GBM cell line) and USOS cells (osteosarcoma cell line) with a C9 targeting siRNA caused the formation of vacuoles in the cells.

2. Exposure of U87 cells to C9 siRNA in the presence of dextran showed that C9 KD resulted in the formation of vacuoles filled with dextran. Dextran is too big for the endocytosis pathway, thus these data suggest that vacuole formation arises from micropinocytosis.

3. C9 KD leads to RAS and p-ERK up-regulation in U87 cells and U2OS cells.

4. C9 KD increases production of reactive oxygen species (ROS) in U87 cells.

5. C9 KD increases DNA damage in cells.

6. C9 KD increases cell death in U87 cells.

7. C9 KD upregulates P53 in U87 and U2OS cells.

8. RAS upregulation caused by C9 KD in U87 cells is independent of p53.

9. C9 KD decreases PARP1 in U87 cells. This implicates C9 KD as therapy in PARP related cancers.

10. C9 KD reduces cell viability in HCC cells. This data implicates C9 KD as therapy in cancers associated with the BRCA1 gene, such as breast cancer.

### 4. Therapeutic Implications

1. C9 KD as a PARP1 inhibitor, which can be used to treat cancers with homologous recombination defects. For example, BRCA1 mutated breast cancers

2. C9 KD in glioblastoma or osteosarcoma cancers to induce methuosis

3. C9 KD as EGF inhibitor

4. Attached siRNA sequences that target C9ORF72 (in addition to the original sequence) to add as compounds that can induced methuosis and act as EGF and PARP1 inhibitors

5. C9 KD leads to both activation of p53, PARP1 and EGF inhibition all in one compound

6. Additional implication is in ALS treatment. Inhibition of macropinocytoisis in ALS patients might prevent cell death and therefore eliviate or delay ALS progression

7. Since C9 KD induced micropinocytosis, which increases intake of extracellular fluids, this can be used to increase drug delivery into cancer cells.

### 5. Embodiments of the invention

### A. General Comments

C9ORF72 (chromosome 9 open reading frame 72, referred to herein as ‘C9’) is a protein which in humans is encoded by the gene C9orf72. The human C9orf72 gene is located on the short (p) arm of chromosome 9 open reading frame 72, from base pair 27,546,542 to base pair 27,573,863. Its cytogenetic location is at 9p21.2. The protein is expressed in many regions of the brain, in the cytoplasm of neurons as well as in presynaptic terminals. Disease-causing mutations in the gene were discovered. A member of the complement membrane attack complex (MAC), C9 induces pores on cell membranes, causing lysis.

The mutations in C9orf72 are significant because it is the first pathogenic mechanism identified to be a genetic link between familial frontotemporal dementia (FTD) and of amyotrophic lateral sclerosis (ALS). As of 2012, it is the most common mutation identified that is associated with familial FTD and/or ALS. Sequencing of ALS patient genomes revealed a GGGGCC (SEQ ID NO:635) expansion on chromosome 9, in an intron of the C9orf72 gene; also present in frontotemporal dementia (FTD). The function of C9ORF72 protein is not well understood, although roles in autophagy and immune dysregulation have been suggested. To date, the involvement of C9ORF72 in cancer development has not been elucidated.

RAS overexpression in GBM cell lines leads to cytoplasmic vacuole accumulation termed methuosis, a micropinocytosis-related cell death. Macropinocytosis is an endocytotic process that mediates the uptake of non-specific extracellular components. Overexpression of mutant RasV12 resulted in cell death characterized by vacuole accumulation in GBM cells. The vacuoles lacked double membrane characteristics of autophagosomes and treatment with apoptosis inhibitors did not prevent cell death. Furthermore, overexpression of Rac1 (G12V) also lead to vacuole accumulation and non-apoptotic cell death. The vacuoles were non-autophagic (lack LC3-II), but were actually macropinosomes filled with extracellular fluid. Cell death ultimately resulted due to cells rupturing caused by the accumulation of the large cytoplasmic vacuoles.

Knock down (KD) of C9 by targeting with RNA interference molecules induces cell cycle perturbations, massive vacuole formation and cell death in GBM cells, as well as an initial increase in proliferation of the cells. The increase in proliferation, however, stops after 7 days of post-knockdown of C9, and the cells start to die. C9 depletion also led to increased ROS, DNA damage, and p53 levels along with, strongly repressed PARP1 mRNA and protein levels. The p53 CRISPR knock-out U87 cell lines did not undergo significant vacuolation upon C9 depletion, indicating that the observed methuosis is p53-dependent. C9 KD is shown herein to increase RAS levels and increases phosphorylation of the RAS target ERK, which suggests that C9 KD leads to RAS activation and inducement of macropinocytosis. In addition, human bone osteosarcoma cells are sensitive to C9 KD, suggesting that C9 KD is a viability treatment modality for various types of cancers.

PARP (poly-ADP ribose polymerase) participates in a variety of DNA-related functions including cell proliferation, differentiation, apoptosis, DNA repair and also has effects on telomere length and chromosome. Oxidative stress-induced overactivation of PARP consumes NAD+ and consequently ATP, culminating in cell dysfunction or necrosis. This cellular suicide mechanism has been implicated in the pathomechanism of cancer, stroke, myocardial ischemia, diabetes, diabetes-associated cardiovascular dysfunction, shock, traumatic central nervous system injury, arthritis, colitis, allergic encephalomyelitis, and various other forms of inflammation. PARP has also been shown to associate with and regulate the function of several transcription factors. The multiple functions of PARP make it a target for a variety of serious conditions including various types of cancer and neurodegenerative diseases.

PARP-inhibition therapy represents an effective approach to treat a variety of diseases. In cancer patients, PARP inhibition may increase the therapeutic benefits of radiation and chemotherapy. Targeting PARP inhibition may prevent tumor cells from repairing DNA themselves and developing drug resistance, which may render the cells more sensitive to existing cancer therapies. PARP inhibitors have demonstrated the ability to increase the effect of various chemotherapeutic agents (e.g. methylating agents, DNA topoisomerase inhibitors, cisplatin etc.), as well as radiation, against a broad spectrum of tumors (e.g. glioma, melanoma, lymphoma, colorectal cancer, head and neck tumors).

It has also been discovered that C9 knockdown downregulates PARP1. Accordingly, it is believed that C9 inhibition provide a new therapeutic option to treat cancers known to be susceptible to PARP inhibition.

In the context of this invention, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

Chimeric antisense compounds also come within the scope of the invention may be formed as composite structures of two or more oligonucleotides, modified oligonucleotides, or oligonucleotide mimetics as described above. Such compounds have also been referred to in the art as hybrids or gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922.

### B. C9 Inhibitory Oligonucleotides (IO)

C9 inhibitory oligonucleotides (oligonucleotides that reduce C9 expression) include any oligonucleotide that is sufficiently complementary a gene or mRNA encoding C9 so that it binds to it and significantly reduces expression of the C9 gene and/or production of the mRNA or protein, preferably the C9orf72 gene or C9ORF72 protein. Such C9 IOs include, for example, isolated small hairpin RNA (shRNA), small interfering RNA (siRNA), antisense RNA, antisense DNA, chimeric antisense DNA/RNA, microRNA, and ribozymes. A significant reduction in C9, as it pertains to this invention is a reduction of at least 30%. Therefore, “knock-down” or “KD” of C9 refers to a significant reduction of C9 mRNA or protein expression.

Certain embodiments of the present invention are directed to the use of C9 KD agents, such as antisense nucleic acids or small interfering RNA (siRNA) or short hairpin RNA (shRNA), to reduce or inhibit expression of a targeted C9 gene and hence the biological activity of the targeted C9 protein. Based on the known sequences of the targeted C9 proteins and genes encoding them, antisense DNA or RNA that are sufficiently complementary to the respective gene or mRNA to turn off or reduce expression can be readily designed and engineered, using methods known in the art. In a specific embodiment of the invention, antisense or siRNA molecules for use in the present invention are those that bind under stringent conditions to the targeted mRNA or targeted gene encoding a C9 protein as identified by the GenBank numbers NM 001256054, 203228, or to variants or fragments that are substantially homologous to the mRNA or gene encoding C9 protein. The antisense compounds of the invention are synthesized in vitro and do not include antisense compositions of biological origin.

Methods of making inhibitory oligonucleotides such as antisense nucleic acids are well known in the art. The invention comprises methods of reducing the expression of C9 protein and mRNA in cells (e.g. cancer cells) by contacting the cells, in situ or contacting isolated enriched populations of the cells or tissue explants in culture, with one or more of the antisense compounds or C9 KD agents or with any other inhibitory oligonucleotide of the invention. According to embodiments of the invention, a suitable target nucleic acid encompasses DNA encoding a C9 protein and RNA (including pre-mRNA and mRNA) transcribed from such DNA. The specific hybridization of a complementary or substantially complementary nucleic acid oligomer with its target nucleic acid interferes with the normal function of the target nucleic acid.

This modulation of function of a target nucleic acid by compounds which specifically hybridize to it is generally referred to as “antisense.” The functions of DNA to be reduced or “interfered with,” by antisense or any other inhibitory oligonucleotide, include its replication and its transcription. The functions of RNA to be reduced or “interfered with” include all vital functions such as, for example, translocation of the RNA to the site of protein translation, translation of protein from the RNA, and any catalytic activity which may be engaged in or facilitated by the RNA. The overall effect of such interference with target nucleic acid function is modulating or reducing the expression of the protein encoded by the DNA or RNA. In the context of the present invention, “modulation” means reducing or inhibiting in the expression of the gene or mRNA for a C9 protein.

In certain embodiments of the invention, targeting includes choosing a site or sites within the target DNA or RNA encoding the C9 protein for the antisense interaction to occur in order to achieve the desired inhibitory effect. Within the context of the present invention, a preferred site for interference of a gene (for hybridization of the complementary or substantially complementary sequence) is in the region encompassing the translation initiation or termination codon of the open reading frame (ORF) of the mRNA for the targeted protein. Since, as is known in the art, the translation initiation codon is typically 5′-AUG in transcribed mRNA molecules (5′-ATG in the corresponding DNA molecule), the translation initiation codon is also referred to as the “AUG codon,” the “start codon” or the “AUG start codon.”

A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine in eukaryotes. It is also known in the art that eukaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene. Routine experimentation will determine the optimal sequence of the antisense or siRNA. The terms “start codon region” and “translation initiation codon region” refer to a portion of such an mRNA or gene that encompasses from about 25 to about 50 contiguous nucleotides in either direction (i.e., 5′ or 3′) from a translation initiation codon.

It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which is known in the art to refer to the region between the translation initiation codon and the translation termination codon, is also a region which may be targeted effectively for interference or hybridization of a C9 KD agent. Other target regions include (1) the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene, and (2) the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene.

It is also known in the art that variants of mRNA can be produced through the use of alternative signals to start or stop transcription and that pre-mRNAs and mRNAs can possess more than one start codon or stop codon. Variants that originate from a pre-mRNA or mRNA that use alternative start codons are known as “alternative start variants” of that pre-mRNA or mRNA. Those transcripts that use an alternative stop codon are known as “alternative stop variants” of that pre-mRNA or mRNA. One specific type of alternative stop variant is the “polyA variant” in which the multiple transcripts produced result from the alternative selection of one of the “polyA stop signals” by the transcription machinery, thereby producing transcripts that terminate at unique polyA sites.

In a specific embodiment, a C9 inhibitory oligonucleotide is either SEQ ID NO:631 or SEQ ID NO:632 provided in Table 1, below.

TABLE 1
C9 Inhibitory Oligonucleotides.

| | Sequence | |
|---|---|---|
| Gene Name | sense (5′-3′) S1 | antisense (5′-3′) S2 |
| C9orf72- | UUAAUGAAACAAUAAUCACUC | GUGAUUAUUGUUUCAUUAAUC |
| 1/siBoth | SEQ ID NO: 631 | SEQ ID NO: 632 |

Other embodiments provided herein relate to an isolated siRNA molecule (C9 IO) comprising the sequence of SEQ ID NO:631, SEQ ID NO:632, or fragments thereof. Also provided herein is a composition that includes a combination of a C9 IO and a PARP inhibitor, and optionally a pharmaceutically acceptable carrier. Other compositions disclosed herein include a combination of a C9 IO and an anti-C9 polyclonal or monoclonal antibody.

Once one or more target sites have been identified, nucleic acids are chosen or synthesized to be complementary or substantially complementary to the target to hybridize sufficiently well and with sufficient specificity, to give the desired effect of inhibiting gene expression and transcription or mRNA translation.

While antisense nucleic acids are one form of a C9 KD agent, the present invention comprises using other oligomeric antisense compounds, including but not limited to oligonucleotide mimetics. The antisense compounds useful for C9 KD may comprise from about 8 to about 50 nucleobases (i.e., from about 8 to about 50 linked nucleosides). Typically, antisense compounds are antisense nucleic acids comprising from about 12 to about 30 nucleobases. Antisense compounds include ribozymes, external guide sequence (EGS) nucleic acids (oligozymes), and other short catalytic RNAs or catalytic nucleic acids which hybridize to the target nucleic acid and modulate its expression. Nucleic acids in the context of this invention include “oligonucleotide,” which refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

Antisense nucleic acids have been employed as therapeutic moieties in the treatment of disease states in animals and man. Antisense nucleic acid drugs, including ribozymes, have been safely and effectively administered to humans and numerous clinical trials are presently underway. Thus, nucleic acids can be useful therapeutic modalities that can be configured to be useful in treatment regimens for treatment of cells, tissues and animals, especially humans, for example to down-regulate expression of C9.

The antisense and siRNA compounds of the present invention can be utilized for diagnostics, therapeutics, and prophylaxis and as research reagents and kits. For therapeutics, an animal, preferably a human, suspected of having a disease or disorder such as cancer, which can be treated by reducing the expression of C9, is treated by administering antisense compounds in accordance with this invention. The compounds of the invention can be utilized in pharmaceutical compositions by adding an effective amount of an antisense compound to a suitable pharmaceutically acceptable diluent or carrier. The antisense compounds and methods of the invention are useful prophylactically, e.g., to prevent or delay the appearance of cancer. The antisense compounds and methods of the invention are also useful to retard the progression of cancer.

An IO of the invention can be an alpha-anomeric nucleic acid molecule. An α-anomeric nucleic acid molecule forms specific double-stranded hybrids with complementary RNA in which, contrary to the usual β-units, the strands run parallel to each other as described in Gaultier et al., Nucleic Acids. Res. 15:6625-6641, 1987. The antisense nucleic acid molecule can also comprise a 2′-o-methylribonucleotide as described in Inoue et al., Nucleic Acids Res. 15:6131-6148, or a chimeric RNA-DNA analogue as described in Inoue et al., FEBS Lett. 215:327-330, 1987. All of the methods described in the above articles regarding antisense technology are incorporated herein by reference.

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes as described in Haselhoff and Gerlach, Nature 334:585-591, 1988) can be used to catalytically cleave targeted mRNA transcripts thereby inhibiting translation. A ribozyme having specificity for a targeted-encoding nucleic acid can be designed based upon the nucleotide sequence of its cDNA. For example, a derivative of a Tetrahymena L-19 IVS RNA can be constructed in which the nucleotide sequence of the active site is complementary to the nucleotide sequence to be cleaved in the targeted mRNA. See, e.g., Cech et al., U.S. Pat. No. 4,987,071; and Cech et al., U.S. Pat. No. 5,116,742. Alternatively, a targeted C9 mRNA can be used to select a catalytic RNA having a specific ribonuclease activity from a pool of RNA molecules. See, e.g., Bartel and Szostak, Science 261:1411-1418, 1993.

Other IO that can be used to inhibit a targeted gene or mRNA such as C9 include miRNAs. For background information on the preparation of miRNA molecules, see e.g. United States Patent Application Nos. 2011/0020816, 2007/0099196; 2007/0099193; 2007/0009915; 2006/0130176; 2005/0277139; 2005/0075492; and 2004/0053411. See also U.S. Pat. Nos. 7,056,704 and 7,078,196. Synthetic miRNAs are described in Vatolin et al., J. Mol. Biol. 358:983-986, 2006 and Tsuda et al., Int. J. Oncol. 27:1299-1306. 2005. See also international Patent Application No. WO2011/127202 for further examples of interfering molecules for targeting CK-1, for example.

### C. C9 Antibodies

In some embodiments, C9 KD agents include or consist of anti-C9 antibodies. Methods of producing antibodies, including monoclonal antibodies, are well known by those of skill in the art. Therefore, those equipped with the teachings herein would be able to produce anti-C9 antibodies. Further, anti-C9 antibodies are commercially available and can be used as C9 KD agents according to this invention. Examples of commercially available antibodies include antibodies available through ThermoFisher™ (Waltham, Mass.) CAT #s PA5-31565, 702407, PA5-34936, or PA5-54905.

### D. PARP Inhibition

The enzyme poly ADP ribose polymerase (PARP) modifies nuclear proteins by poly ADP-ribosylation. PARP enzyme family members have been implicated in a number of different cancers, and knock-down of C9 can down-regulate PARP in certain cancer cells. Accordingly, by extension, cancers susceptible to PARP inhibition can be treated through C9 knockdown.

Cancers susceptible to PARP inhibition include, but are not limited to, colon adenocarcinoma, esophagus adenocarcinoma, liver hepatocellular carcinoma, squamous cell carcinoma, pancreas adenocarcinoma, islet cell tumor, rectum adenocarcinoma, gastrointestinal stromal tumor, stomach adenocarcinoma, adrenal cortical carcinoma, follicular carcinoma, papillary carcinoma, breast cancer, ductal carcinoma, lobular carcinoma, melanoma, intraductal carcinoma, mucinous carcinoma, phyllodes tumor, ovarian cancer including ovarian adenocarcinoma, endometrium adenocarcinoma, granulose cell tumor, mucinous cystadenocarcinoma, cervix adenocarcinoma, vulva squamous cell carcinoma, basal cell carcinoma, prostate cancer including prostate adenocarcinoma, giant cell tumor of bone, bone osteosarcoma, larynx carcinoma, lung adenocarcinoma, kidney carcinoma, urinary bladder carcinoma, Wilm's tumor, uterine cancer, endometrial cancer and lymphoma. In another specific embodiment the cancer susceptible to PARP inhibition comprises melanoma or glioblastoma.

In specific embodiments, cancer susceptible to PARP inhibition pertains to a cancer associated with BRCA deficiency, such as BRCA 1 or 2 associated ovarian or breast cancer. In terms of breast cancer, the breast cancer can be at stage I, II or III, or can be a metastatic breast cancer. The breast cancer generally negative for at least one of: ER, PR or HER2, and optionally is positive for at least one of ER, PR or HER2. For example, the breast cancer can be ER-negative and HER2-positive; ER-negative and both HER2-positive and PR-positive; PR-negative and ER-positive; PR-negative and HER2-positive; PR-negative and both ER-positive and HER2-positive; HER2-negative and ER-positive; HER2-negative and PR-positive; HER2-negative and both ER-positive and PR-positive; ER-negative, PR-negative and HER-2 positive; ER-negative and HER2-negative; ER-negative, HER2-negative and PR-positive; PR-negative and HER2-negative; PR-negative, HER2-negative and ER-positive; or ER-negative, PR-negative and HER2-negative. In some embodiments, the breast cancer is deficient in homologous recombination DNA repair.

In certain embodiments, cancers susceptible to PARP inhibition are treated with a therapeutically effective amount of an IO that significantly reduces expression of C9, and/or reduces PARP. In other embodiments, they are treated by administering a C9 KD agent in combination with another PARP inhibitor. Examples of some known PARP inhibitors are provided in Table 2, below. Lin et al., Cell, 169:183, 2017 and selleckchem.com/PARP are incorporated by reference for disclosures of other PARP inhibitors.

TABLE 2
Selected PARP Inhibitors.

| | Route of | |
|---|---|---|
| PARP inhibitor | administration | Histology |
| AG014699 | Intravenous | Solid tumors, Melanoma, |
| | | breast cancer |
| Veliparib (ABT 888) | Oral | Melanoma, breast cancer, |
| | | glioblastoma, ovarian cancer |
| Olaparib (AZ 2281, KU59436) | Oral | Breast cancer, ovarian |
| | | cancer, melanoma |
| Iniparib (BSI 201)/BSI 401 | Intravenous/oral | Breast cancer, non-small |
| | | cell lung cancer |
| MK4827 | Oral | Ovarian cancer |
| CEP 9722 | Oral | |
| BMN-673 | Oral | |
| Nicotinamide | Intravenous/oral | |
| INO-1001 | intravenous | Melanoma, glioblastoma |
| | | multiform |
| E7016 | Oral | |
| NMS-P118 | Oral | |
| E7449 | Oral | |
| BGP-15 | Oral/intravenous | |
| Nirparib (MK-4827) tosylate | Oral/intravenous | |
| A-966492 | | |
| PJ-34 | | |
| UPF 1069 | | |
| AZD2461 | | |
| ME0328 | | |
| NU1025 | | |
| G007-LK | | |
| NVP = TNKS656 | | |

Cancers susceptible to PARP inhibition include colon adenocarcinoma, esophagus adenocarcinoma, liver hepatocellular carcinoma, squamous cell carcinoma, pancreas adenocarcinoma, islet cell tumor, rectum adenocarcinoma, gastrointestinal stromal tumor, stomach adenocarcinoma, adrenal cortical carcinoma, follicular carcinoma, papillary carcinoma, breast cancer, ductal carcinoma, lobular carcinoma, melanoma, intraductal carcinoma, mucinous carcinoma, phyllodes tumor, ovarian cancer including ovarian adenocarcinoma, endometrium adenocarcinoma, granulose cell tumor, mucinous cystadenocarcinoma, cervix adenocarcinoma, vulva squamous cell carcinoma, basal cell carcinoma, prostate cancer, giant cell tumor of bone, bone osteosarcoma, larynx carcinoma, lung adenocarcinoma, kidney carcinoma, urinary bladder carcinoma, Wilm's tumor, uterine cancer, endometrial cancer and lymphoma. In a more particular embodiment, the cancer susceptible to PARP inhibition comprises a cancer associated with BRCA deficiency. The BRCA-deficient cancer may include BRCA 1- or 2-associated ovarian or breast cancer. Alternatively, the cancer susceptible to PARP inhibition is melanoma.

### E. Adjunct Therapies

C9 KD agents can be co-administered with an additional or adjunct cancer therapeutic agent to treat cancer. Examples of such therapeutic agents include, but are not limited to, antitumor alkylating agents, antitumor antimetabolites, antitumor antibiotics, plant-derived antitumor agents, antitumor organoplatinum compounds, antitumor campthotecin derivatives, antitumor tyrosine kinase inhibitors, monoclonal or polyclonal antibodies (e.g., antibodies directed to a tumor antigen or C9), interferon, biological response modifiers, hormonal anti-tumor agents, anti-tumor viral agents, angiogenesis inhibitors, differentiating agents, PI3K/mTOR/AKT inhibitors, cell cycle inhibitors, apoptosis inhibitors, hsp 90 inhibitors, tubulin inhibitors, DNA repair inhibitors, anti-angiogenic agents (e.g., Avastin), receptor tyrosine kinase inhibitors, topoisomerase inhibitors (e.g., irinotecan or topotecan), taxanes (paclitaxel or docetaxel), agents targeting Her2 (e.g., Herceptin), hormone antagonists (e.g., tamoxifen), agents targeting a growth factor receptor (e.g., an inhibitor of epidermal growth factor receptor (EGFR) or insulin-like growth factor 1 receptor (IGF1R), agents that exhibit anti-tumor activities, pharmaceutically acceptable salts of the above compositions, and combinations of the above compositions. In some embodiments, the adjunct cancer therapeutic agent is citabine, capecitabine, valopicitabine or gemcitabine. In some embodiments, the adjunct cancer therapeutic agent is a platinum complex. In some embodiments, the method further comprises co-administering to the patient a PARP inhibitor in combination with a C9 KD agent and at least one adjunct cancer therapeutic agent.

In some embodiments, the method further comprises administering to the patient C9 KD agent with another adjunct cancer therapy protocol. Examples of such adjunct cancer therapy protocols include surgery, radiation therapy, chemotherapy, gene therapy, DNA therapy, adjuvant therapy, neoadjuvant therapy, RNA therapy, DNA therapy, viral therapy, immunotherapy, nanotherapy or a combination thereof.

The combination of agents as taught herein can act synergistically to treat or prevent the various diseases, disorders or conditions described herein. Using this approach, one may be able to achieve therapeutic efficacy with lower dosages of each agent, thus reducing the potential for adverse side effects. Co-administration of a C9 KD agent with an adjunct cancer therapy protocol refers to administration of a C9 KD agent before, concurrently, or after conducting the adjunct cancer therapy protocol such that the beneficial effects of the co-administration overlap.

In some embodiments, the invention provides a method for inducing cellular uptake of an adjunct cancer therapeutic agent by administering a therapeutically effective amount of the adjunct cancer therapeutic agent and co-administering an amount of an IO that significantly reduces C9 expression or an anti-C9 monoclonal or polyclonal antibody or a combination thereof at an amount to increase cellular uptake of the adjunct cancer therapeutic agent. In other embodiments, the invention provides a method of treating cancer in a subject by administering a therapeutically effective amount of an IO that significantly reduces C9 gene or mRNA expression, an anti-C9 monoclonal or polyclonal antibody or a combination thereof.

### F. Pharmaceutical Compositions

Embodiments of the present invention also includes pharmaceutical compositions and formulations which include the C9 IO and C9 KD agents described herein, including but not limited to small molecules, polypeptides, antibodies, nucleic acids (including antisense RNA, siRNA, microRNAs, and ribozymes that reduce the expression and/or biological activity of C9 in cancer cells thereby causing vacuole formation. In some embodiments, the pharmaceutical compositions also include compositions and formulations of one or more PARP inhibitors and/or adjunct cancer therapeutic agents. In further embodiments, the pharmaceutical composition or formulation includes a combination of a C9 KD agent and a PARP inhibitor or adjunct cancer therapeutic agent, or both.

The therapeutic agents are generally administered in an amount sufficient to significantly reduce C9 expression thereby inducing vacuole formation in the targeted cancer cells such as glioblastoma and others listed herein, and reduce the presence of cancer cells in the subject. In a further embodiment, a first amount of a C9 IO agent or anti-C9 antibody is administered, efficacy of the first amount is determined, such as by determining the amount of C9 or C9 mRNA in serum or other tissue or by monitoring regression of tumor size via convention imaging techniques (x-ray, MRI, PET scan, CAT scan, etc.) and then administering a second amount if it is determined that the first amount was effective. The pharmaceutical compositions of the invention provide an amount of the active agent effective to treat or prevent an enumerated disease or disorder.

The pharmaceutical compositions of the present invention may be administered in a number of ways depending upon whether local or systemic treatment is desired and upon the area to be treated. Further, the C9 KD agent, PARP inhibitor, and/or adjunct cancer therapeutic agent may be combined with appropriate pharmaceutically acceptable diluent or carrier depending on the mode of administration.

In a preferred method embodiments, the compounds described herein are formulated and are administered as a pharmaceutical composition that includes a pharmaceutically acceptable carrier and one or more pharmaceutical agent, including one or more of the inventive compounds described herein, and including one or more of the inventive compounds described herein, optionally with an additional agent. A pharmaceutically acceptable carrier refers to any convenient compound or group of compounds that is not toxic and that does not destroy or significantly diminish the pharmacological activity of the therapeutic agent with which it is formulated. Such pharmaceutically acceptable carriers or vehicles encompass any of the standard pharmaceutically accepted solid, liquid, or gaseous carriers known in the art, such as those discussed in the art.

A suitable carrier depends on the route of administration contemplated for the pharmaceutical composition. Routes of administration are determined by the person of skill according to convenience, the health and condition of the subject to be treated, and the location and stage of the condition to be treated.

Such routes can be any route which the practitioner deems to be most effective or convenient using considerations such as the patient, the patient's general condition, and the specific condition to be treated. For example, routes of administration can include, but are not limited to local and parenteral, including oral, topical, transdermal, buccal, sublingual, transmucosal, wound covering, inhalation, insufflation, rectal, vaginal, nasal, wound covering, intravenous injection, intramuscular injection, intraarterial injection, intrathecal injection, subcutaneous injection, intradermal injection, intraperitoneal injection, direct local injection, and the like. The administration can be given by transfusion or infusion, and can be administered by an implant, an implanted pump, or an external pump, or any device known in the art. Preferred routes of administration include intravenous and oral. Alternatively, routine experimentation will determine other acceptable routes of administration.

Therefore, the forms which the pharmaceutical composition can take will include, but are not limited to: tablets, capsules, caplets, lozenges, dragees, pills, granules, oral solutions, powders for dilution, powders for inhalation, vapors, gases, sterile solutions or other liquids for injection or infusion, transdermal patches, buccal patches, inserts and implants, rectal suppositories, vaginal suppositories, creams, lotions, oils, ointments, topical coverings (e.g., wound coverings and bandages), suspensions, emulsions, lipid vesicles, and the like.

Any pharmaceutically acceptable carrier is contemplated for use with the invention, such as the carriers and excipients known in the art. Carriers can include, for example, starch (e.g., corn starch, potato starch, rice starch), celluloses (e.g., microcrystalline cellulose, methylcellulose, and the like), sugars (e.g., lactose, sucrose, glucose, fructose, and the like), clays, minerals (e.g., talc, and the like), gums, flavorings, odorants and fragrances, preservatives, colorings, taste-masking agents, sweeteners, gels, waxes, lipids (e.g., lipid vesicles or nanoparticles), oils, polyethylene glycols, glycerine, propylene glycol, solvents (e.g., water or pharmaceutically acceptable organic solvents), saline solutions (e.g., saline solutions, electrolyte solutions, lactated saline solutions, and the like), emulsifiers, suspending agents, wetting agents, fillers, adjuvants, dispersants, binders, pH adjusters and buffers, antibacterial agents (e.g., benzyl alcohol, methyl parabens, and the like), antioxidants (e.g., ascorbic acid, sodium bisulfite, and the like), chelating agents (e.g., EDTA and the like), glidants (e.g., colloidal silicon dioxide), and lubricants (e.g., magnesium stearate and the like). The compounds or pharmaceutical compositions containing the compounds can be provided in containers such as blister packs, ampoules, bottles, pre-filled syringes, bags for infusion, and the like. Extended and sustained release compositions also are contemplated for use with and in the inventive embodiments. Thus, suitable carriers can include any of the known ingredients to achieve a delayed release, extended release or sustained release of the active components. Preferably, the pharmaceutical compositions comprise a therapeutically effective amount.

According to an embodiment of the invention, a C9 KD agent or IO is administered to a human subject in a therapeutically effective amount by any convenient route of administration. The dose of the inventive compound is administered to the subject at convenient intervals such as every 0.5, 1, 2, 3 or more days, weekly, or at any convenient interval, in repetitive dosing regimens. The compound can be administered alone as a monotherapy, or in combination with one or more other therapies as discussed herein, either in the same dosage form or in separate dosage forms, provided at the same time or at different times, in a single dose or in a repetitive dosing regimen.

Treatment regimens include a single administration or a course of administrations lasting two or more days, including a week, two weeks, several weeks, a month, 30 days, 60 days, 90 days, several months, six months, a year, or more, including administration for the remainder of the subject's life. The regimen can include multiple doses per day, one dose per day or per week, for example, or a long infusion administration lasting for an hour, multiple hours, a full day, or longer. In some preferred embodiments, the treatment comprises a treatment cycle where treatment days and rest days of different durations are alternated. All of these treatment regimens can be developed by the practitioner of skill.

Dosage amounts per administration include any amount determined by the practitioner, and will depend on the size of the subject to be treated, the state of the health of the subject, the route of administration, the condition to be treated or prevented, and the like. In general, it is contemplated that for the majority of subjects, a dose in the range of about 0.001 mg to 5 g, about 0.05 mg to about 1 g, about 1 mg to about 750 mg, about 5 mg to about 500 mg, or about 10 mg to about 100 mg of therapeutic agent. This dose can be administered weekly, daily, or multiple times per day. A dose of about 0.001 mg, 0.01 mg, 0.1 mg, 1 mg, 5 mg, 10 mg, 50 mg, 100 mg, 500 mg, 1 g, 2 g, or 5 g can be administered. For patients weighing less than 100 kg, the recommended dosage is 0.3 mg/kg every 3 weeks by intravenous infusion. For patients weighing 100 kg or more, the recommended dosage is 30 mg (2.1), as is recommended for the first ever approved siRNA treatment, Onpattro™.

It is understood that these dose above may vary because the appropriate dose of an active agent depends upon a number of factors within the knowledge of the ordinarily skilled physician, veterinarian, or researcher. The dose(s) vary, for example, depending upon the identity, size, and condition of the subject or sample being treated, further depending upon the route by which the composition is to be administered, and the effect which the practitioner desires the an active agent to have. Appropriate doses of an active agent depend upon the potency with respect to the expression or activity to be modulated. Such appropriate doses may be determined using appropriate assays as known in the art, such as measuring the presence of C9 or C9 mRNA in a serum or tissue sample or monitoring the effect on tumor size regression based on known imaging techniques, such as xray, MRI, PET, etc. When one or more of these active agents are to be administered to an animal (e.g., a human) in order to modulate expression or activity a C9 protein, a relatively low dose may be prescribed at first, with the dose subsequently increased until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

In some embodiments of the invention, the method further comprises administering to the patient at least one PARP inhibitor, at least one anti-tumor agent, or both, along with the C9 IO of the invention. The anti-tumor agent can be one or more of any known anti-tumor agent, including but not limited to antitumor alkylating agents, antitumor antimetabolites, antitumor antibiotics, plant-derived antitumor agents, antitumor organoplatinum compounds, antitumor campthotecin derivatives, antitumor tyrosine kinase inhibitors, monoclonal antibodies, interferon, biological response modifiers, hormonal anti-tumor agents, anti-tumor viral agents, angiogenesis inhibitors, differentiating agents, or a pharmaceutically acceptable salt thereof. Particular antitumor agents include, for example, citabine, capecitabine, valopicitabine, gemcitabine, or a platinum complex. These agents are administered according to any accepted protocol known in the art. The PARP inhibitor and/or the anti-tumor agent can be administered prior to, concomitant with or subsequent to administering the C9 IO or each other.

In some embodiments, the method of treatment with a C9 IO further comprises administering to the patient a PARP inhibitor in combination with an anti-angiogenic agent such as Avastin; a topoisomerase inhibitor, such as irinotecan or topotecan; a taxane such as paclitaxel or docetaxel; an agent targeting Her-2, such as Herceptin; hormone therapy, such as a hormone antagonist tamoxifen; an agent targeting a growth factor receptor, including an inhibitor of epidermal growth factor receptor (EGFR) and an inhibitor of insulin-like growth factor 1 (IGF-1) receptor (IGF1R); or a non-pharmacological/non-biological treatment such as gamma irradiation or other radiation therapy, surgery, chemotherapy, gene therapy, DNA therapy, adjuvant therapy, neoadjuvant therapy, RNA therapy, DNA therapy, viral therapy, immunotherapy, nanotherapy or a combination thereof. These agents are administered according to any accepted protocol known in the art. The C9 IO, PARP inhibitor and/or the anti-tumor agent can be administered prior to, concomitant with or subsequent to administering any of the other agents.

In a method of treating breast cancer in a patient, a C9 IO can be administered to the patient in combination with (concomitantly, prior to or subsequent to) at least one PARP inhibitor and/or at least one anti-tumor agent. Preferably, at least one therapeutic effect, such as reduction in size of a breast tumor, reduction in metastasis, complete remission, partial remission, pathologic complete response, or stable disease, is achieved. As above, any of the agents used in combination with a C9 KD agent are administered in accordance with common practice in the art or can be determined by the practitioner using no more than routine.

### G. Methods

United States Patent Publication No. 2004/0023390, teaches that double-stranded RNA (dsRNA) can induce sequence-specific posttranscriptional gene silencing in many organisms by a process known as RNA interference (RNAi). However, in mammalian cells, dsRNA that is 30 base pairs or longer can induce sequence-nonspecific responses that trigger a shut-down of protein synthesis and even cell death through apoptosis. RNA fragments are the sequence-specific mediators of RNAi. Interference of gene expression by these small interfering RNA (siRNA) is now recognized as a naturally occurring strategy for silencing genes in C. elegans, Drosophila, plants, and in mouse embryonic stem cells, oocytes and early embryos.

In mammalian cell culture, a siRNA-mediated reduction in gene expression has been accomplished by transfecting cells with synthetic RNA nucleic. United States Patent Publication No. 2004/0023390, provides exemplary methods using a viral vector containing an expression cassette containing a pol II promoter operably-linked to a nucleic acid sequence encoding a small interfering RNA molecule (siRNA) targeted against a gene of interest.

A typical mRNA produces approximately 5,000 copies of a protein. RNAi is a process that interferes with or significantly reduces the number of protein copies made by an mRNA. For example, a double-stranded short interfering RNA (siRNA) molecule is engineered to complement and match the protein-encoding nucleotide sequence of the target mRNA the expression of which is to be reduced. Following intracellular delivery, the siRNA molecule associates with an RNA-induced silencing complex (RISC). The siRNA-associated RISC binds the target through a base-pairing interaction and degrades it. The RISC remains capable of degrading additional copies of the targeted mRNA. Other forms of RNA also can be used for this purpose, such as short hairpin RNA and longer RNA molecules. Longer molecules can cause cell death, for example by instigating apoptosis and inducing an interferon response. Cell death was the major hurdle to achieving controlled RNAi in mammals because dsRNAs longer than 30 nucleotides activated defense mechanisms that resulted in non-specific degradation of RNA transcripts and a general shutdown of the host cell. Using from about 20 to about 29 nucleotide siRNAs to mediate gene-specific suppression in mammalian cells has apparently overcome this obstacle. These siRNAs are long enough to cause gene suppression.

Certain embodiments of the invention are directed to the use of shRNA, antisense or siRNA to block expression of C9 or orthologs, analogs and variants thereof in an animal. RNA interference nucleotides can be designed using routine skill in the art to target human DNA or mRNA encoding a C9 protein as is described herein.

The invention comprises various method embodiments to deliver siRNA that is sufficiently complementary to C9 to reduce expression. There are tested delivery methods to achieve in vivo transfection such as coating siRNA with liposomes or nanoparticles. Other methods specifically target siRNA delivery to gut epithelium (Transkingdom RNA interference) using genetically engineered non-pathogenic E. coli bacteria that are able to produce short hairpin RNA (shRNA) can target a mammalian gene. Two factors were used to facilitate shRNA transfer: the invasin (Inv) and listeriolysin O (HlyA) genes. In this method, recombinant E. coli can be administered orally to deliver a shRNA against a particular targeted gene that inhibits expression of this gene in intestinal epithelial cells without demonstrable systemic complications from leaking of bacteria into the bloodstream. Certain embodiments of the invention are directed to using the Transkingdom RNA interference method adapted to siRNA that silences C9.

The inhibitory oligonucleotides of the invention are typically administered to a subject or generated in situ such that they hybridize sufficiently with or bind to cellular mRNA and/or genomic DNA encoding the protein of interest to thereby reduce expression of the protein, e.g., by reducing transcription and/or translation. The hybridization can be by conventional nucleotide complementary to form a stable duplex, or, for example, in the case of an antisense nucleic acid molecule which binds to DNA duplexes, through specific interactions in the major groove of the double helix. An example of a route of administration of inhibitory oligonucleotide molecules of the invention includes direct injection at a tissue site. Alternatively, inhibitory oligonucleotides can be modified to target selected cancer or other cells and then administered systemically. Such modifications include linking the IO to peptides or antibodies which selectively bind to target cell surface receptors or antigens. The IO can also be delivered to cells using the vectors described herein. To achieve sufficient intracellular concentrations of therapeutic IO, vector constructs in which the IO is placed under the control of a strong pol II or pol III promoter are preferred.

An increase in vacuoles/micropinocytosis in cells is an effective drug delivery enhancement system. Because increase in micropinocytosis causes increase uptake of exosomes, which can carry drugs to cells that are resistant to drug uptake.

As used herein, a therapeutically effective amount of an IO such as those hybridizing to C9 is an amount sufficient to reduce expression of a targeted C9orf72 gene or protein (including by reducing transcription or translation of the gene or mRNA, respectively), or an amount sufficient to inhibit the progression of an enumerated disease in a subject. For the purpose of the present embodiments, a significant reduction of expression involves at least a 30% reduction.

Where the inventive methods are conducted to treat a PARP inhibition susceptible cancer, embodiments of the method also can further comprise co-administering a therapeutically effective amount of a PARP inhibitor or an adjunct cancer therapeutic agent, or both. Moreover, when treating glioblastoma, in certain embodiments the method can further comprise treating the glioblastoma by administering a therapeutically effective amount of an adjunct cancer therapeutic agent.

### 6. Examples

This invention is not limited to the particular processes, compositions, or methodologies described, as these may vary. The terminology used in the description is for the purpose of describing the particular versions or embodiments only, and is not intended to limit the scope of the present invention which will be limited only by the appended claims. Although any methods and materials similar or equivalent to those described herein can be used in the practice or testing of embodiments of the present invention, the preferred methods, devices, and materials are now described. All publications mentioned herein, are incorporated by reference in their entirety; nothing herein is to be construed as an admission that the invention is not entitled to antedate such disclosure by virtue of prior invention.

### Example 1. General Methods and Materials

### A. Reagents and Plasmids

Doxorubicin hydrochloride (Sigma™, D151510MG), NUTLIN-3 (Sigma™, N6287-5MG), DMSO (Sigma™, D5879), ptfLC3 (Addgene™ plasmid #21074), PD153035 hydrochloride (Sigma™, SML0564), Dextran, Fluorescein (ThermoFisher™, D1821). HA tagged wt p53 plasmid was descried previously (Katz et al., 2018).

### B. Rat Cortical Neuron Culture

Cortices from E19 embryonic brains from pregnant Sprague-Dawley female rats were dissected in ice-cold HBSS (ThermoFisher™ Scientific #24020117), supplemented with 10 mM HEPES (pH 7.4), 10 mM MgCl₂, 1% P/S and 0.5 mM L-glutamine. The hippocampal tissue was discarded and only the cortical tissue was trypsinized for 15 minutes at 37° C. in a water bath and resuspended in DMEM with 10% FBS and 1% P/S. The dissociated neurons were subjected to centrifugation to remove debris, counted and plated onto MatTek™ 35 mm or 50 mm glass bottom dishes pre-coated with Poly-L-Lysine (1 mg/mL) and Laminin, at a density of 100,000 for live imaging experiments. After about 12-15 hours, the medium was changed to Neurobasal medium supplemented with 0.5 mM L-Glutamine, B-27 serum free supplement (Gibco™) and 1% P/S. Media were changed every 2-3 days.

### C. C9 Knockdown in Neurons and Live Imaging

Day 7 cortical neurons were transfected with 80 nM C9orf72 siRNA and 40 ng CMV-GFP vector using a magnetofectamine kit (OZ Biosciences™ #MTX0750). Seventy-two hours post-knockdown, the mitochondria were stained using 1:4000 Mitotracker™ red dye, for 3-5 minutes at 37° C., the medium was replaced with fresh Neurobasal medium and neurons expressing the GFP vector were imaged using an IX83 Andor Revolution XD™ Spinning Disk Confocal System with an environmental chamber at 37° C., a 100× oil objective (NA 1.49) and a 2× magnifier coupled to an iXon Ultra 888 EMCCD Camera at 1 fps for 5 minutes.

### D. Cell Viability Assay

For viability assays (performed in 6-well plates), cells were harvested using trypsin (0.5 ml per well) and the media were saved from each well. The saved media was re-suspended with the trypsinised cells and 2-3 aliquots (50 μL each) of this suspension were taken into a 96-well plates. CellTiter-Glo™ (CTG) Luminescent Viability assay was used to measure the viability of these aliquots. CTG-media mixture (150 μL) were added to each well. The rest of the culture was used to extract protein. The viability of cells was measured using the 1:1 dilution of the CellTiter-Glo™ Luminescent reagent (Promega™, cat #G7573) with media, which was read on Victor™ 5 plate reader after 10 minutes of shaking at room temperature. The intensity of luminescence was normalized to that of the DMSO (Sigma™, D5879) control.

### E. Galactosidase Staining

β-Galactosidase staining was performed using a Senescence™ β-Galactosidase Staining Kit (Cell Signaling™, #9860) and used according to manufacturer's instructions.

F. siRNAs

For siRNA knockdown experiments, siRNAs targeting C9 mRNA were designed with the Whitehead website (sirna.wi.mit.edu) and with RNAxs webserver (rna.tbi.univie.ac.at/cgi-bin/RNAxs). All siRNAs were obtained from GenePharma™. Lipofectamine RNAiMAX (Thermo Scientific™) was used as the transfection reagent for all siRNA experiments (according to the manufacturer's instructions). siRNA concentration was 40 nM per 60 mm plate. After 6 hours, media was changed and cells were treated again with siRNA after 48 hours. For RNA and protein analysis, cells were harvested after 48 hours following the second treatment. All KD experiments were performed in three biological replicates. The siRNA sequences were as follows: UUAAUGAAACAAUAAUCACUC (SEQ ID NO:631; sense) and GUGAUUAUUGUUUCAUUAAUC (SEQ ID NO:632; antisense); and GCACAUAUGGACUAUCAAUtt (SEQ ID NO:633; sense) and AUUGAUAGUCCAUAUGUGCtg (SEQ ID NO:634; antisense). The lower case letters in the sequence indicate location where optional modifications can be placed to enhance stability of the RNA.

G. RNA Expression, qRT-PCR

For most RNA experiments, RNA was isolated from cells using the Qiagen™ RNeasy minikit. Complementary DNA was generated using the Qiagen™ Quantitect™ reverse transcription kit with 0.5 μg of input RNA as measured with a NanoDrop spectrophotometer (Thermo Scientific™). Real-time PCR was carried out on an ABI StepOne Plus™ machine using power SYBR Green dye (Thermo Scientific™). Transcript levels were assayed in triplicate and normalized to RPL32 and HPS90 mRNAs. Relative changes in cDNA levels were calculated using the comparative Ct method (ΔΔC_{T} method). All RT-qPCR primers were designed with Primer3Plus™ or retrieved from Primerbank™. Primer sequences are listed here, including in Table 3. All primers were purchased from Life Technologies™. H. Generation of p53 Knock Out U87 Cell Lines by CRISPR

To generate p53 knockout clones, U87 cells (7×10⁵) were transfected with 2 μg p53 CRISPR/Cas9 KO plasmid (Santa Cruz Biotech™, sc-416469) using Lipofectamine 2000 (ThermoFisher™). Two days later, cells were treated with Nutlin-3 (10 μM) for 12 days to inhibit proliferation of cells with wild type p53, thereby enriching for p53 knockout cells. Single-cell clones were selected via limiting dilution, and p53 knockout clones were confirmed by western blotting using p53 antibody (FL-393).

### I. Cell Culture

U87 and U2OS cells were maintained in DMEM plus 10% fetal bovine serum (FBS) (Gemini Bio-Products™). All cells were maintained at 37° C. in 5% CO₂.

### J. Immunofluorescence.

Cells were plated on coverslips in 60-mm culture dishes, and after 48 hours washed twice with PBS followed by incubation with 4% paraformaldehyde (Sigma™) for 20 minutes. Cells then were washed three times with PBS, incubated with PBS/0.5% Triton™ X-100 for 1½ minutes, washed once with PBS and then blocked with 0.5% bovine serum albumin (Sigma™) in PBS for 30 minutes at room temperature before treatment with 100 μL of the diluted primary antibodies (1:1000) for 1 hour at room temperature. The coverslips were then washed three times with PBS and stained by incubation with 100 μL of diluted (1:100) secondary antibody (1:100). The coverslips were then washed three times with PBS and mounted on coverslips with a mounting media containing DAPI stain (Vectashield™, H-1200). Coverslips were imaged at room temperature (about 22° C.) using 40×, 1.3 NA or 63×, 1.4 NA oil immersion lenses on a confocal microscope (LSM 700; Carl Zeiss™).

### K. Dextran Measurement of Macropinocytosis.

U87 cells were treated with control and C9 siRNA for a total of 3 days. At day 2, cells were treated with a final concentration of 20 μM dextran and incubased overnight. On day 3, cells were washed with phenol red-free DMEM and then imaged using excitation/emission for fluorescein (494/521) on an LSM 700; Carl Zeiss™ microscope.

### L. Immunoblotting

For Western blot analysis, cells were lysed with lysis buffer (20 mM sodium phosphate (pH 7.0), 250 mM NaCl, 30 mM sodium pyrophosphate, 0.1% Nonidet™ P-40, 5 mM EDTA, 10 mM NaF, 0.1 mM Na₃VO₄, and 1 mM PMSF) supplemented with complete protease inhibitors (Roche™) and homogenized by passage through a 28 gauge needle. Protein concentration was measured with a Bio-Rad™ protein assay dye reagent and results were read using a spectrophotometer (BiophotometerPlus™ by Eppendorf™). Protein extracts were resolved by SDS PAGE on gradient (Thermo Fisher™) or 12% gels. Proteins were then electrotransferred at 350 mA for 80 minutes onto a PVDF membrane (Immobilon-P PVDF). Membranes were blocked with PBST with 5% milk and 1% BSA for 30 minutes. Quantitation of blots was performed Image Studio Lite software (LI-COR Biosciences™).

### M. FACS Analysis

U87 cells were treated with control and C9 siRNA for 3 days. On day 3, cells were fixed in 70% ethanol at −20° C., re-suspended in phosphate-citrate buffer (192 mM Na₂HPO₄, 4 mM citric acid), and incubated for 30 minutes in PBS containing 10 μg/mL propidium iodide (PI) and 10 μg/mL RNase A. Data were collected on a FACSCalibur™ flow cytometer (Becton Dickinson™). Data analysis was performed using FlowJo™.

### N. Antibodies

Antibodies used in the protocols include the anti-PARP1 antibody (cell signaling, 19F4), secondary anti-mouse antibody (Sigma™), p-ERK (Cell Signaling™), secondary anti-rabbit antibody (Sigma™), p-p53 (Cell Signaling™). C9 was detected using mouse monoclonal antibody from Bio-Rad™ (VMA00065). Anti-Actin (A2066), anti-Flag (F3165), mouse IgG (I5381), and rabbit IgG (I5006) antibodies were purchased from Sigma™. Anti RAS (#3965S), p-ERK (Phospho-p44/42 MAPK, #9101S), P-p53(s15, #9284S), PARP1 (#9546S), p-H2AX(S139, #2557), p21 Waf1/Cip1 (12D1, #29475), Caspase-3 (#9662S) were purchased from Cell Signaling™. Anti LAMP1 (H5G11, sc-18821), ERK (MK1, SC-135900), p53 (FL-393) were purchased from Santa Cruz™. Anti-RAC1 (05-389, 23A8) were purchased from EMD Millipore™. Anti-p53 antibodies 1801/DO1 mix.

### O. Mitochondrial Imaging

Mitchondria imaging in U87 cell was performed with MitoTracker™ Red CMXRos (ThermoFisher™, M7512) according to the manufacturer's protocol. Cells then were imaged as described herein.

### P. Mitochondria Quantitation

Quantitation was performed using a publicly available macro MiNA for ImageJ™ as described.

### Q. ROS Detection

ROS detection in U87 cells was performed using 2′,7′-dichlorofluorescin diacetate (Sigma™, D883). Cells were treated with control and siC9 siRNA. After 3 days, cells were washed with phenol red-free DMEM (ThermoFisher™, 21063029). Cells then were incubated with 20 μM DCFDA for 40 minutes at 37° C. Cells then were washed with phenol red-free DMEM. Then signal was read at Ex/Em: 485/535 nm using Synergy H1 Hybrid™ Multi-Mode Microplate reader by Biotek™. Quantitation was done after removal of background. Additionally, dihydroethidium (EMD Millipore™ 309800) was used. U87 cells were treated as described above. At day 3, cells were incubated with DHE for 30 minutes at 37° C. Cells were then washed with phenol red-free DMEM and imaged using LSM 700 (Carl Zeiss™).

### R. Macropinocytosis Inhibition

Cell were treated with control and C9 siRNA. After 6 hours, media were replaced and control and siC9 cells were either treated with DMSO or with 20 μM 5-(N-Ethyl-N-isopropyl) amiloride (Sigma™, A3085). After 3 days, cells were then imaged using Nikon Eclipse™ Ts2-FL Inverted Fluorescence Microscope and vacuoles from images were quantitated using ImageJ™.

### S. P-H2AX Foci Quantitation

Cells were treated with control and C9 siRNA for three days. Cells then were fixed and stained as described in the as described above with p-H2AX(S139, #25575) antibody. For a DNA damage positive control, 0.5 μM doxorubicin hydrochloride (Sigma™, D151510MG) was used for 24 hours before imaging. Cells were then imaged using LSM 700 Carl Zeiss™. To quantify p-H2AX foci we used a publicly available software FoCo™ as described previously.

### T. TUNEL Assay

Cells were treated with control and C9 siRNA for three days. On day three, cells were collected for TUNEL assay using the FlowTACS kit (TREVIGEN™, 4817-60-K) according to manufacturer's instructions to detect DNA fragmentation. For a positive control, DNA damage was induced using doxorubicin. Data were collected on a FACSCalibur™ flow cytometer (Becton Dickinson™) and data analysis performed on FlowJo™.

### Example 2. C9 KD Leads to Vacuole Formation and Mitochondrial Network Disorganization in U87 Cells

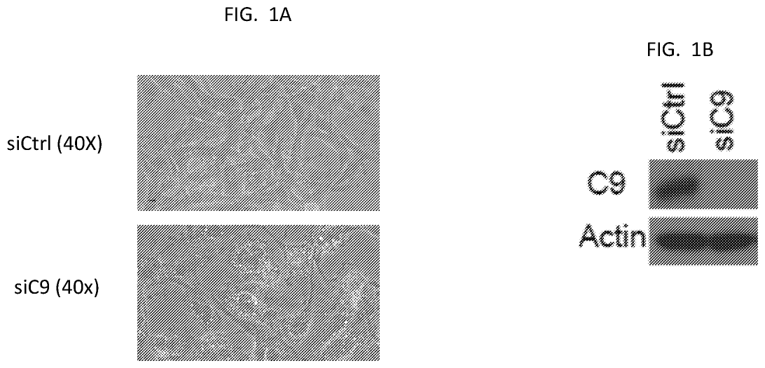

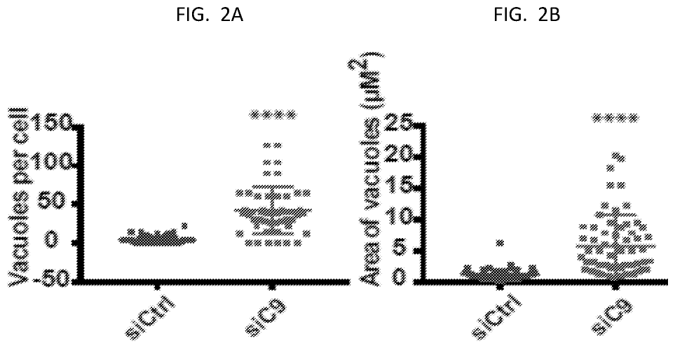

The vacuoles were examined in order to reveal the mechanism by which C9 protein levels contribute to vacuole initiation. C9 was knocked down in U87 cells. Accumulation of cytoplasmic vacuolation upon KD was observed. See FIG. 1A. Phase contrast images (40×) of live U87 cells treated with control (siCtrl) and C9 siRNA (siC9; SEQ ID NO;631) after 3 days. The scale bar represents 10 μm. FIG. 1B shows a western blot (WB) depicting knock down efficiency upon of C9 protein levels comparing control (siCtrl) and C9 siRNA (siC9) treated cells. U87 cells were treated as above and vacuoles were quantitated in two ways: the total vacuoles per cell and (see FIG. 2A) and the size of vacuoles (FIG. 2B), both of which showed significant increase upon C9 KD. The number of vacuoles per cell were counted using imageJ™ from 3 biological replicates and a total of about 50 cells were quantitated, each dot representing a measurement of vacuoles per one cell FIG. 2A). FIG. 2B shows the area/size of the vacuoles in μm². Statistical significance was assessed by two tailed t-test. Statistical significance is represented by the following: NS P>0.05, *P≤0.05, **P≤0.01, ***P≤0.001 ****P≤0.0001. Scale bar represents 10 μm.

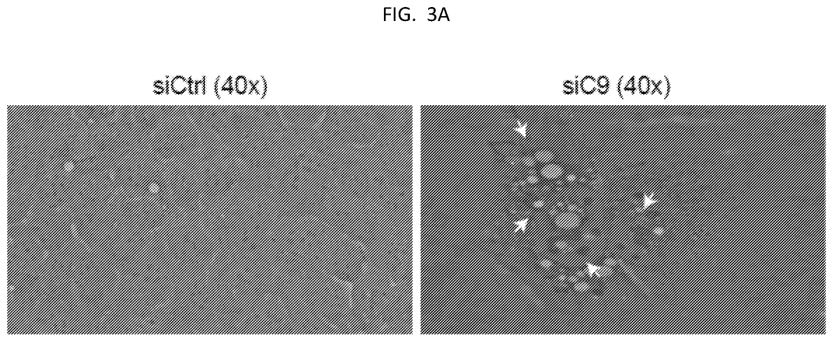

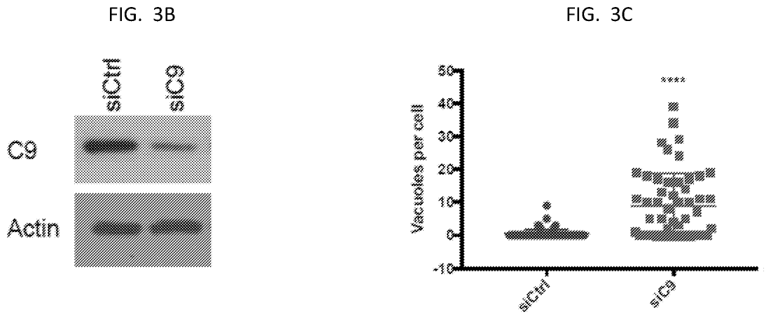

Other cell types were investigated to determine whether they also reproduce this phenotype upon C9 KD, including SH-SY-5Y, MCF7, MCF10A, HEP-G2, SK-HEP-1, HT1080 and U2OS cells). Only the osteosarcoma U2OS cell line reproduced vacuolation upon C9 KD, suggesting that this phenotype is not unique to U87 or glioblastoma cell lines. See FIG. 3A, which shoes phase contrast images (40×) of U2OS cells treated with control and C9 siRNA. The white arrows indicate the location of vacuoles. FIG. 3B presents a WB probed for C9 levels and actin as loading control. FIG. 3C is a graph showing quantitation of the amount of vacuoles per cells in control and c9 KD cells, as indicated in the figure. The statistical significance was assessed by unpaired t-test: NS P>0.05, *P≤0.05, **P≤0.01, ***P≤0.001 ****P≤0.0001.

### Example 3. Vacuole Origin Studies

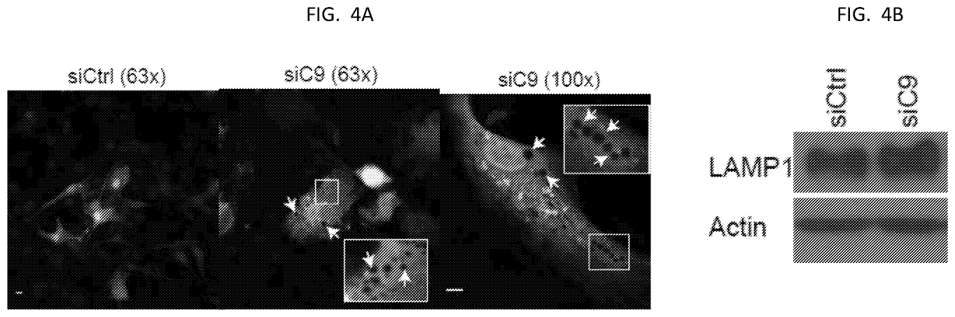

The origin of the vacuoles were characterized to determine if they were autophagic because several reports indicated that C9ORF72 is involved in membrane trafficking and autophagy in human cell culture models and mice. To this end, C9 was knocked down in U87 cells and employed a fluorescent autophagy marker ptfLC3 to mark autophagosomes and autophagolysosomes. Surprisingly, the fluorescent images revealed that vacuoles resulting from C9 KD were not stained by LC3, suggesting that these vacuoles are not autophagic. See FIG. 4A and FIG. 4B. However, an increase fluorescence of autophagolysosomes was observed, which is also supported by increased levels of lysosomal marker LAMP1 in C9 KD samples. FIG. 4A shows immunofluorescent (IF) staining of U87 cells treated as above, but transfected at day two with ptfLC3. The left panel shows control cells transfected with ptfLC3 plasmid and nuclei stained with DAPI (blue). The middle panel shows C9 siRNA-treated cells transfected with ptfLC3 and nuclei stained with DAPI (blue). The white arrows show vacuoles and the white box represents a zoom in of the cell area with vacuoles. The right IF image shows C9 siRNA-treated cells transfected with ptfLC3 with white arrows showing vacuoles. The white box here represents a zoom in of the cell area with vacuoles, nuclei stained with DAPI (blue). FIG. 4B is a WB showing LAMP1 levels after control (siCtrl) and C9 KD (siC9; SEQ ID NO:631).

### Example 4. Vacuoles are not of Mitochondrial or Autophagic Origin

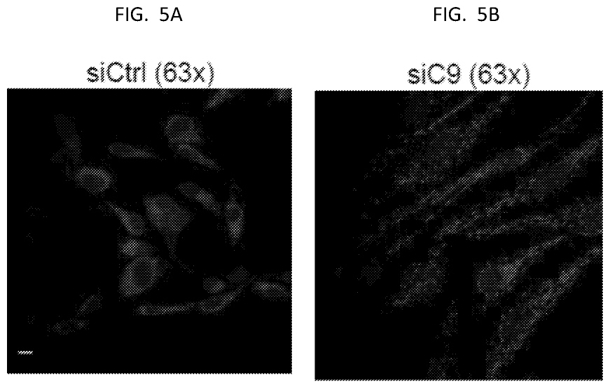

The vacuoles were studied to investigate whether they are swollen mitochondria, because several reports observed vacuolated mitochondria in ALS patients and mice models that resemble the phenotype seen here. A fluorescent mitochondrial dye was used to visualize mitochondria upon C9 KD. This mitochondrial staining revealed that the vacuoles are not swollen mitochondria, since the vacuoles did not stain red. C9 KD did cause significant mitochondrial network disorganization, however, when compared to control. See FIG. 5A and FIG. 5B, which show an IF image of U87 cells treated as in Example 1 and stained with MitoTracker (red) and nuclei stained with DAPI (blue).

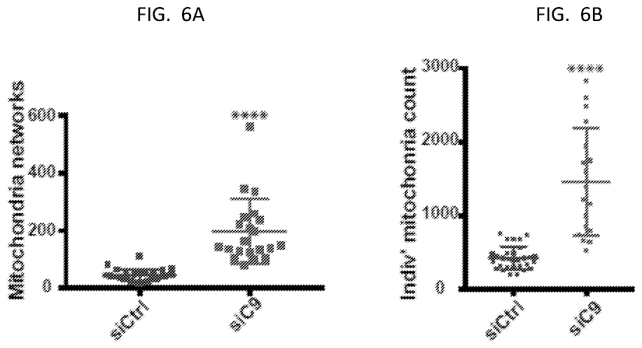

The total amount of individual and networked mitochondria staining also increased. See FIG. 5 and FIG. 6. To obtain the data seen in FIG. 6, the stained mitochondria from FIG. 5 were IF stained and analyzed using MiNA software for ImageJ™. FIG. 6A shows a quantitation measuring the amount of mitochondrial networks per cell in about 39 cells. FIG. 6B shows data for the number of individual mitochondria per cell in about 30 cells.

Mitochondrial accumulation then was investigated in other cell lines. C9 KD was performed in HeLa cells, followed by a fluorescent mitochondrial dye for live imaging. Upon C9 KD, HeLa cells showed mitochondrial network disorganization compared to control. To study the mitochondrial dysfunction phenotype upon C9 KD in a non-cancer cell line, C9 was knocked down in rat-derived cortical neurons in combination with a mitochondrial stain. Live imaging of mitochondria in cortical neurons revealed that C9 KD induced mitochondria accumulation in the dendrites. Additionally, mitochondria motility was significantly reduced in neurons with C9 KD. These results indicated that lower C9 levels can lead to mitochondrial dysfunction, but excludes the vacuoles being of mitochondrial or autophagic origin.

### Example 5. Macropinocytotic Origin for Vacuoles

In order to determine whether the vacuoles are of endocytotic origin (i.e., whether the vacuoles originate from a clathrin-independent endocytotic pathway, macropinocytosis. Macropinocytosis is a non-specific pathway by which cells are able to internalize large quantities of extracellular material and is dependent on actin membrane ruffling. This was studied because this phenomenon is characterized by large vacuoles (more than 2 μm in diameter), which is consistent with the observations here in U87 and U2OS cells.

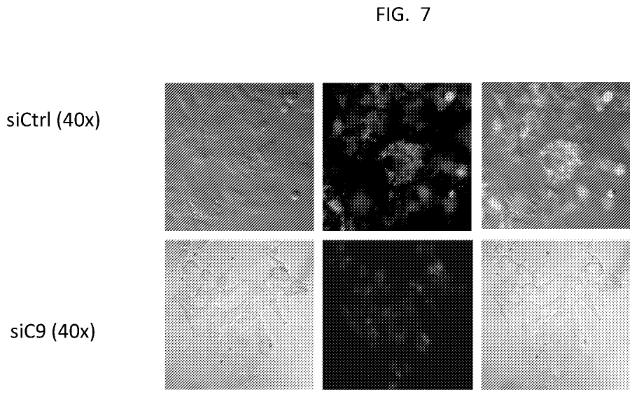

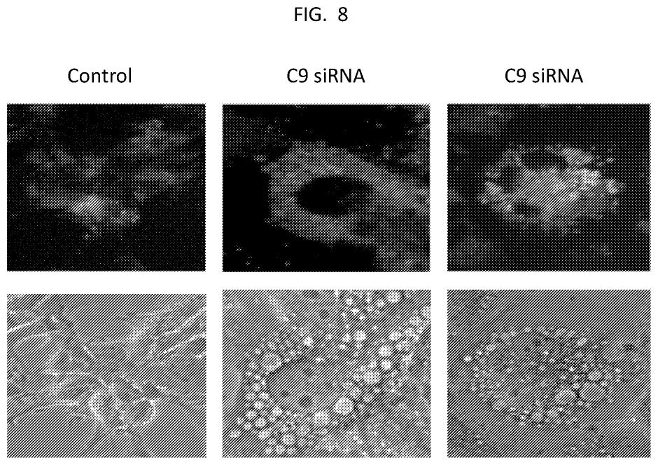

In this study, a fluid phase marker, fluorescein dextran, was added to the media. If the vacuoles are macropinosomes, the marker will be taken up and internalized, resulting in green fluorescent macropinocytosis. After dextran addition to control and C9 KD U87 cells, imaging revealed that the cytoplasmic vacuoles were stained with dextran and were green, which suggests they originate from macropinocytosis. See FIG. 7 and FIG. 8.

FIG. 7 shows U87 cells treated with Control (siCtrl) and C9 (siC9; SEQ ID NO:631) siRNA, subjected to fluorescein dextran and imaged with phase contrast (left (40×)) and with Alexa™ 488 channel (middle). The right images are a merge between the left and middle images. The scale bar represents 10 μm. White arrows indicate the position of vacuoles. In FIG. 8, the upper panels are fluorescent images (Alexa™ 488) of U87 cells treated with control (siCtrl) and C9 (siC9; SEQ ID NO:631) siRNA as indicated incubated in fluorescein dextran (green). The lower panels are the same cells imaged in phase contrast.

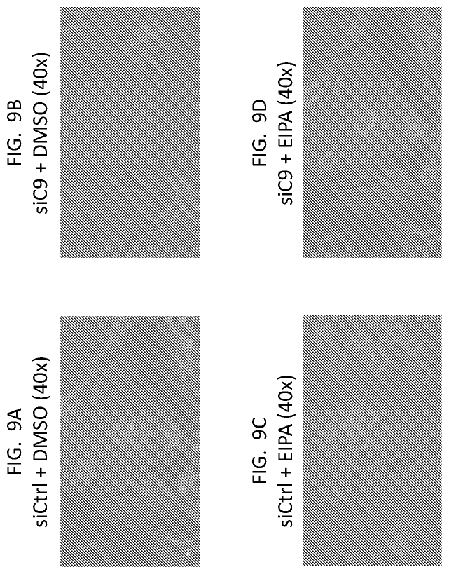

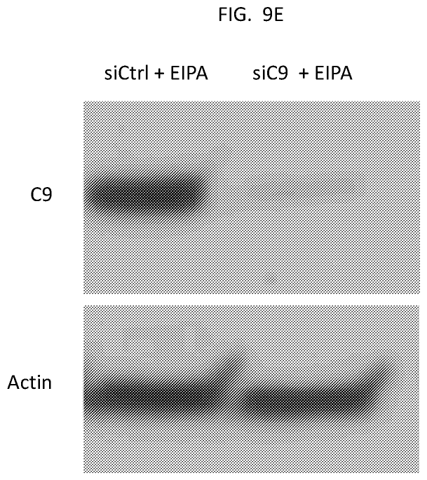

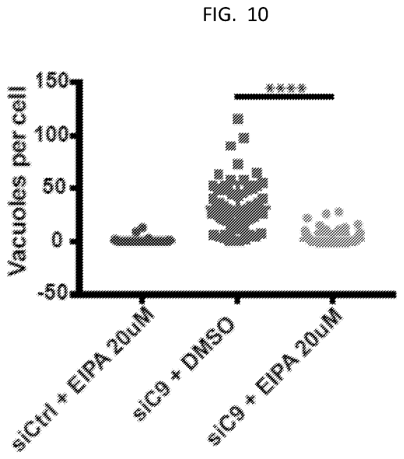

In another study to further confirm the macropinocytotic origin of the vacuoles, 5-(N-ethyl-N-isopropyl) amiloride (EIPA), a macropinocytosis inhibitor was used. C9 KD cells treated with EIPA showed significant inhibition of vacuole formation when compared to the control C9 KD DMSO-treated cells, which developed extensive cytoplasmic vacuoles. See FIG. 9 and FIG. 10.

FIG. 9 presents phase contrast images (40×) of U87 cells treated with control or C9 siRNA (SEQ ID NO;631) and incubated in DMSO (siCtrl+DMSO and siC9+DMSO, respectively) or in 20 μM Amiloride (siCtrl+EIPA 20 μM and siC9+EIPA 20 μM, respectively). The scale bar represents 10 μm. WB on the right represents C9 KD efficiency in EIPA treated U87 cells. FIG. 10 shows quantitation of the number of vacuoles per cell in about 80 U87 cells treated with control siRNA and 20 μM EIPA (blue dots), C9 treated siRNA with DMSO (red squares) and C9 treated siRNA with 20 μM EIPA (green dots). Statistical significance was assessed by an unpaired t-test: NS P>0.05, *P≤0.05, **P≤0.01, ***P≤0.001 ****P≤0.0001.

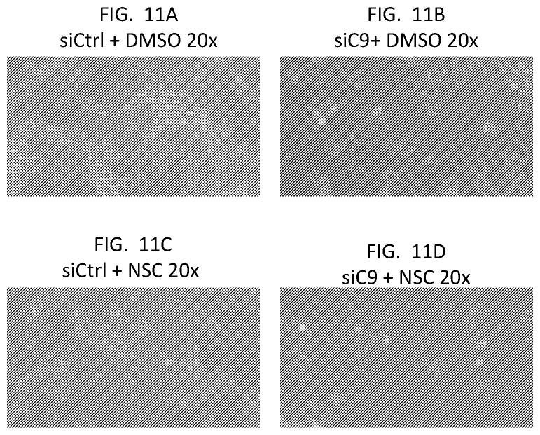

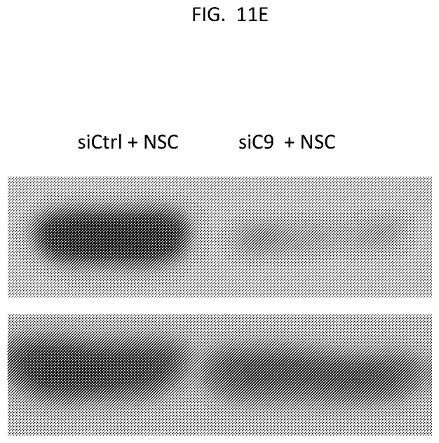

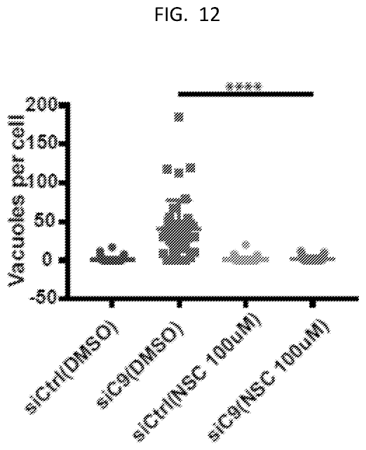

Macropinocytosis is known to depends on RAC1 activation which induces membrane ruffling (through actin rearrangement) that mature into macropinosomes. If the accumulated vacuoles are of macropinocytotic origin, then inhibition of RAC1 should rescue their accumulation. To test this, cells were treated with control or C9 siRNA in combination with NSC23766 (NSC), a RAC1 inhibitor. After 3 days of siRNA KD, vacuoles were observed in the DMSO treated cells, but cells treated with NSC were rescued from vacuole accumulation (see FIG. 11). Quantitation of the vacuoles per cells confirmed the observation and showed a significant drop in vacuoles upon NSC treatment in C9 KD cells (see FIG. 12).

FIG. 11A, FIG. 11B, FIG. 11C and FIG. 11D are a set of phase contrast images (20×) of U87 cells treated with control or C9 siRNA (SEQ ID NO:631) and incubated in DMSO (siCtrl+DMSO and siC9+DMSO, respectively) or in 100 μM RAC1 inhibitor NSC23766 (siCtrl+NSC and siC9+NSC, respectively). The scale bar represents 10 μm. The WB in FIG. 11D shows C9 KD efficiency in NSC treated U87 cells. FIG. 12 is a graph representing quantitation of the data for U87 cells treated with control (n=76) siRNA and DMSO (blue dots), U87 cells treated with C9 siRNA (n=41) with DMSO (red squares), U87 cells treated with control (n=74) siRNA and 100 μm of NSC (green dots), and U87 cells treated with C9 siRNA (n=73) with 100 μm of NSC (purple dots). Statistical significance was assessed by an unpaired t-test: NS P>0.05, *P≤0.05, **P≤0.01, ***P≤0.001 ****P≤0.0001.

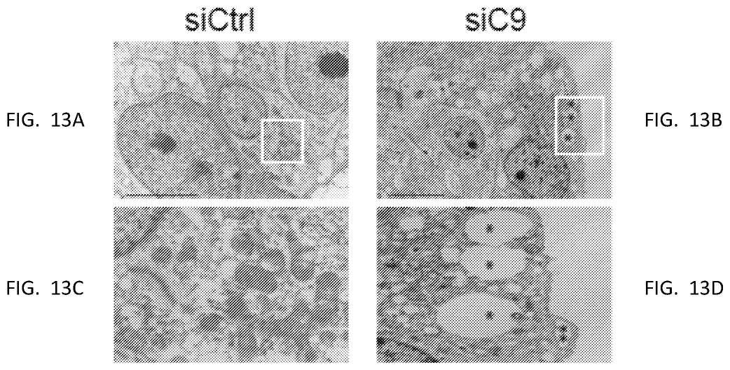

Since endocytic vacuoles (including macropinosomes) are known to have a single membrane U87 cells treated with control and siC9 siRNA (SEQ ID NO:631) were imaged using transmission electron microscopy (TEM). TEM images revealed that the vacuoles generated in C9 KD cells were electron-transparent and had a single membrane (see FIG. 13), consistent with the vacuoles being of macropinocsytotic origin. FIG. 13 is a set of representative TEM images of U87 cells treated with Control (siCtrl) and C9 (siC9) siRNA after 72 hours of KD. One asterisk indicates representative vacuoles; two asterisks represent membrane folding. The white boxes in FIG. 13A and FIG. 13B show locations for the digital zoomed-in images below, in FIG. 13C and FIG. 13D. The scale bar represents 5 μm.

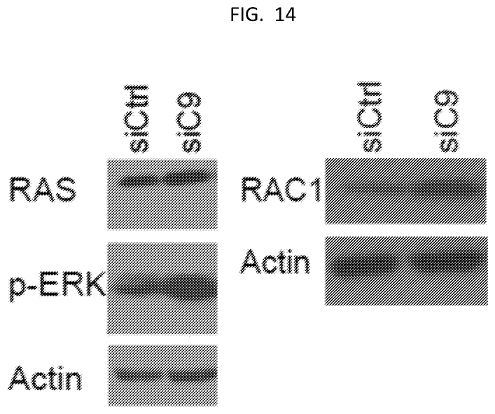

Macropinocytosis is reported to be dependent on RAS and/or RAC1 activation, therefore whether the RAS/RAC1 signaling pathway is activated upon C9 KD was explored. First, RAS and RAC1 protein levels were measured in U87 cells upon C9 KD. The data in FIG. 14 shows that they are elevated. Since levels of RAS or RAC1 are not indicative of whether they are active (GTP bound) or inactive (GDP bound), a downstream target of RAS/RAC1, ERK1/2, was measured, since both RAS and RAC1 are known to phosphorylate ERK1/2 when in their active form (GTP bound). If the RAS/RAC1 pathway is active, then ERK1/2 phosphorylation should be observed. FIG. 14 is a WB of U87 cells treated with control (siCtrl) and C9 siRNA (siC9; SEQ ID NO:631) measuring protein levels of RAS and phospho-ERK and loading control actin (left) and a WB showing RAC1 levels under the same conditions with actin as loading control (right). The western blot analysis revealed that C9 KD lead to increased phosphorylation of ERK1/2 (FIG. 14), which is consistent with increased macropinocytosis and activated RAS/RAC1 signaling. Therefore both macropinocytosis and RAS/RAC1 signaling pathways are a possible therapeutic targets in C9 ALS.

### Example 6. C9 KD leads to Non-Apoptotic Cell Death

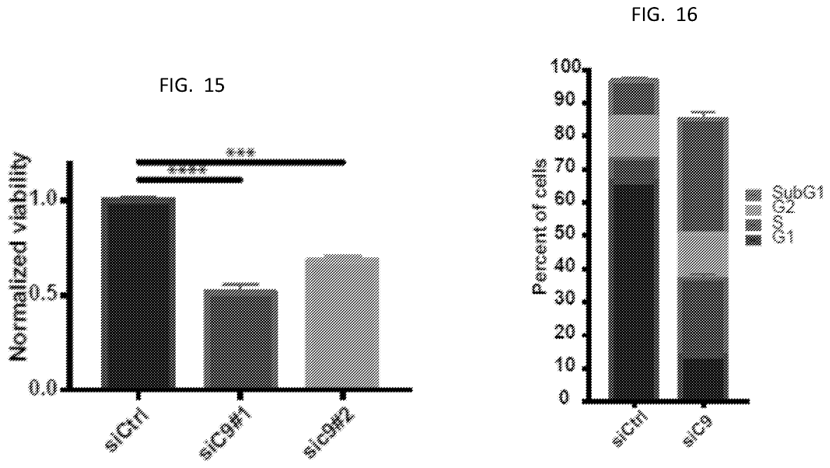

Whether the large cytoplasmic vacuoles affected cell viability was investigated next. C9 was knocked down with two independent siRNAs they were examined for viability. This test revealed that there is about a 2-fold decrease in cell viability upon treatment with two independent C9 siRNA's (see FIG. 15).

FIG. 15 is a bar graph presenting data for the normalized cell viability assay comparing control (siCtrl) and two independent C9 siRNAs (siC9 #1 (SEQ ID NO:631), siC9 #2(SEQ ID NO:633)). The statistical significance was assessed by an unpaired t-test: NS P>0.05, *P≤0.05, **P≤0.01, ***P≤0.001 ****P≤0.0001. Error bars represent standard error of the mean (SEM) from three independent biological replicates.

To validate the cell viability assays, C9 was knocked down in U87 cells and the cells were analyzed by FACS. The FACS analysis revealed a 3.1-fold increase in SubG1 phase in C9 KD cells (see FIG. 16). SubG1 phase measures apoptotic cells with fragmented DNA, indicating that C9 KD leads to cells death. Additionally, FIG. 16 shows a decrease in G1 (4.7 fold) and an increase in S phase (3.3 fold), suggesting that C9 levels affect cell cycle. FIG. 16 shows cells treated as described for FIG. 15, and on day three analyzed by FACS. The bar plots represent the cell cycle measurement (subG1-purple, G2-green, S-red, G1-blue) of three independent biological replicates upon control (siCtrl) or C9 (siC9; SEQ ID NO:631) siRNA. Error bars represent S.E.M.

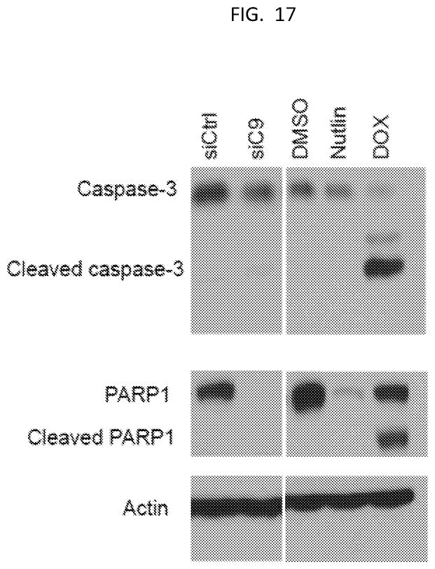

To check known markers of apoptosis, including caspase-3 cleavage and PARP1 cleavage, U87 cells were treated with doxorubicin as a positive control for apoptotic markers. C9 KD did not lead to caspase 3 cleavage (see FIG. 17). Additionally, not only was PARP1 cleavage not observed, there was no PARP1 protein observed (wee FIG. 17). FIG. 17 is a set of WB visualizing the levels of cleaved and un-cleaved caspace-3, and cleaved and un-cleaved PARP1 in cells treated with control (siCtrl) and C9 (siC9; SEQ ID NO:631) siRNA, and U87 cells treated.

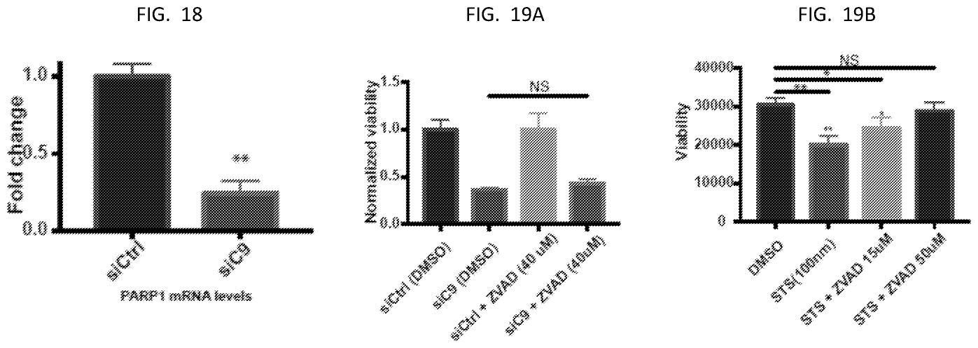

To check if PARP1 downregulation occurred on the mRNA level as well, qRT-PCR results revealed that C9 KD led to significant repression of PARP1 mRNA (see FIG. 18). FIG. 16 shows U87 cells treated as described for FIG. 15 and then mRNA levels of PARP1 were measured by qRT-PCR (n=3). Statistical significance was assessed by an unpaired t-test: NS P>0.05, *P≤0.05, **P≤0.01, ***P≤0.001 ****P≤0.0001. Error bars represent S.E.M. The lack of caspase-3 and PARP1 cleavage suggested that U87 cells undergo a non-apoptotic death.

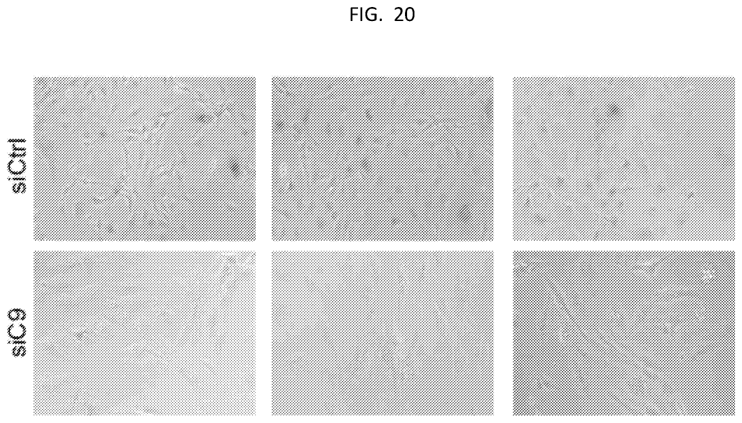

To confirm that cells undergo non-apoptotic death, apoptosis was blocked with a broad caspase inhibitor Z-VAD-FMK upon C9 KD. The cell viability assay indicated that treatment with Z-VAD-FMK did not significantly rescue C9 KD U87 cells (see FIG. 19). Additionally, the apoptosis inducer, staurosporine (STS), was used to asses Z-VAD-FMK efficacy. Z-VAD-FMK was able rescue viability of STS U87 treated cells (FIG. 19). Since Z-VAD-FMK was not able to significantly increase cell viability upon C9 KD, this suggests that the cell death is non-apoptotic. Additionally, C9 KD leads to an increase in both nucleus and cell size, which is a phenotype consistent with senescent cells. A beta galactosidase staining technique was used to asses senescence upon C9 KD. Image analysis revealed that although cells increased in size, they did not stain with the assay, which suggests that senescence is not involved in the decrease of cell viability upon C9 KD. See FIG. 20, for which U87 cells were treated with control (siCtrl) and C9 (siC9; SEQ ID NO:631) siRNA and then subjected to β-Galactosidase Staining.

### Example 7. C9 KD Leads to Methuosis-Like Cell Death and Increased ROS and DNA Damage

The morphological changes upon C9 KD resemble the non-apoptotic cell death methuosis. Methuosis is a caspase-independent cell death characterized by substantial accumulation of large non-autophagic cytoplasmic vacuoles. Methuosis has been observed in nematodes and in human cultured cancer cell line. Overexpression of constitutively active forms of RAS or RAC1 in glioblastoma cell lines has led to a catastrophic vacuolization and cell death that could not be rescued with apoptosis inhibitors. Interestingly, looking at the mutational landscape of glioblastoma cells, it is very rare to see any RAS (NRAS, KRAS, HRAS) mutations. The most abundant cancer with RAS mutations is pancreatic cancer, followed by myeloma and colorectal cancers. On the other hand, cancers that rarely exhibit RAS mutations are uveal melanoma, small cell lung carcinoma and glioblastoma (GBM). RAS mutations account for only about 3 percent of all GBM cases. The low frequency of RAS mutations in GBM combined with reports that oncogenic RAS/RAC1 mutations induce methuosis in GBM suggests that RAS mutations are of low frequency because they are lethal in GBMs. This is consistent with the observations here since C9 KD leads to RAS/RAC1 signaling pathway activation and induction of methuosis-like cell death in U87 and U2OS cell lines. RAS/RAC1 activation has been reported to increase reactive oxygen species (ROS) by activation of the NADPH oxidases NOX1/2. Additionally, we observed defects in mitochondrial organization and mitochondrial accumulation here. In addition to NADPH oxidases, mitochondrial oxidative phosphorylation can generate ROS, and accumulation of dysfunctional mitochondria is also reported to increased ROS production.

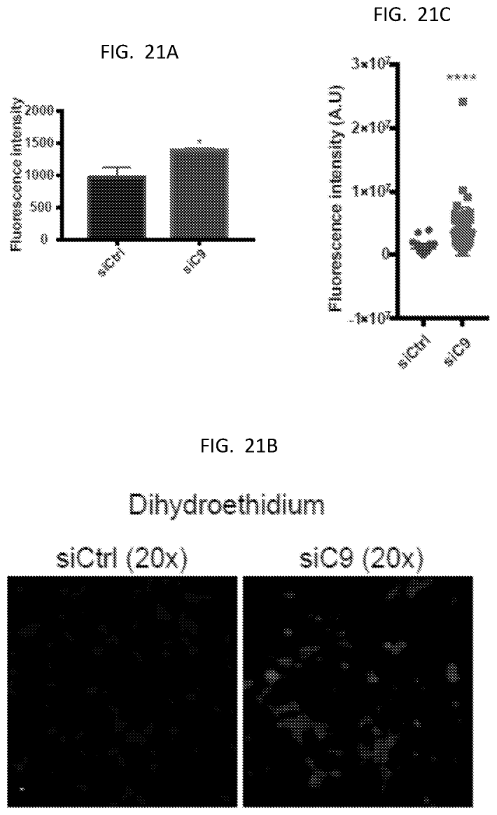

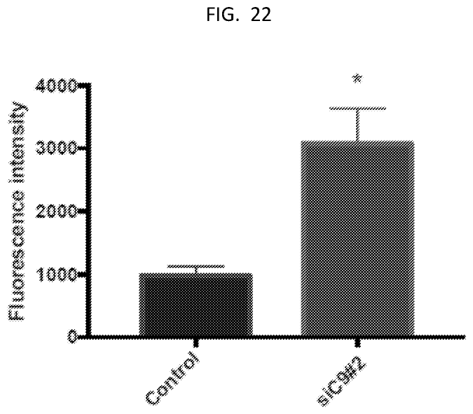

For the above reasons, whether C9 KD may lead to increase ROS production was investigated. A fluorogenic dye DCFDA, which measures ROS levels, was used. C9 was knocked down in U87 cells with two independent siRNAs and the ROS levels were measured after KD. Analysis of the ROS levels revealed that C9 KD lead to increased ROS levels with both siRNAs. See FIG. 21 and FIG. 22. Since ROS measurements are known to be sometimes unreliable, increased ROS upon C9 KD was confirmed with another independent method. Therefore, the superoxide indicator dihydroethidium (DHE) was used. DHE ROS measurements confirmed the DCFDA results and showed a significant upregulation of ROS upon C9 KD. See FIG. 21.

For the data presented in FIG. 21, U87 cells were treated with control (siCtrl) and C9 (siC9; SEQ ID NO:631) siRNA and on day three ROS levels measured by DCFDA and dihydroethidium. FIG. 21A presents data for fluorescent intensity between control (siCtrl) and C9 KD (siC9) cells as measured by DCFDA. FIG. 21B shows fluorescent images of control (siCtrl) and C9 KD (siC9) cells. FIG. 21C presents fluorescent intensity quantitation for about 30 cells from the dihydroethidium experiments (n=3). Statistical significance was assessed by an unpaired t-test: NS P>0.05, *P≤0.05, **P≤0.01, ***P≤0.001 ****P≤0.0001. Error bars represent S.E.M. Scale bar represents 10 μm. FIG. 35 shows data for cells treated as described for FIG. 21 (with the exception of using a different C9 siRNA (SEQ ID NO:631)) and ROS levels measured by DCFDA.

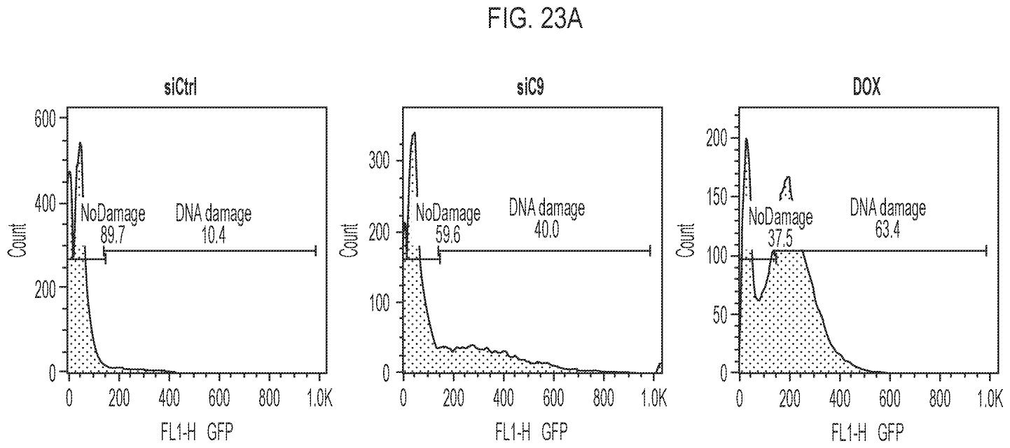

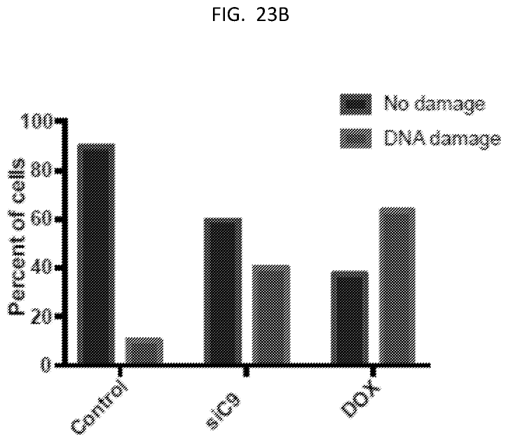

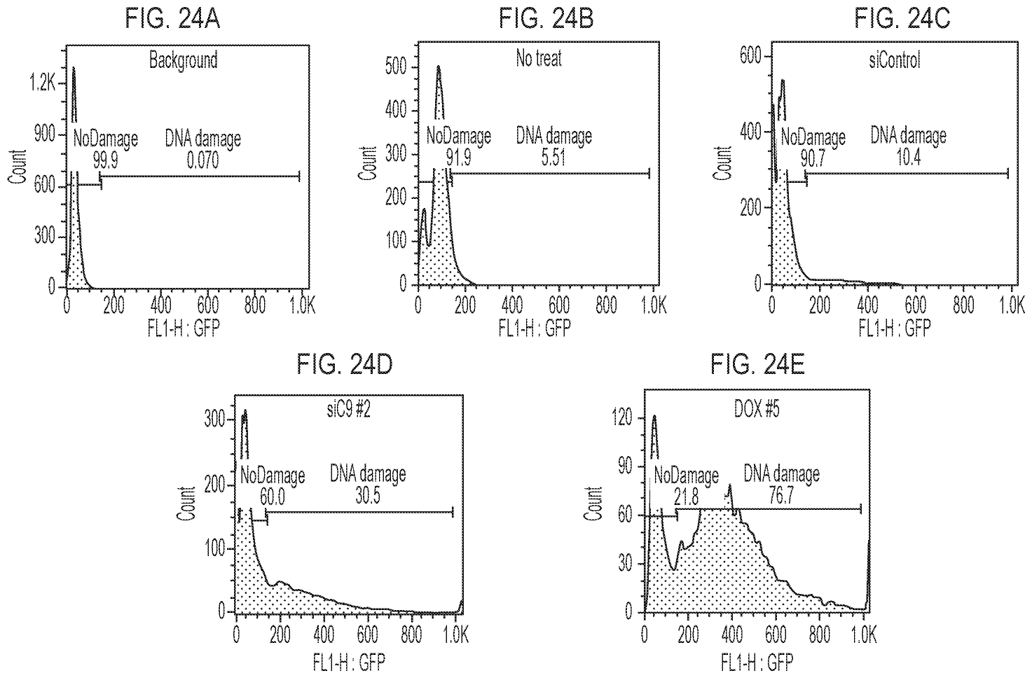

ROS can cause oxidative DNA damage. Since elevated ROS levels were observed under C9 KD conditions, it may cause increase in DNA damage. To test this hypothesis, a TUNEL assay was used to measure DNA breaks. The TUNEL assay indicated that there is an increase in DNA breaks upon C9 KD levels compared to control (see FIG. 23 and FIG. 24). In addition to C9KD, U87 cells were treated with a known chemotherapeutic DNA damage-inducing drug to serve as a positive control for DNA damage (see FIG. 23). FIG. 23 presents data for the TUNEL assay of U87 cells treated as described for FIG. 21 and for positive DNA-damage control with 0.5 μM doxorubicin (DOX). FIG. 23A presents data for the percentage of cells with DNA fragmentation (shown by DNA damage) comparing control (siCtrl) C9 KD (siC9; SEQ ID NO:631) and DOX-treated cells as measured by FACS analysis. FIG. 23B shows the quantitation of the three panels. FIG. 24 is a set of graphs showing data for cells treated as described for FIG. 23, with the exception of using a different C9 siRNA, indicated as siC9 #2 (SEQ ID NO:633).

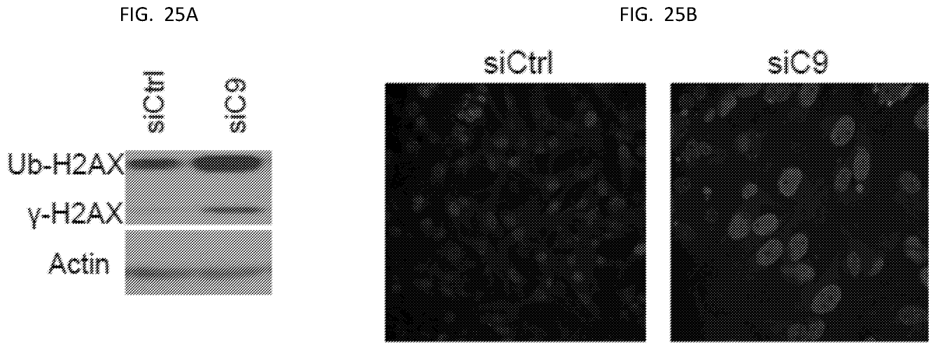

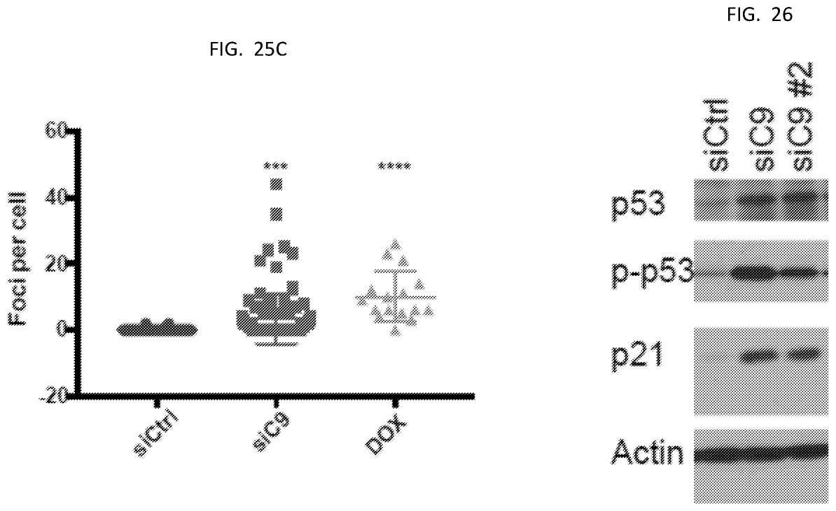

To further confirm the presence of DNA damage upon C9 KD, the DNA damage marker γ-H2AX was used as a probe on C9 KD. This WB analysis revealed that C9 KD lead to increased γ-H2AX levels (FIG. 25), suggesting an increase in DNA damage. Immunofluorescence was used to image and quantitate γ-H2AX foci in control, C9 KD and doxorubicin-treated U87 cells. Quantitation of γ-H2AX foci confirmed our previous TUNEL and WB analysis and showed a significant increase in γ-H2AX foci upon C9 KD. These results taken together confirm the presence of increased DNA damage upon C9 KD.

FIG. 25A is a WB showing γ-H2AX and ubiquitinated γ-H2AX, control (siCtrl) and C9 KD (siC9; SEQ ID NO:631), with loading of control actin. FIG. 25B is a set of IF images of U87 cells stained for γ-H2AX (green). FIG. 25C is a graph quantitating about 90 cells (from three biological replicates) for γ-H2AX foci per cell between control (siCtrl) C9 KD (siC9) and DOX-treated U87 cells. Statistical significance was assessed by an unpaired t-test: NS P>0.05, *P≤0.05, **P≤0.01, ***P≤0.001 ****P≤0.0001.

### Example 8. C9 KD Leads to p53 Activation

One of the responses to DNA damage is activation of p53 by ATM. Upon DNA damage, ATM activation leads to p53 phosphorylation, which activates p53 as a transcription factor. Since increased DNA damage was observed upon C9 KD, whether p53 is activated in C9 depleted cells was investigated. First, total p53 levels were measured in U87 cells treated with control and two independent siRNAs against C9. WB analysis revealed that cells treated with the siRNAs targeting C9 elevates p53 levels. See FIG. 26. Then, the levels of phosphorylated (active) p53 (at s15) was measured. Phosphorylation of s15 on p53 was increased in both C9 siRNA samples. See FIG. 26. Increased s15 phosphorylation suggests that p53 is activated. WB analysis also revealed that C9 KD increased p21 protein levels in both siRNA treated U87 cells, consistent with p53 activation, since one of the targets of p53 is p21 (CDKN1A). FIG. 26 is a WB that shows the levels of phosphop-p53, p53, and p21 levels in control (siCtrl) and C9 KD cells treated with two independent siRNAs (siC9 #1 (SEQ ID NO:631) and siC9 #2 (SEQ ID NO:633)) with actin as loading control.

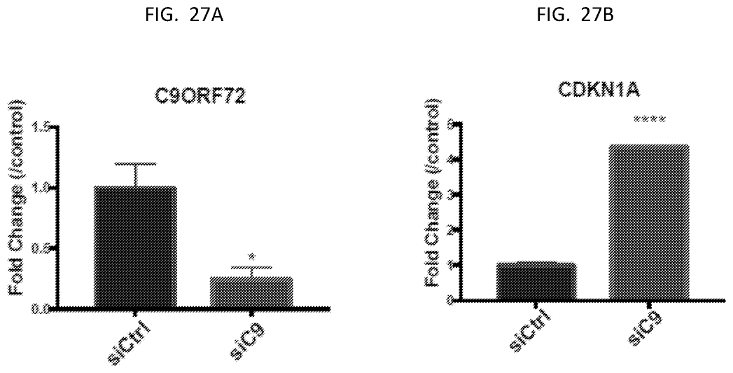

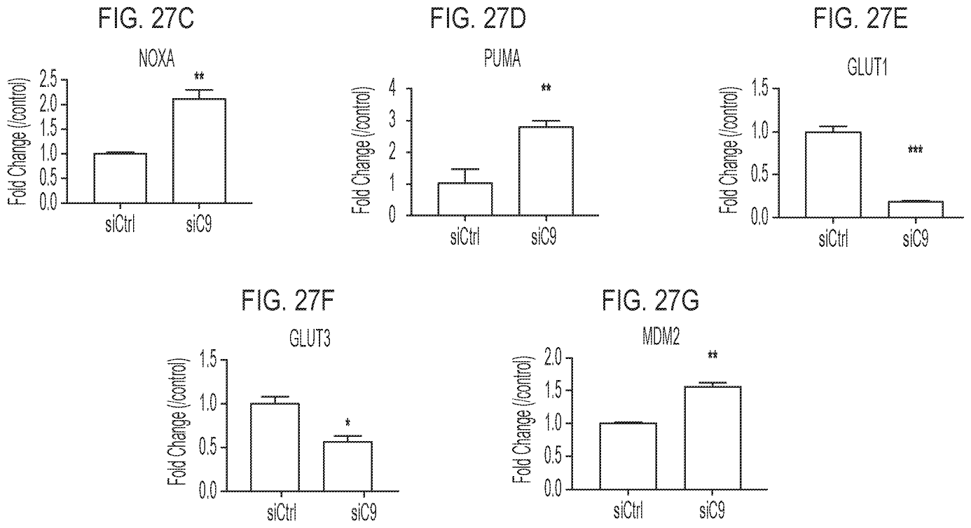

To further validate the activation of p53, the mRNA levels of several canonical p53 target genes, p21, MDM2, NOXA, PUMA, GLUT1 and GLUT3 were measured. qRT-PCR analysis revealed that the p53 target genes were elevated and the repression targets (GLUT1 and GLUT3) exhibited lower mRNA levels. See FIG. 27. This also is consistent with p53 activation. FIG. 27 presents RT-qPCR analysis of C9ORF72, CDKN1A (p21), NOXA, PUMA, GLUT1, MDM2 and GLUT3 mRNA levels in U87 cells treated with control (siCtrl) and C9 (siC9; SEQ ID NO:631) siRNA. These studies were performed using three biological replicates (n=3); error bars represent S.E.M. Statistical significance was assessed by an unpaired t-test: NS P>0.05, *P≤0.05, **P≤0.01, ***P≤0.001 ****P≤0.0001.

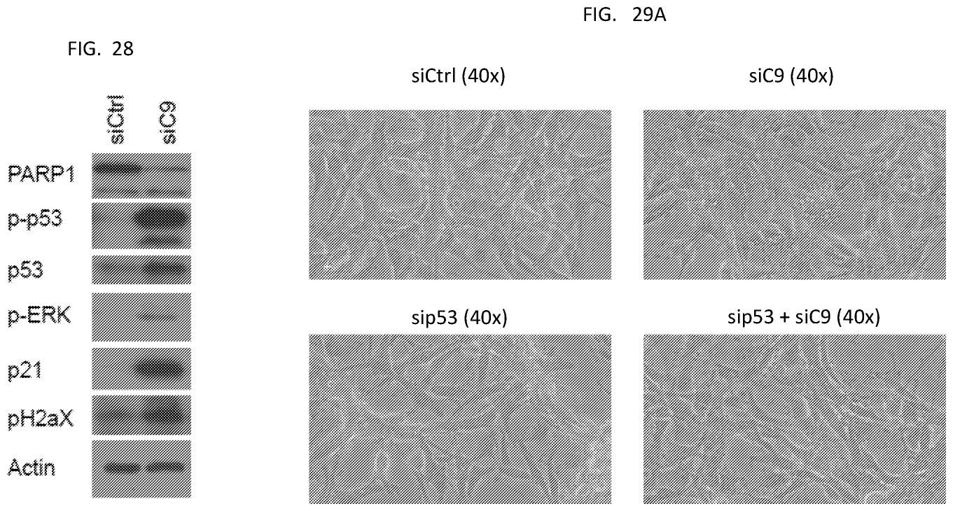

Whether p53 activation occurs in U2OS cells treated with C9 siRNA then was determined, since these cells exhibited a similar vacuolization phenotype. This WB analysis revealed that C9 KD in U2OS cells led to increased p53 levels and increases in phosphorylated p53 (on s15), also suggesting p53 activation. See FIG. 28. Additionally, an increase in CDKN1A/p21 protein levels was observed, which further supports p53 activation in U2OS cells. See FIG. 28. Furthermore, U2OS cells treated with C9 siRNAs showed PARP1 downregulation, and increases in p-ERK and in γ-H2AX, which also is observed in U87 cells upon C9 KD. See FIG. 28, a WB of U2OS cells treated with control and C9 siRNA and probed for PARP1, p-p53 (s15), p53, p-ERK, p21, p-H2AX, using actin as a loading control.

### Example 9. Cytoplasmic Vacuolization is p53 Dependent

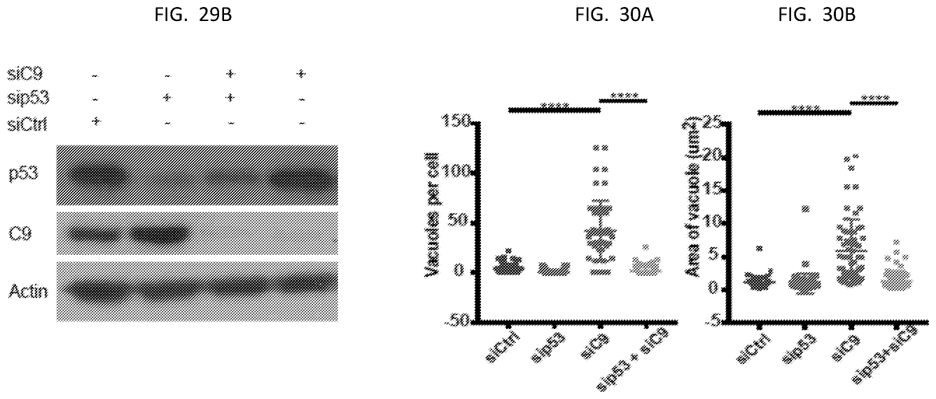

Whether methuosis-like cell death depends on p53 was next investigated. To test this, U87 cells were treated with control, C9, and p53 siRNAs. First, an increase in the amount of vacuoles per cell and in the size of the vacuoles upon C9 KD in U87 cells was reconfirmed. See FIG. 29 and FIG. 30. Cells treated only with p53 siRNA did not exhibit any significant changes in the amount of vacuoles or their size, however a co-KD of p53 and C9, was able to rescue the vacuolization phenotype. See FIG. 29 and FIG. 30. These results suggest that vacuole accumulation upon C9 KD depend on p53.

FIG. 29A shows phase contrast images (40×) of U87 cells treated with control (siCtrl), C9 (siC9; SEQ ID NO:631), p53 (sip53) or both C9 and p53 (siC9+sip53) siRNA as indicated. FIG. 29B presents a WB showing p53 and C9 levels under the conditions described above. FIG. 30A presents quantitation of data from about 50 cells from 3 biological replicates, indicating the number of vacuoles per cell for the conditions described for FIG. 29. FIG. 30B presents quantitation of data from about 50 cells from 3 biological replicates indicating the size of vacuoles in cells for conditions described in FIG. 29. Statistical significance was assessed by one way ANOVA: NS P>0.05, *P≤0.05, **P≤0.01, ***P≤0.001 ****P≤0.0001.

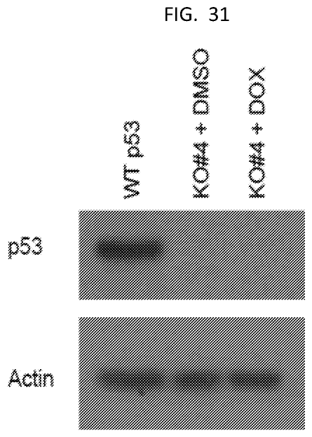

To further validate the dependency of vacuolization on p53, two p53 null cell lines were generated using CRISPR. First, the cells lack of p53 was validated by treating them doxorubicin, which induced p53. WB analysis revealed that doxorubicin treatment did not induce p53 accumulation, which shows these cell are p53 null. See FIG. 31, which is a WB showing p53 levels in wtp53 U87 (WT p53) cells and in p53 null U87 cells treated with DMSO (KO #4+DMSO) or DOX (KO #4+DOX).

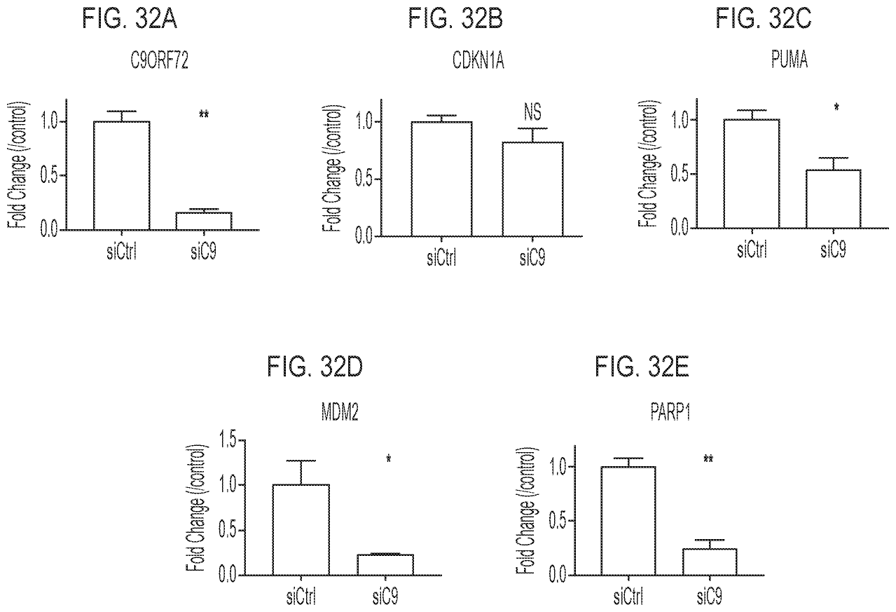

Additionally, C9 was knocked down in p53 null cells to validate that p53 targets are not induced. qRT-PCR analysis revealed that C9 KD in p53 null U87 cells did not result in a significant change in p21, but resulted in lower levels of PUMA and MDM2. See FIG. 32, which presents RT-qPCR analysis of C9ORF72, CDKN1A (p21), PUMA, MDM2 and PARP1 mRNA levels in U87 KO #4 cells treated with control (siCtrl) and C9 (siC9; SEQ ID NO:631) siRNA. These experiments were performed in three biological replicates (n=3) and error bars represent S.E.M. Statistical significance was assessed by unpaired t-test: NS P>0.05, *P≤0.05, **P≤0.01, ***P≤0.001 ****P≤0.0001.

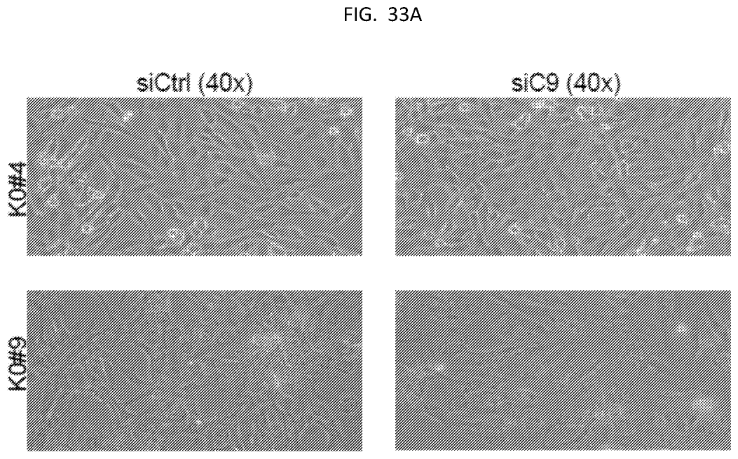

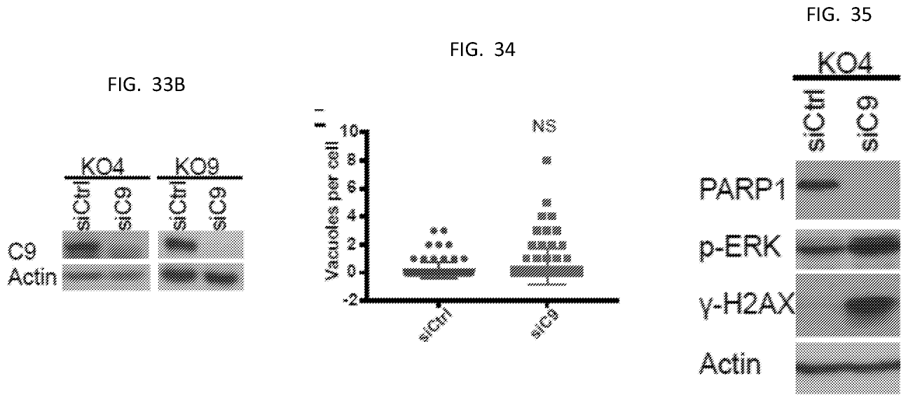

C9 then was knocked down with two independent siRNAs in the two U87 p53 null clones. In both p53 null U87 clones, no significant increase in vacuolization was observed, which further supports the role of p53 in vacuolization and macropinocytosis. See FIG. 33A, which provides phase contrast images (40×) of two clones (KO #4 and KO #9) of p53 null U87 cells generated by CRISPR, treated with control (siCtrl) and C9 (siC9; SEQ ID NO:631) siRNA, and FIG. 33B, a WB showing C9 levels after C9 KD in KO #4 cells.

FIG. 34 is a graph presenting quantitation of data for about 90 cells, indicating the number of vacuoles per cell. Statistical significance was assessed by unpaired t-test: NS P>0.05, *P≤0.05, **P≤0.01, ***P≤0.001 ****P≤0.0001. FIG. 35 is a WB showing PARP1, phospho-ERK and γ-H2AX levels upon C9 KD (siC9; SEQ ID NO:631) in U87 KO #4 cell line.

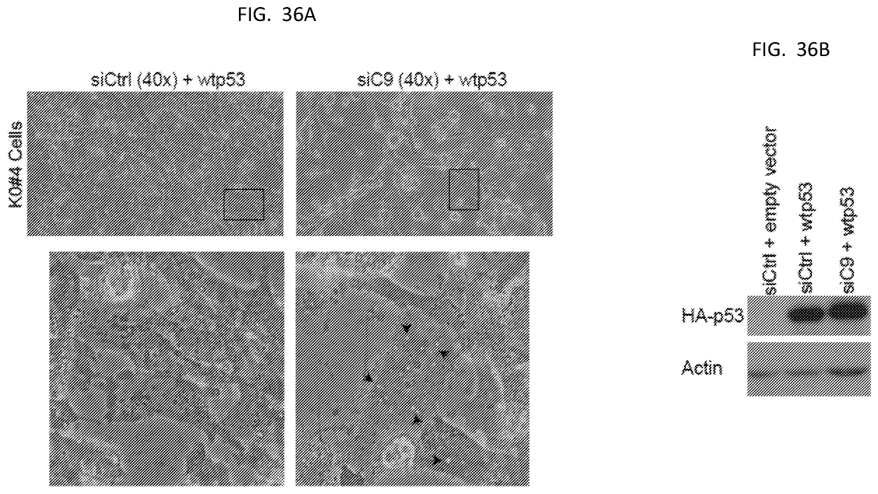

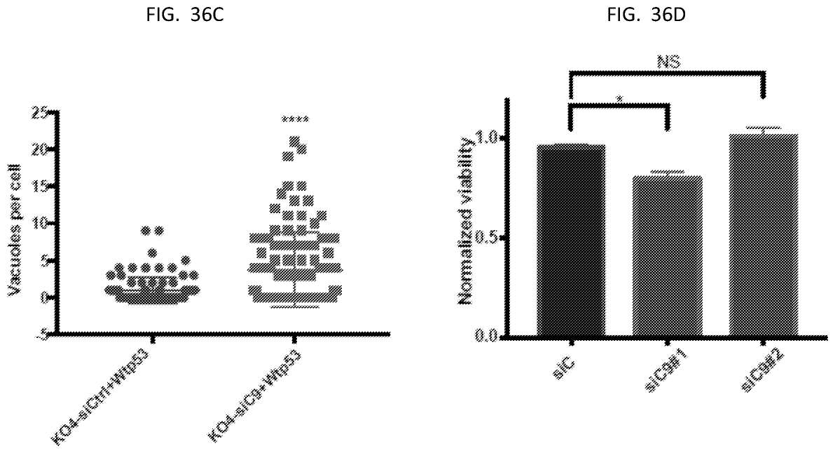

To provide further evidence of p53 involvement in vacuolization, it was tested whether p53 transfection into p53 null cell lines in C9 KD background induced accumulation of vacuoles. Quantitation of vacuoles in U87 p53 KO cell lines with transfected p53 revealed a significant increase upon C9 KD ‘1 (FIG. 29). In FIG. 36A, phase contrast images (40×) are shown of U87 p53 null KO #4 cells that were transfected with HA tagged wtp53 and then treated with control (siCtrl) or C9 (siC9; SEQ ID NO:631) siRNA. The black boxes show the location of the zoomed in images below with black arrows pointing to vacuoles. FIG. 36B is a WB demonstrating the success of p53 transfection by probing HA tagged p53. FIG. 36C is a graph that presents quantitation of data from about 100 cells, indicating the number of vacuoles per cell for conditions described above. Statistical significance was assessed by unpaired t-test: NS P>0.05, *P≤0.05, **P≤0.01, ***P≤0.001 ****P≤0.0001.

Even though a very significant increase in vacuole formation was seen, the reintroduction of p53 in C9 KD background did not fully recapitulate the vacuolization observed in wtp53 U87 cells treated with C9 siRNA. Though the accumulation of vacuoles is significant, the vacuoles in the transfected null cells were not as large as in the wtp53 cells and did not occupy the entire cell and therefore only partially rescued the vacuolization induction.

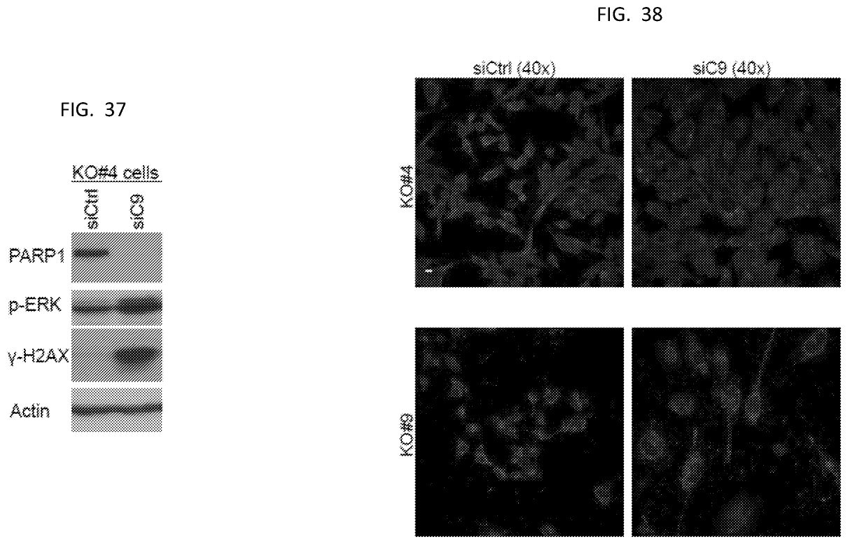

In addition, C9 was knocked down with two independent siRNAs in p53 null U87 cells to investigate the role of p53 in the viability of the cells. The viability assay revealed that KD of C9 in p53 null cells did not affect viability to the same extent as seen in wtp53 U87 cells (FIG. 36D). These results suggest that the both vacuolization and cell death are p53-dependent. Since the increase in vacuolization upon C9 KD seem to be p53-dependent, whether PARP1 downregulation, ERK and γ-H2AX phosphorylation is p53 dependent was also investigated. WB analysis on p53 KO U87 cells revealed that PARP downregulation occurred without the presence of p53, suggesting its p53 independent. See FIG. 37. PARP1 mRNA repression was also observed in the null cells (FIG. 32).

Additionally, increased ERK and γ-H2AX phosphorylation was observed, which suggests that these events are p53 independent. See FIG. 37, a WB of p53 null U87 cells (KO #4) treated with control and C9 siRNA and showing levels of PARP1, p-ERK, γ-H2AX. Actin was used as a loading control.

C9 then was knocked down in two p53 KO U87 cells lines which were imaged to show whether mitochondrial disorganization depends on p53. Image analysis revealed that C9 KD in p53 KO cells caused mitochondrial disorganization (FIG. 38), suggesting that mitochondrial defect upon C9 KD is p53-independent. FIG. 38 is a set of confocal microscopy images of two p53 null U87 clones (KO #4 and KO #9) treated with control and C9 siRNA, stained with Mitotracker red.

### Example 10. C9 KD Leads to EGF Repression

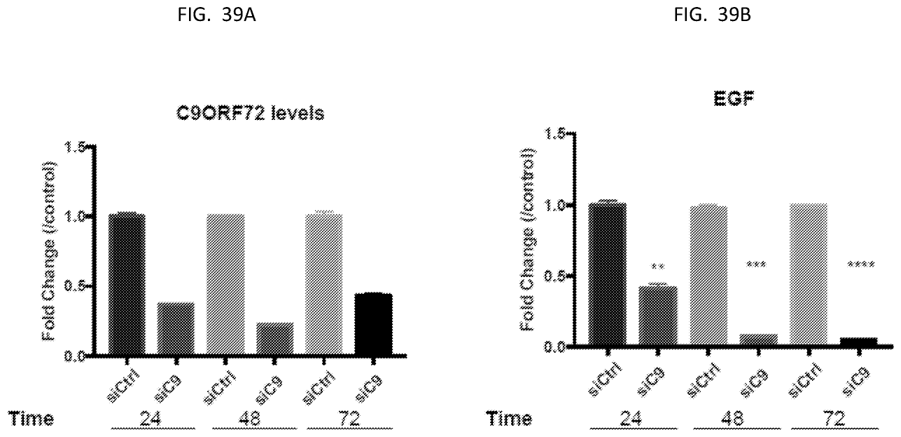

How C9 levels affect EGF signaling was investigated. C9 was knocked down, followed by determination of EGF mRNA levels every 24 hours. qRT-PCR analysis revealed that 24 hours after C9 KD, EGF mRNA levels were reduced by 50% (FIG. 39). Furthermore, after 48 and 72 hours, the reduction of EGF mRNA levels was about 97%. See FIG. 39, which presents data from qRT-PCR analysis, showing mRNA levels of C9ORF72 and EGF in U87 cells treated with control or C9 siRNA for 24, 48 and 72 hours (represented as fold change). Statistical significance was assessed by an unpaired t-test: NS P>0.05, *P≤0.05, **P≤0.01, ***P≤0.001 ****P 0.0001. These results suggest that C9 KD represses EGF mRNA and may act as an EGF signaling repressor and as an indirect EGFR inhibitor.

### Example 11. C9 KD Reduces Viability in BRCA1 Cells

Hepatocellular carcinoma (HCC1937, with BRCA1 mutation) cells were harvested using trypsin (0.5 ml per well) and the media was saved from each well. The saved media were resuspended with the trypsinised cells and 2-3 aliquots (0.05 ml each) of this suspension was transferred to 96-well plates. HCC cells were subjected to S1 and S2 as described above. A CellTiter-Glo™ Luminescent Viability assay was used to measure the viability of these aliquots by adding 0.15 mL of the CTG-media mixture to each well. The rest of the culture was used to extract protein. Viability of cells was measured using the 1:1 dilution of the CellTiter-Glo™ Luminescent reagent (Promega™) with media, which was read on a Victor 5™ plate reader after 10 minutes of shaking at room temperature. The intensity of luminescence was normalized to that of the DMSO control.

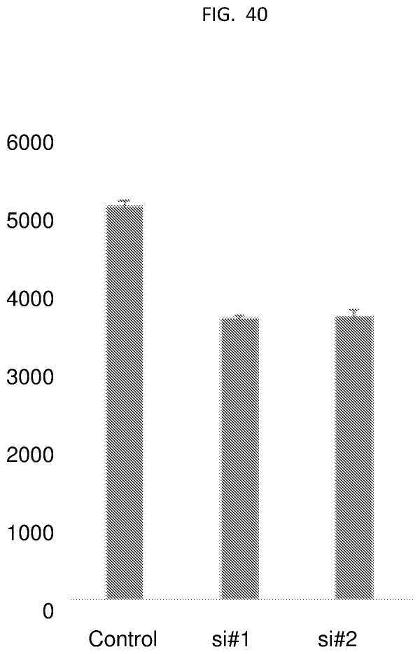

FIG. 40 shows that HCC1937 cells (with BRCA1 mutation) treated with siRNA (S1 and S2; SEQ ID NO:631) against C9 show a decrease in their viability (increased death). Without being bound to any particular theory, it is believed that this is because PARP1 is inhibited which is known to cause death in BRCA1 deficient breast cancer cell lines. These data show that C9 modulation can act as a PARP1 inhibitor.

### Example 12. Method of Treatment

In order to treat a PARP inhibition-susceptible cancer such as glioblastoma, breast cancers with BRCA mutations, osteosarcomas or melanomas, a patient in need is administered siRNA against C9. Optionally, additional agents are administered as well.

### Example 13. C9 siRNAs

See Table 3, below for sequences of C9 siRNAs contemplated for use with the invention.

TABLE 3
C9 siRNAs.

| Position in | | | SEQ ID |
|---|---|---|---|
| C9 | Type | Sequence | NO |
| | S 5′: | GGGCGAUCUUAACAUAAUA | 1 |
| | mRNA: | gggcgatcttaacataata | 2 |
| 1431-1453 | AS 3′: | CCCGCUAGAAUUGUAUUAU | 3 |
| | S 5′: | GGCGAUCUUAACAUAAUAA | 4 |
| | mRNA: | ggcgatcttaacataataa | 5 |
| 1432-1454 | AS 3′: | CCGCUAGAAUUGUAUUAUU | 6 |
| | S 5′: | GGCCUGCUAAAGGAUUCAA | 7 |
| | mRNA: | ggcctgctaaaggattcaa | 8 |
| 949-971 | AS 3′: | CCGGACGAUUUCCUAAGUU | 9 |
| | S 5′: | GUGCAGAGAAAGUAAAUAA | 10 |
| | mRNA: | gtgcagagaaagtaaataa | 11 |
| 824-846 | AS 3′: | CACGUCUCUUUCAUUUAUU | 12 |
| | S 5′: | CACCCUGUCAUGAACAUAU | 13 |
| | mRNA: | caccctgtcatgaacatat | 14 |
| 1061-1083 | AS 3′: | GUGGGACAGUACUUGUAUA | 15 |
| | S 5′: | GAGCCUUUAAAUGAUUUCA | 16 |
| | mRNA: | gagcctttaaatgatttca | 17 |
| 1995-2017 | AS 3′: | CUCGGAAAUUUACUAAAGU | 18 |
| | S 5′: | CCUGUCAUGAACAUAUUUA | 19 |
| | mRNA: | cctgtcatgaacatattta | 20 |
| 1064-1086 | AS 3′: | GGACAGUACUUGUAUAAAU | 21 |
| | S 5′: | GGGAGUGAUUAUUGUUUCA | 22 |
| | mRNA: | gggagtgattattgtttca | 23 |
| 375-397 | AS 3′: | CCCUCACUAAUAACAAAGU | 24 |
| 904-926 | S 5′: | GAAGCAGAAUCAUCAUUUA | 25 |
| | mRNA: | gaagcagaatcatcattta | 26 |
| | AS 3′: | CUUCGUCUUAGUAGUAAAU | 27 |
| | S 5′: | GCAACUCUGAGAUUAUAAA | 28 |
| | mRNA: | gcaactctgagattataaa | 29 |
| 2468-2490 | AS 3′: | CGUUGAGACUCUAAUAUUU | 30 |
| | S 5′: | GUGGUGCUAUAGAUGUAAA | 31 |
| | mRNA: | gtggtgctatagatgtaaa | 32 |
| 335-357 | AS 3′: | CACCACGAUAUCUACAUUU | 33 |
| | S 5′: | GCAGUGCAGAGAAAGUAAA | 34 |
| | mRNA: | gcagtgcagagaaagtaaa | 35 |
| 821-843 | AS 3′: | CGUCACGUCUCUUUCAUUU | 36 |
| | S 5′: | CUGCUUUCAUCUAUGAAAU | 37 |
| | mRNA: | ctgctttcatctatgaaat | 38 |
| 661-683 | AS 3′: | GACGAAAGUAGAUACUUUA | 39 |
| | S 5′: | GCUGUCAUGAAGGCUUUCU | 40 |
| | mRNA: | gctgtcatgaaggctttct | 41 |
| 746-768 | AS 3′: | CGACAGUACUUCCGAAAGA | 42 |
| | S 5′: | GGAGUGAUUAUUGUUUCAU | 43 |
| | mRNA: | ggagtgattattgtttcat | 44 |
| 376-398 | AS 3′: | CCUCACUAAUAACAAAGUA | 45 |
| | S 5′: | GUCUGUAGAAAUGUCUAAU | 46 |
| | mRNA: | gtctgtagaaatgtctaat | 47 |
| 2136-2158 | AS 3′: | CAGACAUCUUUACAGAUUA | 48 |
| | S 5: | CUCCGGAGCAUUUGGAUAA | 49 |
| | mRNA: | ctccggagcatttggataa | 50 |
| 66-88 | AS 3′: | GAGGCCUCGUAAACCUAUU | 51 |
| | S 5′: | GGGAUUCAGUCUGUAGAAA | 52 |
| | mRNA: | gggattcagtctgtagaaa | 53 |
| 2128-2150 | AS 3′: | CCCUAAGUCAGACAUCUUU | 54 |
| | S 5′: | GCUGAAACCUGGCUUAUCU | 55 |
| | mRNA: | gctgaaacctggcttatct | 56 |
| 1263-1285 | AS 3′: | CGACUUUGGACCGAAUAGA | 57 |
| | S 5′: | CAGUGUUCCUGAAGAAAUA | 58 |
| | mRNA: | cagtgttcctgaagaaata | 59 |
| 684-706 | AS 3′: | GUCACAAGGACUUCUUUAU | 60 |
| | S 5′: | GCCUAUUCCAUCACAAUCA | 61 |
| | mRNA: | gcctattccatcacaatca | 62 |
| 1568-1590 | AS 3′: | CGGAUAAGGUAGUGUUAGU | 63 |
| | S 5′: | CGGAGCAUUUGGAUAAUGU | 64 |
| | mRNA: | cggagcatttggataatgt | 65 |
| 69-91 | AS 3′: | GCCUCGUAAACCUAUUACA | 66 |
| | S 5′: | GGAGAAGUGAUUCCUGUAA | 67 |
| | mRNA: | ggagaagtgattcctgtaa | 68 |
| 637-659 | AS 3′: | CCUCUUCACUAAGGACAUU | 69 |
| 424-446 | S 5′: | CGCAGCACAUAUGGACUAU | 70 |
| | mRNA: | cgcagcacatatggactat | 71 |
| | AS 3′: | GCGUCGUGUAUACCUGAUA | 72 |
| | S 5′: | CUGGAUCAUACUCCAGAAU | 73 |
| | mRNA: | ctggatcatactccagaat | 74 |
| 1630-1652 | AS 3′: | GACCUAGUAUGAGGUCUUA | 75 |
| | S 5′: | CCUGAAGAUAGACCUUGAU | 76 |
| | mRNA: | cctgaagatagaccttgat | 77 |
| 1401-1423 | AS 3′: | GGACUUCUAUCUGGAACUA | 78 |
| | S 5′: | GCCUUAGAGAAUAUACUAA | 79 |
| | mRNA: | gccttagagaatatactaa | 80 |
| 2489-2511 | AS 3′: | CGGAAUCUCUUAUAUGAUU | 81 |
| | S 5′: | CUGUAGCUCAGUCAUUUAA | 82 |
| | mRNA: | ctgtagctcagtcatttaa | 83 |
| 3005-3027 | AS 3′: | GACAUCGAGUCAGUAAAUU | 84 |
| | S 5′: | CCACCACACACAUAGAUGU | 85 |
| | mRNA: | ccaccacacacatagatgt | 86 |
| 1016-1038 | AS 3′: | GGUGGUGUGUGUAUCUACA | 87 |
| | S 5′: | CCACCAAAUUUACACAACA | 88 |
| | mRNA: | ccaccaaatttacacaaca | 89 |
| 2878-2900 | AS 3′: | GGUGGUUUAAAUGUGUUGU | 90 |
| | S 5′: | GCUCAGGAUACGAUCAUCU | 91 |
| | mRNA: | gctcaggatacgatcatct | 92 |
| 1150-1172 | AS 3′: | CGAGUCCUAUGCUAGUAGA | 93 |
| | S 5′: | CACCAAAUUUACACAACAA | 94 |
| | mRNA: | caccaaatttacacaacaa | 95 |
| 2879-2901 | AS 3′: | GUGGUUUAAAUGUGUUGUU | 96 |
| | S 5′: | GCAGAUGUUUAAUUGGAAU | 97 |
| | mRNA: | gcagatgtttaattggaat | 98 |
| 2074-2096 | AS 3′: | CGUCUACAAAUUAACCUUA | 99 |
| | S 5′: | GUCCUAGAGUAAGGCACAU | 100 |
| | mRNA: | gtcctagagtaaggcacat | 101 |
| 218-240 | AS 3′: | CAGGAUCUCAUUCCGUGUA | 102 |
| | S 5′: | GACAGAGAUUGCUUUAAGU | 103 |
| | mRNA: | gacagagattgctttaagt | 104 |
| 147-169 | AS 3′: | CUGUCUCUAACGAAAUUCA | 105 |
| | S 5′: | GGCUGGUUUAUUGUACUGU | 106 |
| | mRNA: | ggctggtttattgtactgt | 107 |
| 3112-3134 | AS 3′: | CCGACCAAAUAACAUGACA | 108 |
| | S 5′: | GAGAGCCACUUCAGAAGAA | 109 |
| | mRNA: | gagagccacttcagaagaa | 110 |
| 1125-1147 | AS 3′: | CUCUCGGUGAAGUCUUCUU | 111 |
| | S 5′: | CCUGGCUUAUCUCUCAGAA | 112 |
| | mRNA: | cctggcttatctctcagaa | 113 |
| 1270-1292 | AS 3′: | GGACCGAAUAGAGAGUCUU | 114 |
| 638-660 | S 5′: | GAGAAGUGAUUCCUGUAAU | 115 |
| | mRNA: | gagaagtgattcctgtaat | 116 |
| | AS 3′: | CUCUUCACUAAGGACAUUA | 117 |
| | S 5′: | GUACCUGCUUUGGCAAUCA | 118 |
| | mRNA: | gtacctgctttggcaatca | 119 |
| 2447-2469 | AS 3′: | CAUGGACGAAACCGUUAGU | 120 |
| | S 5′: | CCUGGAUCAUACUCCAGAA | 121 |
| | mRNA: | cctggatcatactccagaa | 122 |
| 1629-1651 | AS 3′: | GGACCUAGUAUGAGGUCUU | 123 |
| | S 5′: | GGGUCAGAGUAUUAUUCCA | 124 |
| | mRNA: | gggtcagagtattattcca | 125 |
| 609-631 | AS 3′: | CCCAGUCUCAUAAUAAGGU | 126 |
| | S 5′: | GGAACAAGUUCAGAUUUCA | 127 |
| | mRNA: | ggaacaagttcagatttca | 128 |
| 2381-2403 | AS 3′: | CCUUGUUCAAGUCUAAAGU | 129 |
| | S 5′: | CUGGUUUAUUGUACUGUUA | 130 |
| | mRNA: | ctggtttattgtactgtta | 131 |
| 3114-3136 | AS 3′: | GACCAAAUAACAUGACAAU | 132 |
| | S 5′: | GGAUGAGCUUUAGAAAGAA | 133 |
| | mRNA: | ggatgagctttagaaagaa | 134 |
| 2045-2067 | AS 3′: | CCUACUCGAAAUCUUUCUU | 135 |
| | S 5′: | CCAGAAGAUUAUCUUAGAA | 136 |
| | mRNA: | ccagaagattatcttagaa | 137 |
| 567-589 | AS 3′: | GGUCUUCUAAUAGAAUCUU | 138 |
| | S 5′: | GUCAGGUGCAUCAUUACAU | 139 |
| | mRNA: | gtcaggtgcatcattacat | 140 |
| 1714-1736 | AS 3′: | CAGUCCACGUAGUAAUGUA | 141 |
| | S 5′: | GAAUGUGAAAGGUCAUAAU | 142 |
| | mRNA: | gaatgtgaaaggtcataat | 143 |
| 2910-2932 | AS 3′: | CUUACACUUUCCAGUAUUA | 144 |
| | S 5′: | GAACAUAGGAUGAGCUUUA | 145 |
| | mRNA: | gaacataggatgagcttta | 146 |
| 2038-2060 | AS 3′: | CUUGUAUCCUACUCGAAAU | 147 |
| | S 5′: | GAUGUCAGGUGCAUCAUUA | 148 |
| | mRNA: | gatgtcaggtgcatcatta | 149 |
| 1711-1733 | AS 3′: | CUACAGUCCACGUAGUAAU | 150 |
| | S 5′: | CAGCACAUAUGGACUAUCA | 151 |
| | mRNA: | cagcacatatggactatca | 152 |
| 426-448 | AS 3′: | GUCGUGUAUACCUGAUAGU | 153 |
| | S 5′: | CCUACUUUGUGGAUUUAGU | 154 |
| | mRNA: | cctactttgtggatttagt | 155 |
| 2105-2127 | AS 3′: | GGAUGAAACACCUAAAUCA | 156 |
| | S 5′: | GCAUGUGUAAACAUUGUUA | 157 |
| | mRNA: | gcatgtgtaaacattgtta | 158 |
| 3171-3193 | AS 3′: | CGUACACAUUUGUAACAAU | 159 |
| 495-517 | S 5′: | GUGUGUUGAUAGAUUAACA | 160 |
| | mRNA: | gtgtgttgatagattaaca | 161 |
| | AS 3′: | CACACAACUAUCUAAUUGU | 162 |
| | S 5′: | CAGAGGGCGAUCUUAACAU | 163 |
| | mRNA: | cagagggcgatcttaacat | 164 |
| 1427-1449 | AS 3′: | GUCUCCCGCUAGAAUUGUA | 165 |
| | S 5′: | GUGUGUAGAAUUACUGUAA | 166 |
| | mRNA: | gtgtgtagaattactgtaa | 167 |
| 1951-1973 | AS 3′: | CACACAUCUUAAUGACAUU | 168 |
| | S 5′: | CUGGGACAAUAUUCUUGGU | 169 |
| | mRNA: | ctgggacaatattcttggt | 170 |
| 201-223 | AS 3′: | GACCCUGUUAUAAGAACCA | 171 |
| | S 5′: | CCCACUUCAUAGAGUGUGU | 172 |
| | mRNA: | cccacttcatagagtgtgt | 173 |
| 480-502 | AS 3′: | GGGUGAAGUAUCUCACACA | 174 |
| | S 5′: | CUGGGAUUCAGUCUGUAGA | 175 |
| | mRNA: | ctgggattcagtctgtaga | 176 |
| 2126-2148 | AS 3′: | GACCCUAAGUCAGACAUCu | 177 |
| | S 5′: | GGUGUGUAGAAUUACUGUA | 178 |
| | mRNA: | ggtgtgtagaattactgta | 179 |
| 1950-1972 | AS 3′: | CCACACAUCUUAAUGACAU | 180 |
| | S 5′: | GAGUGGUGCUAUAGAUGUA | 181 |
| | mRNA: | gagtggtgctatagatgta | 182 |
| 333-355 | AS 3′: | CUCACCACGAUAUCUACAU | 183 |
| | S 5′: | GUACUGUUAUACAGAAUGu | 184 |
| | mRNA: | gtactgttatacagaatgt | 185 |
| 3124-3146 | AS 3′: | CAUGACAAUAUGUCUUACA | 186 |
| | S 5′: | CAGCGUAGAUACAUGAGAU | 187 |
| | mRNA: | cagcgtagatacatgagat | 188 |
| 1087-1109 | AS 3′: | GUCGCAUCUAUGUACUCUA | 189 |
| | S 5′: | GUCAGAGUAUUAUUCCAAU | 190 |
| | mRNA: | gtcagagtattattccaat | 191 |
| 611-633 | AS 3′: | CAGUCUCAUAAUAAGGUUA | 192 |
| | S 5′: | CACAGAGAGAAUGGAAGAU | 193 |
| | mRNA: | cacagagagaatggaagat | 194 |
| 588-610 | AS 3′: | GUGUCUCUCUUACCUUCUA | 195 |
| | S 5′: | CCAUCAUGAAUCAGAAAGA | 196 |
| | mRNA: | ccatcatgaatcagaaaga | 197 |
| 2936-2958 | AS 3′: | GGUAGUACUUAGUCUUUCU | 198 |
| | S 5′: | CACACUCUAAAUGGAGAAA | 199 |
| | mRNA: | cacactctaaatggagaaa | 200 |
| 298-320 | AS 3′: | GUGUGAGAUUUACCUCUUU | 201 |
| | S 5′: | GUAAGUUGUACAGUGAAAU | 202 |
| | mRNA: | gtaagttgtacagtgaaat | 203 |
| 3141-3163 | AS 3′: | CAUUCAACAUGUCACUUUA | 204 |
| 1521-1543 | S 5′: | GCAAGAACGAGAUGUUCUA | 205 |
| | mRNA: | gcaagaacgagatgttcta | 206 |
| | AS 3′: | CGUUCUUGCUCUACAAGAU | 207 |
| | S 5′: | CCACUUCAGAAGAAGACAU | 208 |
| | mRNA: | ccacttcagaagaagacat | 209 |
| 1130-1152 | AS 3′: | GGUGAAGUCUUCUUCUGUA | 210 |
| | S 5′: | CAGUGAUGGAGAAAUAACU | 211 |
| | mRNA: | cagtgatggagaaataact | 212 |
| 267-289 | AS 3′: | GUCACUACCUCUUUAUUGA | 213 |
| | S 5′: | CACUGACGAAAGCUUUACU | 214 |
| | mRNA: | cactgacgaaagctttact | 215 |
| 1170-1192 | AS 3′: | GUGACUGCUUUCGAAAUGA | 216 |
| | S 5′: | GACCUUUCUACACUAGUGU | 217 |
| | mRNA: | gacctttctacactagtgt | 218 |
| 1502-1524 | AS 3′: | CUGGAAAGAUGUGAUCACA | 219 |
| | S 5′: | CAGAGACACUCUAGUGAAA | 220 |
| | mRNA: | cagagacactctagtgaaa | 221 |
| 1221-1243 | AS 3′: | GUCUCUGUGAGAUCACUUU | 222 |
| | S 5′: | CUCUCAGCAAUUGCAGUUA | 223 |
| | mRNA: | ctctcagcaattgcagtta | 224 |
| 1653-1675 | AS 3′: | GAGAGUCGUUAACGUCAAU | 225 |
| | S 5′: | GCAUAAGGAAAGACAAGAA | 226 |
| | mRNA: | gcataaggaaagacaagaa | 227 |
| 543-565 | AS 3′: | CGUAUUCCUUUCUGUUCUU | 228 |
| | S 5′: | GUUGCUGUUUGCCUGCAAU | 229 |
| | mRNA: | gttgctgtttgcctgcaat | 230 |
| 2589-2611 | AS 3′: | CAACGACAAACGGACGUUA | 231 |
| | S 5′: | CAGGUUAUGUGAAGCAGAA | 232 |
| | mRNA: | caggttatgtgaagcagaa | 233 |
| 894-916 | AS 3′: | GUCCAAUACACUUCGUCUU | 234 |
| | S 5′: | GAUGAGCUUUAGAAAGAAA | 235 |
| | mRNA: | gatgagctttagaaagaaa | 236 |
| 2046-2068 | AS 3′: | CUACUCGAAAUCUUUCUUU | 237 |
| | S 5′: | CACCCACCAAAUUUACACA | 238 |
| | mRNA: | cacccaccaaatttacaca | 239 |
| 2875-2897 | AS 3′: | GUGGGUGGUUUAAAUGUGU | 240 |
| | S 5′: | GAAAGGGAUAAAGGUAAUA | 241 |
| | mRNA: | gaaagggataaaggtaata | 242 |
| 2840-2862 | AS 3′: | CUUUCCCUAUUUCCAUUAU | 243 |
| | S 5′: | CUUCCACAGACAGAACUUA | 244 |
| | mRNA: | cttccacagacagaactta | 245 |
| 451-473 | AS 3′: | GAAGGUGUCUGUCUUGAAU | 246 |
| | S 5′: | GUUGUACAGUGAAAUAAGU | 247 |
| | mRNA: | gttgtacagtgaaataagt | 248 |
| 3145-3167 | AS 3′: | CAACAUGUCACUUUAUUCA | 249 |
| 297-319 | S 5′: | CCACACUCUAAAUGGAGAA | 250 |
| | mRNA: | ccacactctaaatggagaa | 251 |
| | AS 3′: | GGUGUGAGAUUUACCUCUU | 252 |
| | S 5′: | CAGGAUACGAUCAUCUACA | 253 |
| | mRNA: | caggatacgatcatctaca | 254 |
| 1153-1175 | AS 3′: | GUCCUAUGCUAGUAGAUGU | 255 |
| | S 5′: | CUACCUCCCACUUCAUAGA | 256 |
| | mRNA: | ctacctcccacttcataga | 257 |
| 474-496 | AS 3′: | GAUGGAGGGUGAAGUAUCU | 258 |
| | S 5′: | GGUCAGAGUAUUAUUCCAA | 259 |
| | mRNA: | ggtcagagtattattccaa | 260 |
| 610-632 | AS 3′: | CCAGUCUCAUAAUAAGGUU | 261 |
| | S 5′: | GUGUGCAAGAACGAGAUGu | 262 |
| | mRNA: | gtgtgcaagaacgagatgt | 263 |
| 1517-1539 | AS 3′: | CACACGUUCUUGCUCUACA | 264 |
| | S 5′: | GACAACCACUGAACUAGAU | 265 |
| | mRNA: | gacaaccactgaactagat | 266 |
| 2976-2998 | AS 3′: | CUGUUGGUGACUUGAUCUA | 267 |
| | S 5′: | GUGGAUGUCAAUACUGUGA | 268 |
| | mRNA: | gtggatgtcaatactgtga | 269 |
| 1033-1055 | AS 3′: | CACCUACAGUUAUGACACU | 270 |
| | S 5′: | CAUCAUGAAUCAGAAAGAU | 271 |
| | mRNA: | catcatgaatcagaaagat | 272 |
| 2937-2959 | AS 3′: | GUAGUACUUAGUCUUUCUA | 273 |
| | S 5′: | CCUAUUCCAUCACAAUCAU | 274 |
| | mRNA: | cctattccatcacaatcat | 275 |
| 1569-1591 | AS 3′: | GGAUAAGGUAGUGUUAGUA | 276 |
| | S 5′: | CUACACUAGUGUGCAAGAA | 277 |
| | mRNA: | ctacactagtgtgcaagaa | 278 |
| 1509-1531 | AS 3′: | GAUGUGAUCACACGUUCUU | 279 |
| | S 5′: | GCUUUCAUCUAUGAAAUCA | 280 |
| | mRNA: | gctttcatctatgaaatca | 281 |
| 663-685 | AS 3′: | CGAAAGUAGAUACUUUAGU | 282 |
| | S 5′: | GCUUUCCCAUCAUGAAUCA | 283 |
| | mRNA: | gctttcccatcatgaatca | 284 |
| 2930-2952 | AS 3′: | CGAAAGGGUAGUACUUAGU | 285 |
| | S 5′: | CAUGAUUCAUGGUUUACAU | 286 |
| | mRNA: | catgattcatggtttacat | 287 |
| 1867-1889 | AS 3′: | GUACUAAGUACCAAAUGUA | 288 |
| | S 5′: | GGAAGACCUUUCUACACUA | 289 |
| | mRNA: | ggaagacctttctacacta | 290 |
| 1498-1520 | AS 3′: | CCUUCUGGAAAGAUGUGAU | 291 |
| | S 5′: | CUCUUCGGAACCUGAAGAU | 292 |
| | mRNA: | ctcttcggaacctgaagat | 293 |
| 1391-1413 | AS 3′: | GAGAAGCCUUGGACUUCUA | 294 |
| 1022-1044 | S 5′: | CACACAUAGAUGUGGAUGU | 295 |
| | mRNA: | cacacatagatgtggatgt | 296 |
| | AS 3′: | GUGUGUAUCUACACCUACA | 297 |
| | S 5′: | CUCCAGGUUAUGUGAAGCA | 298 |
| | mRNA: | ctccaggttatgtgaagca | 299 |
| 891-913 | AS 3′: | GAGGUCCAAUACACUUCGU | 300 |
| | S 5′: | GUUCUUGCUAUUGUUGAUA | 301 |
| | mRNA: | gttcttgctattgttgata | 302 |
| 2214-2236 | AS 3′: | CAAGAACGAUAACAACUAU | 303 |
| | S 5′: | GAACUGCUUUCAUCUAUGA | 304 |
| | mRNA: | gaactgctttcatctatga | 305 |
| 658-680 | AS 3′: | CUUGACGAAAGUAGAUACU | 306 |
| | S 5′: | GGAAGAAUAUGGAUGCAUA | 307 |
| | mRNA: | ggaagaatatggatgcata | 308 |
| 529-551 | AS 3′: | CCUUCUUAUACCUACGUAU | 309 |
| | S 5′: | GGACAAUAUUCUUGGUCCU | 310 |
| | mRNA: | ggacaatattcttggtcct | 311 |
| 204-226 | AS 3′: | CCUGUUAUAAGAACCAGGA | 312 |
| | S 5′: | GUGUUCCUGAAGAAAUAGA | 313 |
| | mRNA: | gtgttcctgaagaaataga | 314 |
| 686-708 | AS 3′: | CACAAGGACUUCUUUAUCU | 315 |
| | S 5′: | CGAAUGUGAAAGGUCAUAA | 316 |
| | mRNA: | cgaatgtgaaaggtcataa | 317 |
| 2909-2931 | AS 3′: | GCUUACACUUUCCAGUAUU | 318 |
| | S 5′: | GUGAGCUUGAACAUAGGAU | 319 |
| | mRNA: | gtgagcttgaacataggat | 320 |
| 2030-2052 | AS 3′: | CACUCGAACUUGUAUCCUA | 321 |
| | S 5′: | CUGAUACAGUACUCAAUGA | 322 |
| | mRNA: | ctgatacagtactcaatga | 323 |
| 710-732 | AS 3′: | GACUAUGUCAUGAGUUACU | 324 |
| | S 5′: | GCAAUAGGCUAUAAGGAAU | 325 |
| | mRNA: | gcaataggctataaggaat | 326 |
| 2603-2625 | AS 3′: | CGUUAUCCGAUAUUCCUUA | 327 |
| | S 5′: | CUUCUGCAAUCAACUGAAA | 328 |
| | mRNA: | cttctgcaatcaactgaaa | 329 |
| 1972-1994 | AS 3′: | GAAGACGUUAGUUGACUUU | 330 |
| | S 5′: | CAUGAUCGCUGGUAAAGUA | 331 |
| | mRNA: | catgatcgctggtaaagta | 332 |
| 1585-1607 | AS 3′: | GUACUAGCGACCAUUUCAU | 333 |
| | S 5′: | GAUGUGGACAGCUUGAUGU | 334 |
| | mRNA: | gatgtggacagcttgatgt | 335 |
| 2953-2975 | AS 3′: | CUACACCUGUCGAACUACA | 336 |
| | S 5′: | CACACAGUGUUCCUGAAGA | 337 |
| | mRNA: | cacacagtgttcctgaaga | 338 |
| 680-702 | AS 3′: | GUGUGUCACAAGGACUUCU | 339 |
| 1829-1851 | S 5′: | GAUGUAAACUUGACCACAA | 340 |
| | mRNA: | gatgtaaacttgaccacaa | 341 |
| | AS 3′: | CUACAUUUGAACUGGUGUU | 342 |
| | S 5′: | CUCCAGAAUUCUGCUCUCA | 343 |
| | mRNA: | ctccagaattctgctctca | 344 |
| 1640-1662 | AS 3′: | GAGGUCUUAAGACGAGAGU | 345 |
| | S 5′: | GAUAGACCUUGAUUUAACA | 346 |
| | mRNA: | gatagaccttgatttaaca | 347 |
| 1407-1429 | AS 3′: | CUAUCUGGAACUAAAUUGU | 348 |
| | S 5′: | CAGAGAGAAUGGAAGAUCA | 349 |
| | mRNA: | cagagagaatggaagatca | 350 |
| 590-612 | AS 3′: | GUCUCUCUUACCUUCUAGU | 351 |
| | S 5′: | CUUACUGGGACAAUAUUCU | 352 |
| | mRNA: | cttactgggacaatattct | 353 |
| 197-219 | AS 3′: | GAAUGACCCUGUUAUAAGA | 354 |
| | S 5′: | CAACCACUGAACUAGAUGA | 355 |
| | mRNA: | caaccactgaactagatga | 356 |
| 2978-3000 | AS 3′: | GUUGGUGACUUGAUCUACU | 357 |
| | S 5′: | GUGUCAAGGUGAAAUCUGA | 358 |
| | mRNA: | gtgtcaaggtgaaatctga | 359 |
| 1886-1908 | AS 3′: | CACAGUUCCACUUUAGACU | 360 |
| | S 5′: | GAAAGAAAGUGAGCUUGAA | 361 |
| | mRNA: | gaaagaaagtgagcttgaa | 362 |
| 2022-2044 | AS 3′: | CUUUCUUUCACUCGAACUU | 363 |
| | S 5′: | CUGGAGAAGUGAUUCCUGU | 364 |
| | mRNA: | ctggagaagtgattcctgt | 365 |
| 635-657 | AS 3′: | GACCUCUUCACUAAGGACA | 366 |
| | S 5′: | GACAGUUGGAAUGCAGUGA | 367 |
| | mRNA: | gacagttggaatgcagtga | 368 |
| 88-110 | AS 3′: | CUGUCAACCUUACGUCACU | 369 |
| | S 5′: | GGAUGCAUAAGGAAAGACA | 370 |
| | mRNA: | ggatgcataaggaaagaca | 371 |
| 539-561 | AS 3′: | CCUACGUAUUCCUUUCUGU | 372 |
| | S 5′: | CAUUGCAACUCUGAGAUUA | 373 |
| | mRNA: | cattgcaactctgagatta | 374 |
| 2464-2486 | AS 3′: | GUAACGUUGAGACUCUAAU | 375 |
| | S 5′: | GAUCGCUGGUAAAGUAGCU | 376 |
| | mRNA: | gatcgctggtaaagtagct | 377 |
| 1588-1610 | AS 3′: | CUAGCGACCAUUUCAUCGA | 378 |
| | S 5′: | CACACACAUAGAUGUGGAU | 379 |
| | mRNA: | cacacacatagatgtggat | 380 |
| 1020-1042 | AS 3′: | GUGUGUGUAUCUACACCUA | 381 |
| | S 5′: | CCUUCGAAAUGCAGAGAGU | 382 |
| | mRNA: | ccttcgaaatgcagagagt | 383 |
| 318-340 | AS 3′: | GGAAGCUUUACGUCUCUCA | 384 |
| 1681-1703 | S 5′: | CACUACAGUUCUCACAAGA | 385 |
| | mRNA: | cactacagttctcacaaga | 386 |
| | AS 3′: | GUGAUGUCAAGAGUGUUCU | 387 |
| | S 5′: | CACUGAACUAGAUGACUGU | 388 |
| | mRNA: | cactgaactagatgactgt | 389 |
| 2982-3004 | AS 3′: | GUGACUUGAUCUACUGACA | 390 |
| | S 5′: | GGUGAAAUCUGAGUUGGCU | 391 |
| | mRNA: | ggtgaaatctgagttggct | 392 |
| 1893-1915 | AS 3′: | CCACUUUAGACUCAACCGA | 393 |
| | S 5′: | CGGAAAGGAAGAAUAUGGA | 394 |
| | mRNA: | cggaaaggaagaatatgga | 395 |
| 523-545 | AS 3′: | GCCUUUCCUUCUUAUACCU | 396 |
| | S 5′: | GUGCAUCAUUACAUUGGGU | 397 |
| | mRNA: | gtgcatcattacattgggt | 398 |
| 1719-1741 | AS 3′: | CACGUAGUAAUGUAACCCA | 399 |
| | S 5′: | CUGUUGCCAAGACAGAGAU | 400 |
| | mRNA: | ctgttgccaagacagagat | 401 |
| 137-159 | AS 3′: | GACAACGGUUCUGUCUCUA | 402 |
| | S 5′: | CAUUGUAGUUACACAAACA | 403 |
| | mRNA: | cattgtagttacacaaaca | 404 |
| 2762-2784 | AS 3′: | GUAACAUCAAUGUGUUUGU | 405 |
| | S 5′: | GGAACCUGAAGAUAGACCU | 406 |
| | mRNA: | ggaacctgaagatagacct | 407 |
| 1397-1419 | AS 3′: | CCUUGGACUUCUAUCUGGA | 408 |
| | S 5′: | GUCAAGGUGAAAUCUGAGU | 409 |
| | mRNA: | gtcaaggtgaaatctgagt | 410 |
| 1888-1910 | AS 3′: | CAGUUCCACUUUAGACUCA | 411 |
| | S 5′: | CAUAGGAUGAGCUUUAGAA | 412 |
| | mRNA: | cataggatgagctttagaa | 413 |
| 2041-2063 | AS 3′: | GUAUCCUACUCGAAAUCUU | 414 |
| | S 5′: | CAGUACUCAAUGAUGAUGA | 415 |
| | mRNA: | cagtactcaatgatgatga | 416 |
| 716-738 | AS 3′: | GUCAUGAGUUACUACUACU | 417 |
| | S 5′: | CAAUAGGCUAUAAGGAAUA | 418 |
| | mRNA: | caataggctataaggaata | 419 |
| 2604-2626 | AS 3′: | GUUAUCCGAUAUUCCUUAU | 420 |
| | S 5′: | CCACACACAUAGAUGUGGA | 421 |
| | mRNA: | ccacacacatagatgtgga | 422 |
| 1019-1041 | AS 3′: | GGUGUGUGUAUCUACACCU | 423 |
| | S 5′: | CAACCACACUCUAAAUGGA | 424 |
| | mRNA: | caaccacactctaaatgga | 425 |
| 294-316 | AS 3′: | GUUGGUGUGAGAUUUACCu | 426 |
| | S 5′: | GAUUGCUUUAAGUGGCAAA | 427 |
| | mRNA: | gattgctttaagtggcaaa | 428 |
| 153-175 | AS 3′: | CUAACGAAAUUCACCGUUU | 429 |
| 727-749 | S 5′: | GAUGAUGAUAUUGGUGACA | 430 |
| | mRNA: | gatgatgatattggtgaca | 431 |
| | AS 3′: | CUACUACUAUAACCACUGU | 432 |
| | S 5′: | CAUAGAGUGUGUGUUGAUA | 433 |
| | mRNA: | catagagtgtgtgttgata | 434 |
| 487-509 | AS 3′: | GUAUCUCACACACAACUAU | 435 |
| | S 5′: | CUCUAGUGAAAGCCUUCCU | 436 |
| | mRNA: | ctctagtgaaagccttcct | 437 |
| 1229-1251 | AS 3′: | GAGAUCACUUUCGGAAGGA | 438 |
| | S 5′: | CACAGAGACACUCUAGUGA | 439 |
| | mRNA: | cacagagacactctagtga | 440 |
| 1219-1241 | AS 3′: | GUGUCUCUGUGAGAUCACU | 441 |
| | S 5′: | GUUAUGUGAAGCAGAAUCA | 442 |
| | mRNA: | gttatgtgaagcagaatca | 443 |
| 897-919 | AS 3′: | CAAUACACUUCGUCUUAGU | 444 |
| | S 5′: | CUACCUUGUAGUGUCCCAU | 445 |
| | mRNA: | ctaccttgtagtgtcccat | 446 |
| 3038-3060 | AS 3′: | GAUGGAACAUCACAGGGUA | 447 |
| | S 5′: | GAUUUCAAUUCCACAGAAA | 448 |
| | mRNA: | gatttcaattccacagaaa | 449 |
| 2007-2029 | AS 3′: | CUAAAGUUAAGGUGUCUUU | 450 |
| | S 5′: | GUUUCUACCUCCCACUUCA | 451 |
| | mRNA: | gtttctacctcccacttca | 452 |
| 470-492 | AS 3′: | CAAAGAUGGAGGGUGAAGU | 453 |
| | S 5′: | GGUCAUAAUAGCUUUCCCA | 454 |
| | mRNA: | ggtcataatagctttccca | 455 |
| 2920-2942 | AS 3′: | CCAGUAUUAUCGAAAGGGU | 456 |
| | S 5′: | CUUUCCGGCAAGUCAUGUA | 457 |
| | mRNA: | ctttccggcaagtcatgta | 458 |
| 986-1008 | AS 3′: | GAAAGGCCGUUCAGUACAU | 459 |
| | S 5′: | CGAAAGCUUUACUCCUGAU | 460 |
| | mRNA: | cgaaagctttactcctgat | 461 |
| 1176-1198 | AS 3′: | GCUUUCGAAAUGAGGACUA | 462 |
| | S 5′: | CUUUGGCAGAGCUAAGUUA | 463 |
| | mRNA: | ctttggcagagctaagtta | 464 |
| 2664-2686 | AS 3′: | GAAACCGUCUCGAUUCAAU | 465 |
| | S 5′: | GCUUUGGCAAUCAUUGCAA | 466 |
| | mRNA: | gctttggcaatcattgcaa | 467 |
| 2453-2475 | AS 3′: | CGAAACCGUUAGUAACGUU | 468 |
| | S 5′: | GCAUUUGGAUAAUGUGACA | 469 |
| | mRNA: | gcatttggataatgtgaca | 470 |
| 73-95 | AS 3′: | CGUAAACCUAUUACACUGU | 471 |
| | S 5′: | GUAAACUUGACCACAACUA | 472 |
| | mRNA: | gtaaacttgaccacaacta | 473 |
| 1832-1854 | AS 3′: | CAUUUGAACUGGUGUUGAU | 474 |
| 1182-1204 | S 5′: | CUUUACUCCUGAUUUGAAU | 475 |
| | mRNA: | ctttactcctgatttgaat | 476 |
| | AS 3′: | GAAAUGAGGACUAAACUUA | 477 |
| | S 5′: | CCCUUUAAAUCUCUUCGGA | 478 |
| | mRNA: | ccctttaaatctcttcgga | 479 |
| 1381-1403 | AS 3′: | GGGAAAUUUAGAGAAGCCU | 480 |
| | S 5′: | CAGUUAAGUAAGUUACACU | 481 |
| | mRNA: | cagttaagtaagttacact | 482 |
| 1666-1688 | AS 3′: | GUCAAUUCAUUCAAUGUGA | 483 |
| | S 5′: | CAUAAGGAAAGACAAGAAA | 484 |
| | mRNA: | cataaggaaagacaagaaa | 485 |
| 544-566 | AS 3′: | GUAUUCCUUUCUGUUCUUU | 486 |
| | S 5′: | CAAGUCAUGUAUGCUCCAU | 487 |
| | mRNA: | caagtcatgtatgctccat | 488 |
| 994-1016 | AS 3′: | GUUCAGUACAUACGAGGUA | 489 |
| | S 5′: | CACAGUUUCUACUUGUCCU | 490 |
| | mRNA: | cacagttactacttgtcct | 491 |
| 1301-1323 | AS 3′: | GUGUCAAAGAUGAACAGGA | 492 |
| | S 5′: | CAUCAGCUCACACUUGCAA | 493 |
| | mRNA: | catcagctcacacttgcaa | 494 |
| 774-796 | AS 3′: | GUAGUCGAGUGUGAACGUU | 495 |
| | S 5′: | CACAGAAAGAAAGUGAGCU | 496 |
| | mRNA: | cacagaaagaaagtgagct | 497 |
| 2018-2040 | AS 3′: | GUGUCUUUCUUUCACUCGA | 498 |
| | S 5′: | GUCUUACCAUGUACCUGCU | 499 |
| | mRNA: | gtcttaccatgtacctgct | 500 |
| 2437-2459 | AS 3′: | CAGAAUGGUACAUGGACGA | 501 |
| | S 5′: | CAAUUGCAGUUAAGUAAGU | 502 |
| | mRNA: | caattgcagttaagtaagt | 503 |
| 1660-1682 | AS 3′: | GUUAACGUCAAUUCAUUCA | 504 |
| | S 5′: | CUGUUCCGUUGUAGUAGGU | 505 |
| | mRNA: | ctgttccgttgtagtaggt | 506 |
| 801-823 | AS 3′: | GACAAGGCAACAUCAUCCA | 507 |
| | S 5′: | CUUUGAUGGAAACUGGAAU | 508 |
| | mRNA: | ctttgatggaaactggaat | 509 |
| 399-421 | AS 3′: | GAAACUACCUUUGACCUUA | 510 |
| | S 5′: | CAGAAGUACUUUCCUUGCA | 511 |
| | mRNA: | cagaagtactttccttgca | 512 |
| 1284-1306 | AS 3′: | GUCUUCAUGAAAGGAACGU | 513 |
| | S 5′: | CGAUCAUCUACACUGACGA | 514 |
| | mRNA: | cgatcatctacactgacga | 515 |
| 1160-1182 | AS 3′: | GCUAGUAGAUGUGACUGCU | 516 |
| | S 5′: | CAUGAAGGCUUUCUUCUCA | 517 |
| | mRNA: | catgaaggctttcttctca | 518 |
| 751-773 | AS 3′: | GUACUUCCGAAAGAAGAGU | 519 |
| 1645-1667 | S 5′: | GAAUUCUGCUCUCAGCAAU | 520 |
| | mRNA: | gaattctgctctcagcaat | 521 |
| | AS 3′: | CUUAAGACGAGAGUCGUUA | 522 |
| | S 5′: | CUUACACAGAGACACUCUA | 523 |
| | mRNA: | cttacacagagacactcta | 524 |
| 1215-1237 | AS 3′: | GAAUGUGUCUCUGUGAGAU | 525 |
| | S 5′: | CAAUCAUGAUCGCUGGUAA | 526 |
| | mRNA: | caatcatgatcgctggtaa | 527 |
| 1581-1603 | AS 3′: | GUUAGUACUAGCGACCAUU | 528 |
| | S 5′: | GCUAUAAGGAAUAGCAGGA | 529 |
| | mRNA: | gctataaggaatagcagga | 530 |
| 2610-2632 | AS 3′: | CGAUAUUCCUUAUCGUCCU | 531 |
| | S 5′: | CAAAGACAGAACAGGUACU | 532 |
| | mRNA: | caaagacagaacaggtact | 533 |
| 245-267 | AS 3′: | GUUUCUGUCUUGUCCAUGA | 534 |
| | S 5′: | GCUUUCUUCUCAAUGCCAU | 535 |
| | mRNA: | gctttcttctcaatgccat | 536 |
| 758-780 | AS 3′: | CGAAAGAAGAGUUACGGUA | 537 |
| | S 5′: | GCUAAAGGAUUCAACUGGA | 538 |
| | mRNA: | gctaaaggattcaactgga | 539 |
| 954-976 | AS 3′: | CGAUUUCCUAAGUUGACCU | 540 |
| | S 5′: | CUAGUGUGCAAGAACGAGA | 541 |
| | mRNA: | ctagtgtgcaagaacgaga | 542 |
| 1514-1536 | AS 3′: | GAUCACACGUUCUUGCUCU | 543 |
| | S 5′: | GAUCAGGUCUUUCAGCUGA | 544 |
| | mRNA: | gatcaggtctttcagctga | 545 |
| 1249-1271 | AS 3′: | CUAGUCCAGAAAGUCGACU | 546 |
| | S 5′: | CUAAUUACUUGGAACAAGU | 547 |
| | mRNA: | ctaattacttggaacaagt | 548 |
| 2371-2393 | AS 3′: | GAUUAAUGAACCUUGUUCA | 549 |
| | S 5′: | GUUCGAAUGUGAAAGGUCA | 550 |
| | mRNA: | gttcgaatgtgaaaggtca | 551 |
| 2906-2928 | AS 3′: | CAAGCUUACACUUUCCAGU | 552 |
| | S 5′: | GUGUAACUUAAUAAGCCUA | 553 |
| | mRNA: | gtgtaacttaataagccta | 554 |
| 1554-1576 | AS 3′: | CACAUUGAAUUAUUCGGAU | 555 |
| | S 5′: | GUAGAUACAUGAGAUCCGA | 556 |
| | mRNA: | gtagatacatgagatccga | 557 |
| 1091-1113 | AS 3′: | CAUCUAUGUACUCUAGGCU | 558 |
| | S 5′: | GAAGUACUUUCCUUGCACA | 559 |
| | mRNA: | gaagtactttccttgcaca | 560 |
| 1286-1308 | AS 3′: | CUUCAUGAAAGGAACGUGU | 561 |
| | S 5′: | CUAUGUAGUUGAGCUCUGU | 562 |
| | mRNA: | ctatgtagttgagctctgt | 563 |
| 2235-2257 | AS 3′: | GAUACAUCAACUCGAGACA | 564 |
| 807-829 | S 5′: | CGUUGUAGUAGGUAGCAGU | 565 |
| | mRNA: | cgttgtagtaggtagcagt | 566 |
| | AS 3′: | GCAACAUCAUCCAUCGUCA | 567 |
| | S 5′: | CCUUCUUGCAUUUCUGCCU | 568 |
| | mRNA: | ccttcttgcatttctgcct | 569 |
| 2556-2578 | AS 3′: | GGAAGAACGUAAAGACGGA | 570 |
| | S 5′: | GUUCAGAUUUCACUGGUCA | 571 |
| | mRNA: | gttcagatttcactggtca | 572 |
| 2388-2410 | AS 3′: | CAAGUCUAAAGUGACCAGU | 573 |
| | S 5′: | CAAUGAUGAUGAUAUUGGU | 574 |
| | mRNA: | caatgatgatgatattggt | 575 |
| 723-745 | AS 3′: | GUUACUACUACUAUAACCA | 576 |
| | S 5′: | CUUGAACAUAGGAUGAGCU | 577 |
| | mRNA: | cttgaacataggatgagct | 578 |
| 2035-2057 | AS 3′: | GAACUUGUAUCCUACUCGA | 579 |
| | S 5′: | GAGAAUGGAAGAUCAGGGU | 580 |
| | mRNA: | gagaatggaagatcagggt | 581 |
| 594-616 | AS 3′: | CUCUUACCUUCUAGUCCCA | 582 |
| | S 5′: | CAUAAUAGCUUUCCCAUCA | 583 |
| | mRNA: | cataatagctttcccatca | 584 |
| 2923-2945 | AS 3′: | GUAUUAUCGAAAGGGUAGU | 585 |
| | S 5′: | GAUAAUGUGACAGUUGGAA | 586 |
| | mRNA: | gataatgtgacagttggaa | 587 |
| 80-102 | AS 3′: | CUAUUACACUGUCAACCUU | 588 |
| | S 5′: | GAAUAUGGAUGCAUAAGGA | 589 |
| | mRNA: | gaatatggatgcataagga | 590 |
| 533-555 | AS 3′: | CUUAUACCUACGUAUUCCU | 591 |
| | S 5′: | CAAUAUUCUUGGUCCUAGA | 592 |
| | mRNA: | caatattcttggtcctaga | 593 |
| 207-229 | AS 3′: | GUUAUAAGAACCAGGAUCU | 594 |
| | S 5′: | GAGAUUGCUUUAAGUGGCA | 595 |
| | mRNA: | gagattgctttaagtggca | 596 |
| 151-173 | AS 3′: | CUCUAACGAAAUUCACCGU | 597 |
| | S 5′: | CAGAAGAAGACAUGGCUCA | 598 |
| | mRNA: | cagaagaagacatggctca | 599 |
| 1136-1158 | AS 3′: | GUCUUCUUCUGUACCGAGU | 600 |
| | S 5′: | CUUCUUGCAUUUCUGCCUA | 601 |
| | mRNA: | cttcttgcatttctgccta | 602 |
| 2557-2579 | AS 3′: | GAAGAACGUAAAGACGGAU | 603 |
| | S 5′: | CUUUGUACAAGGCCUGCUA | 604 |
| | mRNA: | ctttgtacaaggcctgcta | 605 |
| 939-961 | AS 3′: | GAAACAUGUUCCGGACGAU | 606 |
| | S 5′: | GAAUGGAAGAUCAGGGUCA | 607 |
| | mRNA: | gaatggaagatcagggtca | 608 |
| 596-618 | AS 3′: | CUUACCUUCUAGUCCCAGU | 609 |
| 2694-2716 | S 5′: | CUUAAUGCGUUUGGACCAU | 610 |
| | mRNA: | cttaatgcgtttggaccat | 611 |
| | AS 3′: | GAAUUACGCAAACCUGGUA | 612 |
| | S 5′: | CUAAAGGAUUCAACUGGAA | 613 |
| | mRNA: | ctaaaggattcaactggaa | 614 |
| 955-977 | AS 3′: | GAUUUCCUAAGUUGACCUU | 615 |
| | S 5′: | GAUUAUCUUAGAAGGCACA | 616 |
| | mRNA: | gattatcttagaaggcaca | 617 |
| 573-595 | AS 3′: | CUAAUAGAAUCUUCCGUGU | 618 |
| | S 5′: | CAUUUGGGCUCCAAAGACA | 619 |
| | mRNA: | catttgggctccaaagaca | 620 |
| 234-256 | AS 3′: | GUAAACCCGAGGUUUCUGU | 621 |
| | S 5′: | CAAAUCACCUUUAUUAGCA | 622 |
| | mRNA: | caaatcacctttattagca | 623 |
| 168-190 | AS 3′: | GUUUAGUGGAAAUAAUCGU | 624 |
| | S 5′: | CAAAUUGAAAUGUGCACCU | 625 |
| | mRNA: | caaattgaaatgtgcacct | 626 |
| 2325-2347 | AS 3′: | GUUUAACUUUACACGUGGA | 627 |
| | S 5′: | CUUUGUGGAUUUAGUCCCU | 628 |
| | mRNA: | ctttgtggatttagtccct | 629 |
| 2109-2131 | AS 3′: | GAAACACCUAAAUCAGGGA | 630 |

S indicates sense, AS indicates antisense.

As will be apparent to one of ordinary skill in the art from a reading of this disclosure, further embodiments of the present invention can be presented in forms other than those specifically disclosed above. The particular embodiments described above are, therefore, to be considered as illustrative and not restrictive. Those skilled in the art will recognize, or be able to ascertain, using no more than routine experimentation, numerous equivalents to the specific embodiments described herein. Such equivalents are considered to be within the scope of this invention. Numerous changes in the details of implementation of the invention can be made without departing from the spirit and scope of the invention, which is limited only by the claims that follow. Features of the disclosed embodiments can be combined and rearranged in various ways within the scope and spirit of the invention. The scope of the invention is as set forth in the appended claims and equivalents thereof, rather than being limited to the examples contained in the foregoing description.

### REFERENCES

All references listed below and throughout the specification are hereby incorporated by reference in their entirety.
Arcamone et al., Biotechnol. Bioeng. 11:1101-1110, 1969.
Bachmann et al., Proc. Natl. Acad. Sci. USA 110:8531-8536, 2013.
Bartel and Szostak, Science 261:1411-1418, 1993.
Baulcombe, 1996.
Bertrand et al., Exp. Cell Res. 211:314-321, 1994.
Caplan et al., 2001.
Cech et al., U.S. Pat. No. 4,987,071.
Cech et al., U.S. Pat. No. 5,116,742.
Cheng et al., J. Biol. Chem. 281:17718-17726, 2006.
Cheng et al., J. Clin. Invest. 101:1992-1999, 1998.
Chi et al., Oncogene, 18:2281-2290, 1999.
Cogoni et al., 1994.
d'Adda di Fagagna et al., Nature Gen., 23(1): 76-80, 1999.
DeJesus-Hernandez et al., Neuron 72 (2): 245-56, 2011.
Elbashir et al., 2001.
Elbashir et al., 2001.
Farg et al., Human Molecular Genetics 23:3579-3595, 2014.
Gaultier et al., Nucleic Acids. Res. 15:6625-6641, 1987.
Haselhoff and Gerlach, Nature 334:585-591, 1988.
Hewlett et al., J. Cell Biol. 124:689-703, 1994.
Higgins et al., BMC Neurosci 4:16, 2003.
Inoue et al., Nucleic Acids Res. 15:6131-6148, 1987.
Inoue et al., FEBS Lett. 215:327-330, 1987.
Jensen, Biochim. Biophys. Acta 122:167-174, 1966.
Kalyanaraman et al., Free Radic. Biol. Med. 52:1-6, 2012.
Katz et al., Genes Dev. 32:430-447, 2018.
Kennerdell, 1998.
Kimura et al., Autophagy 3:452-460, 2007.
Koivusalo et al., J. Cell Biol. 188:547-563, 2010.
Kruhn et al., Cell Cycle 8, 2009.
Levine et al., Bioinformatics 29:499-503, 2013.
Lim and Gleeson, Immunol. Cell Biol. 89:836-843, 2011.
Maltese and Overmeyer, Am J. Pathol. 184(6):1630-1642, 2014.
Murphy, Biochemical J. 417:1-13, 2009.
O'Rourke et al., Science 351:1324-1329, 2016.
Overmeyer et al., Cell Signal 19:1034-1043, 2007.
Overmeyer et al., Mol. Cancer Res. 6:965-977, 2008.
Overmeyer et al., Mol. Cancer 10, 69, 2011.
Rajasekharan et al. Sci. Rep. 7:6803, 2017.
Renton et al., Neuron 72 (2): 257-68), 2011.
Riancho et al., Frontiers in Cellular Neuroscience 9, 2015.
Rodier and Campisi, J. Cell. Biol. 192:547-556, 2011.
Sakellariou et al, Sci Rep 6:1309, 2016.
Sasaki et al., J. Neuropathol. Exp. Neurol. 66:10-16, 2007.
Sellier et al., EMBO J. 35:1251-1363, 2016.
Svoboda et al., 2000.
Tchkonia et al., J. Clin. Invest. 123:966-972, 2013.
Timmons, 1998.
Webster et al., EMBO J. 35:1483-1595, 2016.
Wong et al., Neuron 14:1105-1116, 1995.
Waterhouse et al., 1998.
West et al., J. Cell. Biol. 109:2731-2739, 1989.
Wianny and Zernicka-Goetz, 2000.
Xiang et al., Meth. Mol. Biol. 487:147-160, 2009.
Yang et al., 2001.
Yang et al., Biochim. Biophys. Acta 1845:84-89, 2014.
Yang et al., Sci. Adv. 2:e1601167-e160116, 2016.
United States Patent Publication No. 2004/0023390.

1. A method of treating a cancer susceptible to methuosis in a subject in need, comprising administering a therapeutically effective amount of an agent selected from the group consisting of an inhibitory oligonucleotide (IO) that reduces C9 expression, an anti-C9 antibody, and a combination thereof.

2. The method of claim 1, wherein the cancer is colon adenocarcinoma, esophagus adenocarcinoma, liver hepatocellular carcinoma, squamous cell carcinoma, pancreas adenocarcinoma, islet cell tumor, rectum adenocarcinoma, gastrointestinal stromal tumor, stomach adenocarcinoma, adrenal cortical carcinoma, follicular carcinoma, papillary carcinoma, breast cancer, ductal carcinoma, lobular carcinoma, melanoma, intraductal carcinoma, mucinous carcinoma, phyllodes tumor, ovarian cancer including ovarian adenocarcinoma, endometrium adenocarcinoma, granulose cell tumor, mucinous cystadenocarcinoma, cervix adenocarcinoma, vulva squamous cell carcinoma, basal cell carcinoma, prostate cancer, giant cell tumor of bone, bone osteosarcoma, larynx carcinoma, lung adenocarcinoma, kidney carcinoma, urinary bladder carcinoma, Wilm's tumor, uterine cancer, endometrial cancer and lymphoma.

3. The method of claim 1, wherein the cancer is selected from the group consisting of BRCA 1- or 2-associated ovarian or breast cancer, glioblastoma, and melanoma.

4. The method of claim 1, wherein the IO is an siRNA, an shRNA, an antisense molecule, an miRNA or a ribozyme.

5. The method of claim 4, wherein the IO is an siRNA.

6. The method of claim 4, wherein the IO is an siRNA comprising SEQ ID NO. 631 or a biologically active fragment or variant thereof.

7. The method of claim 5, wherein the siRNA is selected from the group consisting of SEQ ID NOs:1-634.

8. The method of claim 5, wherein the siRNA is selected from the group consisting of SEQ ID NOs:8, 22, 23, 24, 26, 76, 77, 78, 112, 131, 167, 334, 335, 336, 391, 392, 393, 499, 500, 501, 631, and 633.

9. The method of claim 1, wherein the anti-C9 antibody is a monoclonal antibody.

10. The method of claim 1, further comprising co-administering a therapeutically effective amount of an agent selected from the group consisting of a PARP inhibitor, an adjunct cancer therapeutic agent, and both.

11. The method of claim 10, wherein the adjunct cancer therapeutic agent comprises an antitumor alkylating agent, antitumor antimetabolite, antitumor antibiotics, plant-derived antitumor agent, antitumor platinum complex, antitumor campthotecin derivative, antitumor tyrosine kinase inhibitor, monoclonal or polyclonal antibody, interferon, biological response modifier, hormonal anti-tumor agent, anti-tumor viral agent, angiogenesis inhibitor, differentiating agent, PI3K/mTOR/AKT inhibitor, cell cycle inhibitor, apoptosis inhibitor, hsp 90 inhibitor, tubulin inhibitor, DNA repair inhibitor, anti-angiogenic agent, receptor tyrosine kinase inhibitor, topoisomerase inhibitor, taxane, agent targeting Her2, hormone antagonist, agent targeting a growth factor receptor, or a pharmaceutically acceptable salt thereof.

12. The method of claim 1 further comprising treating PARP inhibition-susceptible cancer with at least one adjunct cancer therapy protocol selected from the group consisting of surgery, radiation therapy, chemotherapy, gene therapy, DNA therapy, adjuvant therapy, neoadjuvant therapy, viral therapy, RNA therapy, immunotherapy, and nanotherapy.

13. An isolated siRNA molecule comprising the sequence of SEQ ID NO:631.

14. An isolated siRNA molecule selected from the group consisting of SEQ ID NOs:8, 22, 23, 24, 26, 76, 77, 78, 112, 131, 167, 334, 335, 336, 391, 392, 393, 499, 500, 501, 631, and 633.

15. A pharmaceutical composition comprising a pharmaceutically acceptable carrier and an siRNA that reduces C9 expression.

16. The pharmaceutical composition of claim 15, wherein the siRNA is SEQ ID NO:631.

17. The pharmaceutical composition of claim 15, which further comprises an agent selected from the group consisting of a PARP inhibitor, an anti-C9 antibody, and both.
